(19) United States
(12) Patent Application Publication
(10) Pub. No.: US 2018/0346408 A1
(43) Pub. Date: Dec. 6, 2018

(54) LIPID COMPOUNDS AND COMPOSITIONS AND THEIR OPTHALMIC USE

(71) Applicant: BIOZEP AS, Oslo (NO)

(72) Inventor: Anne Kristin Holmeide, Oslo (NO)

(21) Appl. No.: 15/779,829

(22) PCT Filed: Nov. 30, 2016
(86) PCT No.: PCT/GB2016/053769

§ 371 Date: May 29, 2018

(30) Foreign Application Priority Data
Nov. 30, 2015 (GB) 1521085.9

(51) Int. Cl. C07C 229/22; C07C 229/30; C07C 323/52; C07C 323/54; C07C 323/58; C07C 59/76; C07C 59/60; C07C 59/56; C07C 57/52; C07C 57/03; C07C 233/09; A61P 27/12; A61P 27/06

(52) U.S. Cl. CPC C07C 229/22; C07C 229/30; C07C 323/52; C07C 323/54; C07C 323/58; C07C 59/76; A61P 27/06; C07C 59/56; C07C 57/52; C07C 57/03; C07C 233/09; A61P 27/12; C07C 59/60

(57) ABSTRACT

The invention relates to lipid compounds of formula (I) and their pharmaceutically acceptable salts for the prevention and/or treatment of ophthalmic disorders such as retinal degenerative disorders and ocular inflammatory diseases: (I) (wherein R¹ is either a C₉ to C₂₂ alkyl group, or a C₉ to C₂₂ alkenyl group having from 1 to 6 double bonds; R² is selected from the group consisting of a halogen atom, a hydroxy group, an alkyl group, an alkoxy group, an alkylthio group, a carboxy group, an acyl group, an amino group, and an alkylamino group; R³ is a hydrogen atom, or a group R²; R⁴ is a carboxylic acid or a derivative thereof; and X is methylene (—CH₂—), or an oxygen or sulfur atom).

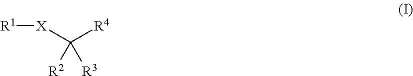

### FIELD OF INVENTION

The present invention relates to the use of fatty acid derivatives in the treatment and/or prevention of ophthalmic disorders, in particular retinal disorders such as age-related macular degeneration and diabetic retinopathy, and ocular inflammatory diseases. It further relates to novel omega-6 polyunsaturated fatty acid derivatives, to pharmaceutical compositions containing them, and to their use in such treatment.

### BACKGROUND OF THE INVENTION

Millions of people live with varying degrees of irreversible vision loss because they have an untreatable, degenerative eye disorder which affects the retina. In these conditions, the delicate layer of tissue that lines the inside back of the eye is damaged affecting its ability to send light signals to the brain. Diseases of the retina and retinal function can lead to permanent loss of visual function for which there is no definitive treatment. Vision loss has serious consequences for the individual as well as society. Reduced vision among mature adults has been shown to result in social isolation, family stress, and ultimately a greater tendency to experience other health conditions. Experts predict that by 2030 rates of vision loss will double along with the aging population.

According to Prevent Blindness America (2008), the four leading eye diseases affecting older Americans are age-related macular degeneration (AMD), cataracts, diabetic retinopathy, and glaucoma. As people age, they are far more likely to have serious age-related eye conditions.

AMD is a leading cause of blindness in adults over 55 years of age in the developed world. It afflicts an estimated 30 to 50 million people worldwide and is the leading cause of severe vision loss in Western societies. AMD causes the loss of photoreceptor cells in the central part of the retina (the macula) which is the part of the retina that supplies high acuity central vision. The loss of central vision affects activities such as reading, driving and face recognition, and has a significant negative impact on daily function and quality of life.

Macular degeneration can be classified into two types: dry-form and wet-form. The dry-form is more common than the wet: about 90% of age-related macular degeneration patients are diagnosed with the dry-form. The wet-form of the disease and geographic atrophy, which is the end-stage phenotype of dry-form AMD, causes the most serious vision loss. All patients who develop wet-form AMD are believed to previously have developed dry-form AMD for a prolonged period of time. The exact causes of AMD are still unknown. Although some treatments to slow progression are available for wet AMD, there is currently no cure for this irreversible disease. For the most advanced form, wet AMD, anti-VEGF therapy dominates the market. Despite this significant treatment advancement for wet AMD, sustained visual acuity improvements can only be maintained with burdensome monthly dosing and monitoring. For the vast majority of patients who have the dry form of AMD, no effective treatment is yet available. Because the dry-form of AMD precedes development of the wet-form, therapeutic intervention to prevent or delay disease progression in the dry-form of AMD would benefit those patients with the dry-form of AMD and could serve to reduce the incidence of the wet-form.

The pathological mechanism(s) for AMD have not been definitively elucidated. However, converging evidence from multiple studies implicates mitochondrial dysfunction in the AMD disease process. As a high energy demand organ, the eye is particularly susceptible to the consequences of mitochondrial damage. This damage occurs before vision loss and early intervention targeting the mitochondria function would likely protect or rescue RPE mitochondrial function and therefore attenuate disease progression (The Journal of Neuroscience, May 2015, 7304).

Diabetic retinopathy (DR) is one of the most common microvascular complications of diabetes and remains a major cause of preventable blindness among adults of working age. The prevalence of DR increases with the duration of diabetes, and nearly all patients with type I diabetes and more than 60% with type II diabetes have some degree of retinopathy after 20 years. Current treatment for DR (laser photocoagulation, intravitreal corticosteroids, intravitreal anti-VEGF agents and vitreoretinal surgery) are applicable only at advanced stages of the disease and are associated with significant adverse effects (R. Simo et al., Diabetes Care, 2009, 1556). Therefore, new pharmacological treatments for early stages of the disease are needed.

Diabetic macular edema (DME) is an advanced, vision-limiting complication of DR that affects nearly 30% of patients who have had diabetes for at least 20 years and is responsible for much of the vision loss due to DR. The historic standard of care for DME has been macular laser photocoagulation, which has been shown to stabilize vision and reduce the rate of further vision loss by 50%; however, macular laser photocoagulation leads to significant vision recovery in only 15% of treated patients.

Ophthalmic diseases such as AMD which affect the back of the eye, in particular the retina, choroid and surrounding tissues, are very difficult to treat. Accessibility to the back of eye is one of the main reasons for this difficulty. Currently, most back of the eye or retinal/choroidal diseases are treated using intravitreal injections which deliver a drug into the vitreous adjacent to the retina, or by systemic administration. Intravitreal injections expose the back of the eye to a concentration of the drug that is sufficiently high to be effective to treat the disease. With systemic delivery, however, it is difficult to achieve adequate drug concentrations in the retina and high systemic doses may cause adverse side-effects.

Retinal disorders are a large and diverse group of conditions affecting young and old from many cultures, races and ethnicities. Many ophthalmic disorders are inherited, meaning that they are due to a genetic mutation. An individual can inherit such a mutation, even if they have no clear family history of vision loss. In other instances, many members and generations of a family may experience vision loss.

Retinitis pigmentosa (RP) affects the pediatric and young adult population, and is the leading cause of inherited retinal degeneration-associated blindness. Diabetic retinopathy (DR) is the principal cause of blindness in middle-aged working adults. Stargardt's disease is the most common form of inherited juvenile macular degeneration for which there is currently no treatment. The progressive vision loss associated with Stargardt's disease is caused by the death of photoreceptor cells in the macula. Decreased central vision is a hallmark of Stargardt's disease. Side vision is usually preserved. Stargardt's disease typically develops during childhood and adolescence.

A topical treatment would be a welcome relief to people suffering from retinal diseases such as AMD. The adverse effects associated with intravitreal injections are mainly related to complications with the injection procedure and can include inflammation within the eye (endophthalmitis), increased eye pressure, traumatic cataract and a detached retina. A topical treatment, for example with a cream or with eye drops, would make AMD treatment (both dry and wet) and treatment of other retinal diseases significantly more affordable and available for a larger patient group. The ophthalmic pharmaceutical industry has generally been unable to find drugs that upon topical dosing provide sufficient drug concentration to the back of the eye. Thus, finding a drug that will penetrate the cornea, sclera and/or conjunctiva would be a significant advancement.

There is thus an acute need to find alternative methods of treating ophthalmic disorders, such as AMD (both dry and wet forms), diabetic retinopathy, and Stargardt's disease. In particular, there is a need for alternative drug candidates which are able to treat such diseases and which are capable of accumulating in the retina following topical administration.

A need for suitable drug candidates which may be formulated for topical treatment thus exists. This would make treatment regimens more cost-effective and improve patient compliance, thereby resulting in the availability of treatment for many more patients than at present. A topical treatment would also reduce the adverse effects often associated with systemic treatments, especially for elderly people.

The beneficial effects of fenofibrate in diabetic retinopathy have been demonstrated in two large clinical trials (FIELD and ACCORD-EYE). These trials show that fenofibrate treatment provides a relative reduction in DR progression of 30-40% over 4-6 years, with greater benefit in patients with pre-existing DR. These benefits are achieved despite a lack of significant reduction in triglycerides and small LDL cholesterol which is the main indication for treatment with fenofibrate (A. Ciudin et al., PPAR Res., 2013, 686525).

Fenofibrate activates the peroxisome proliferator activator receptor alpha (PPARα) with an EC₅₀ value in the micromolar range. PPARα is a ligand-activated transcription factor and belongs to the nuclear receptor superfamily. It is highly expressed in the liver, and also in the microvascular, neuronal, and glial tissues of many organs, including the retina. PPARα is an attractive therapeutic target for many diseases and pathological conditions due in part to its antioxidant, anti-inflammatory, and hypolipidemic effects. Interestingly, PPARα is downregulated in the diabetic retina and kidney, and although the regulatory mechanisms responsible for diabetes-induced PPARα downregulation are unclear, decreased PPARα levels may play a pathological role in diabetic microvascular complications. It has also been found that retinal levels of PPARα, but not PPARγ or PPARβ/δ, are decreased in diabetes, suggesting that PPARα plays a more crucial role than other PPARs in repressing development of diabetic retinopathy (Y. Hu et al., Proceedings of the National Academy of Sciences of the United States of America, vol. 110, no. 38, pp. 15401-15406, 2013). A role for PPARα in protective pathways in AMD models has also been highlighted in studies which demonstrated a potent effect of PPARα agonists on inhibiting pathological neovascularization in the retina (Del. V. Cano et al., PPAR Res., 2008, 821592).

PPAR agonists for systemic use have, however, been hampered by serious side effects. For example, the fibrates show a variety of negative pharmacotoxicological profiles. Most fibrates cause some level of myopathy, including pain and creatine phosphokinase release, a consequence of muscle death. Mitochondrial impairment is increasingly implicated in the etiology of toxicity caused by some fibrates and it has been shown that the rank order of mitochondrial impairment accords with clinical adverse events observed with different fibrates (Toxicology and Applied Pharmacology 223 (2007) 277-287). There is thus a need for alternative PPAR agonists which do not impair the mitochondrial function.

Polyunsaturated fatty acids and their metabolites are involved in many physiological and pathophysiological reactions and as such possess a range of important biological activities. They affect plasma lipids and lipid metabolism. They are incorporated into cell membranes where they influence different cell functions. They are also involved in inflammatory diseases and they also influence and control gene expression. However, due to their limited stability in vivo and their lack of biological specificity, PUFAs have not achieved widespread use as therapeutic agents except as triglyceride lowering agents. The only approved indication for polyunsaturated fatty acid derived drugs is the reduction of triglyceride (TG) levels in adult patients with severe (≥500 mg/dL) hypertriglyceridemia (HTG).

The Age-Related Eye Disease Study (AREDS) was designed to determine if daily intake of certain vitamins and minerals could reduce the risk of cataract and advanced age-related macular degeneration (AMD). This study included a placebo-controlled trial, launched in 1992, to evaluate a combination of vitamins E and C, beta-carotene, and zinc—known as the AREDS formulation. In 2001, the investigators reported that the AREDS formulation reduced the risk of advanced AMD by about 25% over a five-year period. There was no effect on cataract. In 2006, the investigators began a separate clinical trial called AREDS2. The primary goal was to determine if adding omega-3 fatty acids or the antioxidants lutein and zeaxanthin to the original AREDS formulation would make it more effective for reducing the risk of advanced AMD or cataract. In the AREDS2 trial, adding DHA/EPA or lutein/zeaxanthin to the original AREDS formulation (containing beta-carotene) had no additional overall effect on the risk of advanced AMD (information from the national eye institute—see: https://nei.nih.gov/areds2/MediaQandA).

In WO 2006/117664 it is suggested that alpha-ethyl DHA derivatives have a combined PPARγ and PPARα effect which may be advantageous to patients with insulin resistance, metabolic syndrome and type II diabetes. In Larsen et al. (Lipids, 2005: 49) alpha alkylation of saturated fatty acids and omega-3 PUFAs is suggested to increase their activation of PPAR receptors compared to their non-alkylated analogues. Alpha-substituted, sulfur or oxygen containing omega-3 PUFAs are also described in WO 2010/128401 and in WO 2010/008299. Some of these compounds are reported to activate PPARα with an EC₅₀ value of 200-400 nM and an efficacy of 80-85% compared to the positive control GW7647 (EC₅₀ 0.45 nM, efficacy 100%). However, none of these earlier documents suggests the use of such compounds in the treatment of any ophthalmic disorder or in retinal diseases such as diabetic retinopathy, AMD, and diabetic macular edema.

Against this background, it has now surprisingly been found that certain fatty acid derivatives as described herein have good PPARα activating properties and are thus particularly suitable for use in the treatment and/or prevention of ophthalmic disorders, in particular retinal diseases such as diabetic retinopathy and AMD. We have also surprisingly found that these derivatives serve to protect against oxidative stress-induced mtDNA damage that has been found to positively correlate with progression of retinal disorders such as AMD (Lin et al., Invest. Ophthalmol. Vis. Sci. 2001, 3521). Whilst not wishing to be bound by theory, since mtDNA damage occurs before vision loss, early intervention to protect or rescue mitochondria function would be expected to attentuate disease progression.

RPE cells are essential for photoreceptor cell survival. When RPE cells are damaged or die, photoreceptor function is impaired, and the photoreceptor cells die as a consequence. Thus, oxidative stress-mediated injury and cell death in RPE cells impairs vision, particularly when the cells of the macula are affected. The macula is the area of the retina responsible for visual acuity. The pathophysiology of many retinal degradations involves oxidative stress leading to apoptosis of RPE cells (Mukherjee, PNAS, 2004, 101, 8491).

We have surprisingly found that compounds of the invention effectively protect RPE cells from apoptosis during conditions of oxidative stress. This observation substantiates the possible use of these compounds in the treatment of degenerative eye diseases (e.g. age related macular degeneration (AMD), Stargardt's disease, retinitis pigmentosa, and glaucoma) and the use in treatment for other ocular diseases caused by oxidative stress (e.g. cataract, dry eye disease, uveitis, etc.).

### SUMMARY OF THE INVENTION

In one aspect the present invention relates to a lipid compound of formula (I), or a pharmaceutically acceptable salt thereof, for the prevention and/or treatment of an ophthalmic disorder, in particular a retinal degenerative disorder or an ocular inflammatory disease:

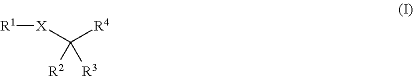

wherein
R¹ is either a C₉ to C₂₂ alkyl group, or a C₉ to C₂₂ alkenyl group having from 1 to 6 double bonds;
R² is selected from the group consisting of a halogen atom, a hydroxy group, an alkyl group, an alkoxy group, an alkylthio group, a carboxy group, an acyl group, an amino group, and an alkylamino group;
R³ is a hydrogen atom, or a group R²;
R⁴ is a carboxylic acid or a derivative thereof; and
X is methylene (—CH₂—), or an oxygen or sulfur atom.

In another aspect the invention relates to a novel omega-6 lipid compound of formula (II), or a pharmaceutically acceptable salt thereof:

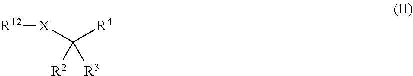

wherein
R¹² is a C₉ to C₂₂ alkenyl group having from 1 to 5 double bonds (e.g. 2 to 5 double bonds) in which:
   the first double bond counting from the ω-end is at carbon 6; and
   where two or more double bonds are present, at least one pair of consecutive double bonds is interrupted by a single methylene group;
R² is selected from the group consisting of a halogen atom, a hydroxy group, an alkyl group, an alkoxy group, an alkylthio group, a carboxy group, an acyl group, an amino group, and an alkylamino group;
R³ is a hydrogen atom, or a group R²;
R⁴ is a carboxylic acid or a derivative thereof; and
X is methylene (—CH₂—), or an oxygen or sulfur atom.

In a further aspect the invention relates to an omega-6 lipid compound of formula (II), or a pharmaceutically acceptable salt thereof for use as a medicament.

In a further aspect the invention relates to an omega-6 lipid compound of formula (II), or a pharmaceutically acceptable salt thereof for the prevention and/or treatment of an ophthalmic disorder, in particular a retinal degenerative disorder or an ocular inflammatory disease.

In a further aspect the invention relates to a process for the preparation of an omega-6 lipid compound of formula (II), or a pharmaceutically acceptable salt thereof.

In a further aspect the present invention provides a pharmaceutical composition comprising an omega-6 lipid compound of formula (II), or a pharmaceutically acceptable salt thereof, together with one or more pharmaceutically acceptable carriers, excipients or diluents.

A further aspect of the present invention relates to a lipid composition comprising an omega-6 lipid compound of formula (II), or a pharmaceutically acceptable salt thereof. The use of such a lipid composition as a medicament, in particular in the prevention and/or treatment of an ophthalmic disorder (e.g. a retinal degenerative disease or an ocular inflammatory disease), forms a further aspect of the invention.

Use of any of the compounds herein described in the manufacture of a medicament for use in the prevention and/or treatment of an ophthalmic disorder, in particular a retinal degenerative disorder or an ocular inflammatory disease, forms a further aspect of the invention.

A method of preventing and/or treating an ophthalmic disorder, in particular a retinal degenerative disorder or an ocular inflammatory disease, said method comprising the step of administering to a patient in need thereof (e.g. a human subject) a pharmaceutically effective amount of any compound as herein described, or a pharmaceutically acceptable salt thereof, forms a yet further aspect of the invention.

### DETAILED DESCRIPTION OF THE INVENTION

Definitions

As used herein, the term “lipid compound” relates to a fatty acid analogue derived from a saturated or unsaturated fatty acid, e.g. from a mono-unsaturated fatty acid, or a poly-unsaturated fatty acid.

As used herein, the term “lipid composition” relates to a composition comprising at least one lipid compound as herein defined, together with one or more naturally occurring or non-naturally occurring lipid components, for example a saturated fatty acid or a mono- or poly-unsaturated fatty acid. It is envisaged that a “lipid compound” as herein described for use according to the invention will form the major component of any lipid composition.

A “pharmaceutically effective amount” relates to an amount that will lead to the desired pharmacological and/or therapeutic effect, i.e. an amount of the agent which is effective to achieve its intended purpose. While individual patient needs may vary, determination of optimal ranges for effective amounts of the active agent is within the capability of one skilled in the art. Generally, the dosage regimen for treating an ophthalmic disorder with any of the compounds described herein is selected in accordance with a variety of factors including the the nature of the medical condition and its severity.

By “a pharmaceutical composition” is meant a composition in any form suitable to be used for a medical purpose.

“Treatment” includes any therapeutic application that can benefit a human or non-human animal (e.g. a non-human mammal). Both human and veterinary treatments are within the scope of the present invention, although primarily the invention is aimed at the treatment of humans. Veterinary treatment includes the treatment of livestock and domestic animals (e.g. pets such as cats, dogs, rabbits, etc.). It also includes the treatment of farmed fish, e.g. salmon. Treatment may be in respect of an existing ophthalmic disorder or it may be prophylactic.

Fatty acids are straight-chained hydrocarbons having a carboxylic acid (—COOH) group at one end, conventionally denoted the α (alpha) end. By convention, the numbering of the carbon atoms starts from the α-end such that the carbon atom of the carboxylic acid group is carbon atom number 1. The α-carbon is the carbon atom in the hydrocarbon chain adjacent to the carbon atom of the —COOH group (i.e. carbon number 2 is the α-carbon). The β (beta) carbon is the next carbon atom along in the hydrocarbon chain (i.e. carbon number 3 is the β-carbon). The other end, which is usually a methyl (—CH₃) group, is conventionally denoted ω (omega) such that the terminal carbon atom is the ω-carbon. Any double bonds present are labelled with cis-/trans-notation or E-/Z-notation, where appropriate.

The term “alpha-substituted” or “alpha-substitution” refers to a substitution at the carbon atom denoted 2 in accordance with the numbering of the carbon chain as described above.

In “ω-x” (omega-x; also sometimes described as n-x) nomenclature a double bond is located on the xth carbon-carbon bond, counting from the terminal carbon (i.e. the ω-carbon, equivalently referred to as the n-carbon) toward the carbonyl carbon. For example, α-linolenic acid is classified as an n-3 or omega-3 (or simply “ω-3”) fatty acid.

As used herein, the term “methylene interrupted double bonds” relates to the case where a methylene group (—CH₂—) is located in between two separate double bonds in a carbon chain of a lipid compound with no other types of functional group located in between these two double bonds. Successive double bonds can be interrupted by one or more than one (e.g. two or three) methylene groups. In certain embodiments, successive or consecutive double bonds are separated by only one methylene group.

As will be understood, the compounds described herein may exist in various stereoisomeric forms, including enantiomers, diastereomers, and mixtures thereof. The invention encompasses all optical isomers of the compounds described herein and mixtures of optical isomers. Hence, compounds that exist as diastereomers, racemates and/or enantiomers are within the scope of the invention.

As used herein, the term “ophthalmic disorder” is to be construed broadly to encompass any disease or condition which affects any part or parts of the eye. The disorder may involve the optic nerve, retina, extraocular eye muscles, eyelids, anterior segment of the eye, posterior segment of the eye, eye surface, or cornea, for example. It may be hereditary meaning that it is due to a genetic mutation, but it need not be. Examples of specific ophthalmic disorders which may be treated or otherwise prevented in accordance with the invention are provided herein. Those conditions affecting the back of the eye, for example the optic nerve or the retina (or parts of the retina, e.g. the macula), are of particular interest.

Compounds Useful in the Invention

The invention is based in part on the discovery that certain lipid compounds are capable of treating and/or preventing ophthalmic disorders, in particular retinal disorders such as diabetic retinopathy and age-related macular degeneration (AMD), and ocular inflammatory disorders.

In one aspect the present invention therefore relates to a lipid compound of formula (I), or a pharmaceutically acceptable salt thereof, for the treatment and/or prevention of an ophthalmic disorder:

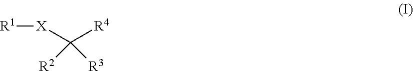

In formula (I):
R¹ is either a C₉ to C₂₂ alkyl group, or a C₉ to C₂₂ alkenyl group having from 1 to 6 double bonds;
R² is selected from the group consisting of a halogen atom, a hydroxy group, an alkyl group, an alkoxy group, an alkylthio group, a carboxy group, an acyl group, an amino group, and an alkylamino group;
R³ is a hydrogen atom, or a group R²;
R⁴ is a carboxylic acid or a derivative thereof; and
X is methylene (—CH₂—), or an oxygen or sulfur atom.

As will be understood from general formula (I), the lipid compounds for use in the invention have at least one alpha-substituent, i.e. R² is other than hydrogen. In some cases two alpha-substituents may be present, i.e. where R³ is a group R². In these cases, the two alpha-substituents may be the same or different, i.e. R³ can be independently selected from any of the groups listed for R². In certain embodiments R² and R³ may be identical. Certain compounds for use in the invention may additionally include a heteroatom at the beta-position, i.e. where X is either an oxygen or sulfur atom. Although not wishing to be bound by theory, alpha-substitution and/or the presence of a beta-heteroatom is considered to improve the metabolic stability of the lipid compounds compared to their corresponding natural fatty acids, e.g. to improve their stability with respect to (3-oxidation. Where R² and/or R³ is a halogen atom, this may be selected from the group consisting of fluorine, chlorine, bromine, and iodine. However, most typically this will be fluorine.

Where R² and/or R³ is an alkyl group this may be straight-chained or branched, preferably a straight-chained or branched C₁₋₆ alkyl. For example, the alkyl group may be selected from methyl, ethyl, n-propyl, isopropyl, n-butyl, sec-butyl, and n-hexyl. Preferred alkyl groups are short-chained, e.g. having 1 to 3 carbon atoms. The alkyl group may be substituted or unsubstituted. Where this is substituted it may be mono- or poly-substituted. Examples of suitable substituents include halogen (e.g. fluorine), hydroxy, thiol, or methoxy, so for example the alkyl group may be selected from among —CF₃, —CH₂CF₃, —CH₂OCH₃, —CH₂OCF₃, and —CH₂CH₂OCH₃. In one embodiment, however, the alkyl group will be unsubstituted. Unsubstituted C₁₋₆ alkyl groups, preferably C₁₋₃ alkyl groups, e.g. methyl and ethyl, are preferred.

Where R² and/or R³ is an alkoxy group, the alkyl component of the alkoxy group may be any alkyl group as defined above. For example, the alkoxy group may be selected from the group consisting of methoxy, ethoxy, propoxy, isopropoxy, sec-butoxy, —OCH₂CF₃, and —OCH₂CH₂OCH₃. It is preferred that the alkyl component is a straight-chained alkyl having from 1 to 6 carbons, e.g. 1 to 3 carbons. Methoxy and ethoxy are particularly preferred.

Where R² and/or R³ is an alkylthio group, the alkyl component of said alkylthio group is preferably an alkyl group as defined above. For example, the alkylthio group may be selected from the group consisting of methylthio, ethylthio, and isopropylthio.

Where R² and/or R³ is an acyl group, the alkyl component of the acyl group may be any alkyl group as defined above. Typically, this will be an unsubstituted, straight-chained alkyl group. For example, the acyl group may be a group of the formula —C(O)C₁₋₆ alkyl, e.g. —C(O)CH₂CH₃ or —C(O)CH₃.

Where R² and/or R³ is an alkylamino group this may be a group of the formula —NHR′, or a group of the formula —NR′₂ wherein each R′ is independently a C₁₋₃ alkyl group, e.g. methyl. For example, the alkylamino group may be selected from the group consisting of methylamino, dimethyl amino, ethylamino, and diethylamino.

In an embodiment the compounds for use in the invention include a single alpha-substituent, i.e. R³ is a hydrogen atom.

In an embodiment R³ is hydrogen, and X is selected from oxygen and sulfur. Such compounds include both an alpha-substituent and a beta-heteroatom and may be represented by the general formulae (Ia) and (Ib):

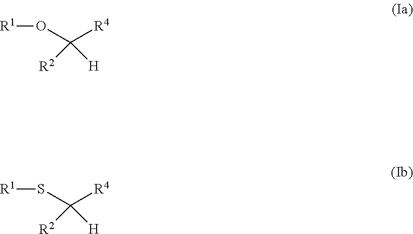

In formula (Ia) and (Ib), R¹, R² and R⁴ are as described herein. Preferably R² is a fluorine atom, optionally substituted C₁₋₃ alkyl (e.g. methyl, ethyl, —CF₃ or —CH₂CF₃), C₁₋₃ alkoxy (e.g. methoxy or ethoxy), C₁₋₃ alkylthio (e.g. —SCH₃ or —SCH₂CH₃), an amino group, or an alkylamino group of the formula —NR′₂ where each R′ is independently a hydrogen atom or a C₁₋₃ alkyl group (e.g. methylamino or dimethyl amino). More preferably, R² is methyl or ethyl.

In an embodiment R³ is hydrogen and X is —CH₂—. In such compounds R¹, R² and R⁴ are as described herein. Preferably R² is a hydroxy group, an optionally substituted C₁₋₃ alkyl (e.g. methyl, ethyl, —CF₃ or —CH₂CF₃), C₁₋₃ alkoxy (e.g. methoxy or ethoxy), C₁₋₃ alkylthio (e.g. —SCH₃ or —SCH₂CH₃), a carboxy group, an acyl group (e.g. —C(O)CH₃), an amino group, or an alkylamino group of the formula —NR′₂ where each R′ is independently a hydrogen atom or a C₁₋₃ alkyl group (e.g. methylamino or dimethyl amino). Preferably, R² is C₁₋₃ alkyl, e.g. methyl or ethyl, or C₁₋₃ alkoxy, e.g. methoxy or ethoxy.

In an embodiment the compounds for use in the invention include two alpha-substituents, i.e. R³ is other than hydrogen. In this embodiment, R² and R³ may be the same or different. Preferably they will be same and may, for example, both represent an unsubstituted C₁₋₃ alkyl, e.g. methyl or ethyl.

The substituent R⁴ is a carboxylic acid (—COOH) group or a derivative thereof. Suitable derivatives include a carboxylic ester, a carboxylic anhydride, a carboxamide, a monoglyceride, a diglyceride, a triglyceride, and a phospholipid.

Where R⁴ is a carboxylic ester then the compounds for use in the invention may be a compound of formula (I) in which R⁴ is a group of the formula —COOR⁵ where R⁵ is an alkyl group as defined herein, preferably a C₁₋₆ alkyl group. Preferably R⁴ may be selected from the group consisting of ethyl carboxylate, methyl carboxylate, n-propyl carboxylate, isopropyl carboxylate, n-butyl carboxylate, sec-butyl carboxylate, and n-hexyl carboxylate.

Where R⁴ is a carboxylic anhydride then the compounds for use in the invention may be a compound of general formula (III):

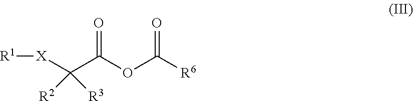

wherein

R¹, R², R³ and X are as defined herein; and

R⁶ is an alkyl or alkoxy group as defined herein, preferably a C₁₋₆ alkyl or C₁₋₆ alkoxy group.

In a further embodiment where R⁴ is a carboxylic anhydride the compounds for use in the invention may be a compound of formula (IV):

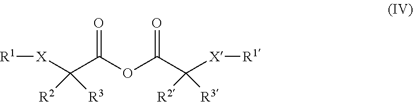

wherein

R^{1′}, R^{2′}, R^{3′} and X′ are respectively chosen from among the same groups as R¹, R², R³ and X as herein defined. In this embodiment R¹, R², R³ and X may respectively be the same as or different to R^{1′}, R^{2′}, R^{3′} and X′. In one embodiment of the compounds of formula (IV), R¹,

R², R³ and X, respectively, may be identical to R^{1′}, R^{2′}, R^{3′} and X′.

Where R⁴ is a carboxamide then the compound of formula (I) may be a compound of formula (V):

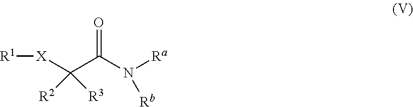

(wherein

R¹, R², R³ and X are as defined herein; and

R^{a} and R^{b} are independently selected from hydrogen and C₁₋₃ alkyl (e.g. methyl)).

Where R⁴ is a carboxamide group, this may be selected from the group consisting of N-methyl carboxamide, N,N-dimethyl carboxamide, N-ethyl carboxamide, and N,N-diethyl carboxamide.

Where R⁴ is a monoglyceride the compounds for use in the invention may be selected from the following compounds of formula (VI) and (VII):

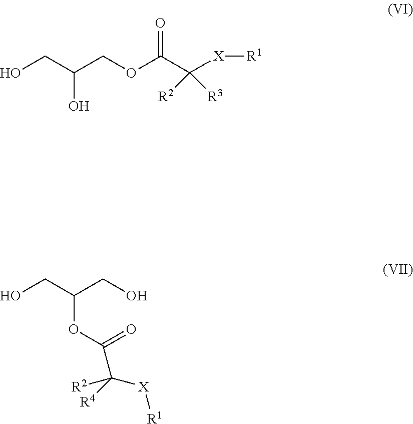

wherein R¹, R², R³ and X are as defined herein.

Where R⁴ is a diglyceride the compounds for use in the invention may be selected from the following compounds of formula (VIII) and (IX):

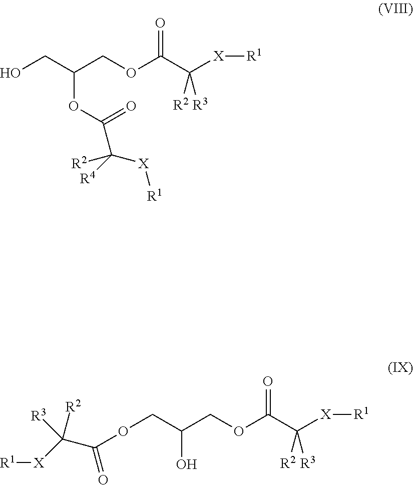

wherein R¹, R², R³ and X are as defined herein. In either of these compounds identically labelled substituents may be the same or different, although typically these will be identical to one another.

Where R⁴ is a triglyceride the compounds for use in the invention may be a compound of formula (X):

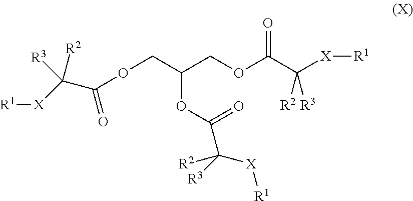

wherein R¹, R², R³ and X are as defined herein. In the compound of formula (X) identically labelled substituents may be the same or different, although typically these will be identical to one another.

Where the compound of formula (I) is a phospholipid, such compounds may be represented by formulae (XI), (XII) and (XIII):

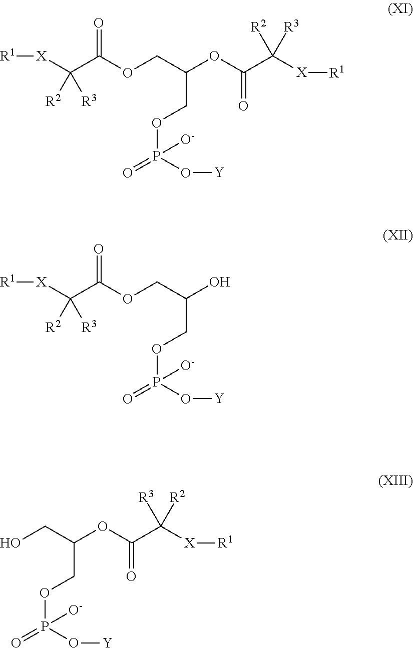

wherein R¹, R², R³ and X are as defined herein, and wherein Y is selected from the following:

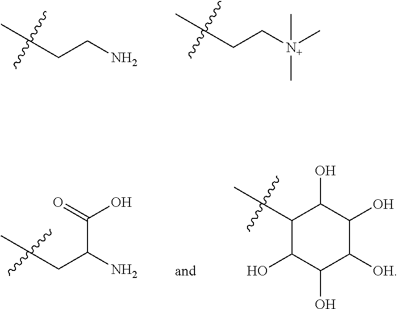

In the compound of formula (XI) identically labelled substituents may be the same or different, although typically these will be identical to one another.

In any of the lipid compounds for use in the invention R¹ is either a C₉ to C₂₂ alkyl group, or a C₉ to C₂₂ alkenyl group having from 1 to 6 double bonds.

In an embodiment of the invention R¹ is a C₉ to C₂₂ alkyl group. In this embodiment, the compounds will typically be derived from a saturated fatty acid. The alkyl group may be straight-chained or branched, although straight-chained C₉ to C₂₂ alkyl groups are generally preferred.

In certain embodiments the group R¹ is a C₁₀ to C₂₂ alkyl group, preferably a C₁₂ to C₂₀ alkyl group, more preferably a C₁₂ to C₁₆ alkyl group, e.g. a C₁₄ alkyl group. Such groups may be straight-chained or branched, however these will typically be straight-chained. Shorter chain alkyl groups, such as C₁₂ to C₁₆ alkyl groups, are generally preferred.

Where R¹ is a C₉ to C₂₂ alkyl group, it is preferred that X is oxygen or sulphur.

In one embodiment where R¹ is a C₉ to C₂₂ alkyl group, X may be oxygen or sulphur, and R² may be a C₁₋₆ alkyl group, e.g. a C₁₋₃ alkyl. An example of such a compound is α-methyl TTA:

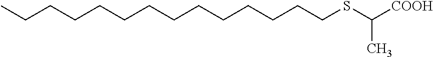 [SMILES: CCCCCCCCCCCCCCSC(C)C(=O)O]

R¹: C₁₄ alkyl

X: —S—

R²: —CH₃

R³: H

R⁴: —CO₂H

In another embodiment the substituent R¹ is a C₉ to C₂₂ alkenyl group having from 1 to 6 double bonds. In this embodiment, the compound will typically be derived from a mono- or poly-unsaturated fatty acid. The alkenyl group may be straight-chained or branched, although straight-chained C₉ to C₂₂ alkenyl groups are generally preferred.

In certain embodiments the group R¹ is a C₁₀ to C₂₂ alkenyl group, preferably a C₁₂ to C₁₉ alkenyl group, e.g. a C₁₉ alkenyl group, more preferably a C₁₄ to C₁₈ alkenyl group, e.g. a C₁₅ or C₁-alkenyl group. Such groups may be straight-chained or branched, however these will typically be straight-chained. Shorter chain alkenyl groups, such as C₁₂ to C₁₅, C₁₃ to C₁₇ or C₁₄ to C₁₈ alkenyl groups, are generally preferred. However, in certain embodiments the group R¹ is a C₁₉ to C₂₂ alkenyl group, e.g. a C₂₀ alkenyl group.

Preferably R¹ is a straight-chained C₉ to C₂₂ alkenyl group. In one embodiment R¹ is a straight-chained ω-3 C₉ to C₂₂ alkenyl group having from 1 to 6 double bonds. In another embodiment R¹ is a straight-chained ω-6 C₉ to C₂₂ alkenyl group having from 1 to 5 double bonds.

Where R¹ is an alkenyl group this may have from 1 to 6 double bonds, preferably from 2 to 4 double bonds, e.g. 2, 3 or 4 double bonds. Where more than one double bond is present it is preferred that at least one pair of successive double bonds is interrupted by no more than one methylene group. In an embodiment each pair of consecutive double bonds is interrupted by no more than one methylene group. Each double bond may independently be in the E-configuration or the Z-configuration. Preferably, where more than one double bond is present, all double bonds are in the same configuration; particularly preferably all are in the Z-configuration. Thus in an embodiment R¹ may be a C₉ to C₂₂ alkenyl group with 2 to 6, preferably 2 to 4, e.g. 2, 3 or 4, double bonds in the Z-configuration where at least one pair of successive double bonds is interrupted by a single methylene group.

In an embodiment X is —CH₂— and R¹ is a C₉ to C₂₂ alkenyl having 2 to 6 double bonds, e.g. a C₁₉, C₁₇, or C₁₅ alkenyl having 3, 4, 5 or 6 double bonds.

In an embodiment X is S or O and R¹ is a C₉ to C₂₂ alkenyl having 2 to 6 double bonds, e.g. a C₂₂, C₂₀, C₁₈, or C₁₅ alkenyl having 3, 4, 5 or 6 double bonds.

Where 2 or more double bonds are present in group R¹ the compounds for use according to the invention may be derived from known polyunsaturated fatty acids (PUFAs). Particularly preferably the compound for use in the invention is either an ω-3 PUFA derivative or an ω-6 PUFA derivative. Such derivatives may typically retain the structure of group R¹ (i.e. retain the same chain length, number and position of double bonds, and E/Z bond configuration) as found in the parent molecule (i.e. PUFA) but are derivatised in that groups X, R², R³ and/or R⁴ differ from those normally found in the PUFA.

Examples of PUFAs from which the compounds disclosed herein may be derived include (all-Z)-4,7,10,13,16,19-docosahexaenoic acid (omega-3 docosahexaenoic acid or omega-3 DHA), (all-Z)-5,8,11,14,17-eicosapentaenoic acid (omega-3 eicosapentaenoic acid or omega-3 EPA), (all-Z)-7,10,13,16,19-docosapentaenoic acid (omega-3 docosapentaenoic acid or omega-3 DPA), (all-Z)-9,12,15-octadecatrienoic acid (omega-3 α-linoleic acid or omega-3 ALA), (all-Z)-5,8,11,14-icosatetraenoic acid (omega-6 arachidonic acid or omega-6 AA), (all-Z)-4,7,10,13,16-docosapentaenoic acid (omega-6 docosapentaenoic acid or omega-6 DPA), (all-Z)-8,11,14-eicosatrienoic acid (omega-6 dihomo-γ-linolenic acid or omega-6 DGLA) and (all-Z)-9,12-octadecadienoic acid (omega-6 linoleic acid or omega-6 LA).

In an embodiment R¹ is a C₉ to C₂₂ alkenyl having 2 to 6 double bonds, e.g. a C₁₉ alkenyl with 6 double bonds, preferably methylene interrupted double bonds in the Z-configuration (e.g. derived from omega-3 DHA), a C₁₇ alkenyl with 5 double bonds, preferably methylene interrupted double bonds in the Z-configuration (e.g. derived from omega-3 EPA); a C₁₅ alkenyl with 3 double bonds, preferably methylene interrupted double bonds in the Z-configuration (e.g. derived from omega-3 alpha-linolenic acid); a C₁₉ alkenyl with 6 double bonds, preferably methylene interrupted double bonds in the Z-configuration (e.g. derived from omega-3 DHA), a C₁₉ alkenyl with 5 double bonds, preferably methylene interrupted double bonds in the Z-configuration (e.g. derived from omega-3 DPA); a C₁₅ alkenyl with 4 double bonds, preferably methylene interrupted double bonds in the Z-configuration (e.g. derived from degradation of EPA) or 3 methylene interrupted double bonds in the Z-configuration and one double bond in the E-configuration (e.g. derived from degradation of EPA); or a C₁₈ alkenyl with 5 double bonds, preferably methylene interrupted double bonds in the Z-configuration or 4 methylene interrupted double bonds in the Z-configuration and one double bond in the E-configuration (e.g. both derived from degradation of DHA).

In another embodiment R¹ is a C₁₇ alkenyl with 4 double bonds, preferably methylene interrupted double bonds in the Z-configuration (e.g. derived from omega-6 AA); a C₁₉ alkenyl with 5 double bonds, preferably methylene interrupted double bonds in the Z-configuration (e.g. derived from omega-6 DPA); a C₁₅ alkenyl with 2 double bonds, preferably methylene interrupted double bonds in the Z-configuration (e.g. derived from omega-6 linoleic acid); a C₁₅ alkenyl with 3 double bonds, preferably methylene interrupted double bonds in the Z-configuration (e.g. derived from degradation of AA).

In an embodiment R¹ may be a C₁₅ alkenyl with 3 or 4 methylene interrupted double bonds in the Z-configuration, a C₁₈ alkenyl with 3 to 5 double bonds, e.g. a C₁₈ alkenyl with 5 methylene interrupted double bonds in the Z-configuration; a C₁₄ to C₂₂ alkenyl group with at least one double bond in the Z-configuration, and having the first double bond at the third carbon-carbon bond from the ω-end of the carbon chain; a C₁₄ to C₂₂ alkenyl group with at least one double bond in the Z-configuration, and having the first double bond at the sixth carbon-carbon bond from the ω-end of the carbon chain; a C₁₈ alkenyl with 3 methylene interrupted double bonds in the Z-configuration; a C₂₀ alkenyl with 5 methylene interrupted double bonds in the Z-configuration; or a C₂₂ alkenyl with 6 methylene interrupted double bonds in the Z-configuration; a C₂₀ alkenyl with 4 methylene interrupted double bonds in the Z-configuration; a C₂₂ alkenyl with 5 methylene interrupted double bonds in the Z-configuration; or a C₁₈ alkenyl with 2 methylene interrupted double bonds in the Z-configuration.

In one embodiment the substituent R¹ is a C₉ to C₂₂ alkenyl having 2 to 6 double bonds, and the compound of formula (I) is derived from an ω-3 or ω-6 polyunsaturated fatty acid; R² and R³ are as herein defined, preferably R² is an alkyl group and R³ is hydrogen; and R⁴ is a carboxylic acid in the form of a free acid.

In one embodiment the lipid compound of formula (I) is an ω-6 lipid compound wherein R¹ is a C₉ to C₂₂ alkenyl group having 1 to 5 double bonds as herein defined and further wherein the first double bond counting from the ω-end is at carbon 6; and R², R³, R⁴ and X are as defined herein.

Preferred ω-6 lipid compounds for use in the invention are those in which R¹ has 2 to 4 double bonds, e.g. 2, 3 or 4 double bonds. In certain embodiments such ω-6 lipid compounds may be derivatives of arachidonic acid or linoleic acid.

In certain embodiments, the lipid compound of formula (I) for use in the invention is an ω-6 lipid compound wherein R¹ is a C₁₅ to C₂₀ alkenyl group having 2 to 4 double bonds.

Particularly preferably, the ω-6 lipid compounds for use in the invention have 2 to 4 double bonds as described above which are each methylene-interrupted, i.e. successive double bonds in the alkenyl chain are separated only by —CH₂— groups.

Particularly preferably, where the ω-6 lipid compounds for use in the invention have 2 to 4 double bonds, the double bonds are all in the Z-configuration.

In an embodiment, where the ω-6 lipid compounds for use in the invention have 2 to 4 double bonds, the pairs of successive double bonds are each methylene-interrupted and are all in the Z-configuration.

In certain embodiments the ω-6 lipid compounds according to the invention are those in which X is either oxygen or sulphur and R³ is hydrogen. In an embodiment the ω-6 lipid compounds according to the invention are those in which X is either oxygen or sulphur, R³ is hydrogen and R¹ is a C₉ to C₂₂ alkenyl having 2 to 5 double bonds, e.g. a C₂₀, C₁₈, or C₁₅ alkenyl having 2, 3 or 4 double bonds.

In certain embodiments the ω-6 lipid compounds according to the invention are those in which X is —CH₂— and R³ is hydrogen. In an embodiment the ω-6 lipid compounds according to the invention are those in which X is —CH₂—, R³ is hydrogen and R¹ is a C₉ to C₂₂ alkenyl having 2 to 5 double bonds, e.g. a C₁₉, C₁₇, or C₁₅ alkenyl having 2, 4 or 5 double bonds.

In one embodiment the ω-6 lipid compounds for use in the invention include those where R¹ is a C₉ to C₂₂ alkenyl group, X is —CH₂— and R² is a C₁₋₆ alkyl group, e.g. a C₁₋₃ alkyl. An example of such a compound is α-ethyl AA:

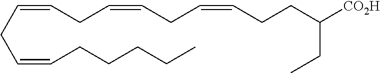 [SMILES: CCCCC/C=C\C/C=C\C/C=C\C/C=C\CCC(CC)C(=O)O]

R¹: C₁₇ alkenyl having four double bonds

X: —CH₂—

R²: —CH₂CH₃

R³: H

R⁴: —CO₂H

In further embodiments of the invention the lipid compound of formula (I) for use according to the invention is an ω-3 lipid compound wherein R¹ is a C₉ to C₂₂ alkenyl group having 1 to 6 double bonds as herein defined and further wherein the first double bond counting from the ω-end is at carbon 3; and R², R³, R⁴ and X are as defined herein.

Preferred ω-3 lipid compounds for use in the invention are those in which R¹ has 3 to 6 double bonds, preferably 3 to 5 double bonds, e.g. 3, 4 or 5 double bonds.

In certain embodiments, the lipid compound of formula (I) for use in the invention is an ω-3 lipid compound wherein R¹ is a C₁₅ to C₂₀ alkenyl group having 2 to 5 double bonds, e.g. 3, 4 or 5 double bonds.

Particularly preferably, the ω-3 lipid compounds for use in the invention have 2 to 5 double bonds as described above which are each methylene-interrupted, i.e. successive double bonds in the alkenyl chain are separated only by —CH₂— groups.

Particularly preferably, where the ω-3 lipid compounds for use in the invention have 2 to 5 double bonds, the double bonds are all in the Z-configuration.

In an embodiment, where the ω-3 lipid compounds for use in the invention have 2 to 5 double bonds, the double bonds are each methylene-interrupted and are all in the Z-configuration.

In certain embodiments the ω-3 lipid compounds for use in the invention are those in which X is either oxygen or sulphur and R³ is hydrogen. In an embodiment the ω-3 lipid compounds for use in the invention are those in which X is either oxygen or sulphur, R³ is hydrogen and R¹ is a C₉ to C₂₂ alkenyl having 2 to 6 double bonds, e.g. a C₂₂, C₂₀, C₁₈, or C₁₅ alkenyl having 3, 4, 5 or 6 double bonds.

In certain embodiments the ω-3 lipid compounds for use in the invention are those in which X is —CH₂— and R³ is hydrogen. In an embodiment the ω-3 lipid compounds for use in the invention are those in which X is —CH₂—, R³ is hydrogen and R¹ is a C₉ to C₂₂ alkenyl having 2 to 6 double bonds, e.g. a C₁₉, C₁₇, or C₁₅ alkenyl having 3, 4, 5 or 6 double bonds.

In certain embodiments, the lipid compound for use in the invention is an ω-3 lipid compound wherein R¹ is a C₁₅ to C₁₈ alkenyl group with 2 to 5 double bonds, e.g. 4 double bonds.

The lipid compounds for use in the invention may be provided in the form of a pharmaceutically acceptable salt. Suitable salts are well known to those skilled in the art and include, but are not limited to, the lithium, sodium, potassium, ammonium, meglumine, tris(hydroxymethyl)aminomethane, diethylamine, arginine, ethylenediamine, piperazine and chitosan salts.

Examples of suitable omega-6 lipid compounds for use in the invention include the following and their pharmaceutically acceptable salts:

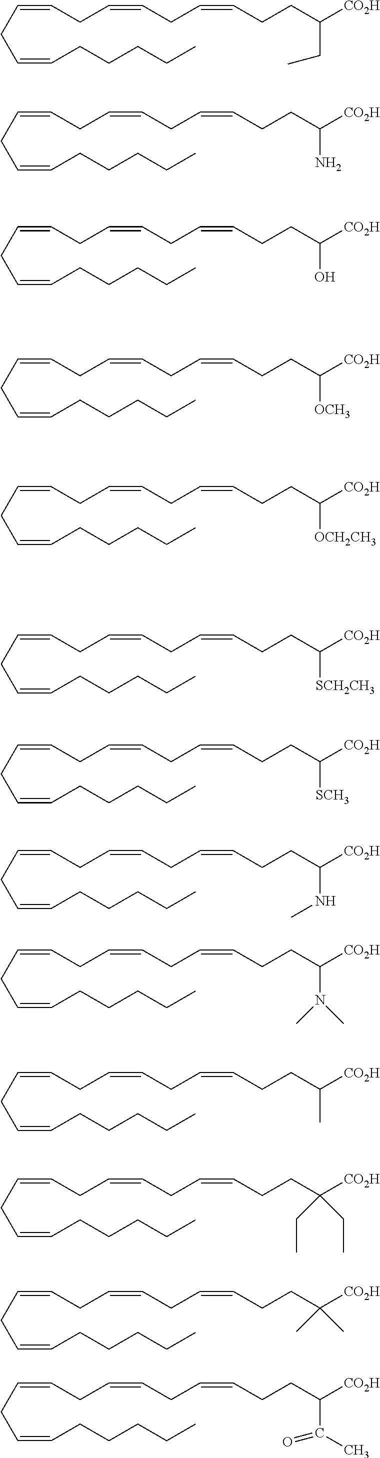

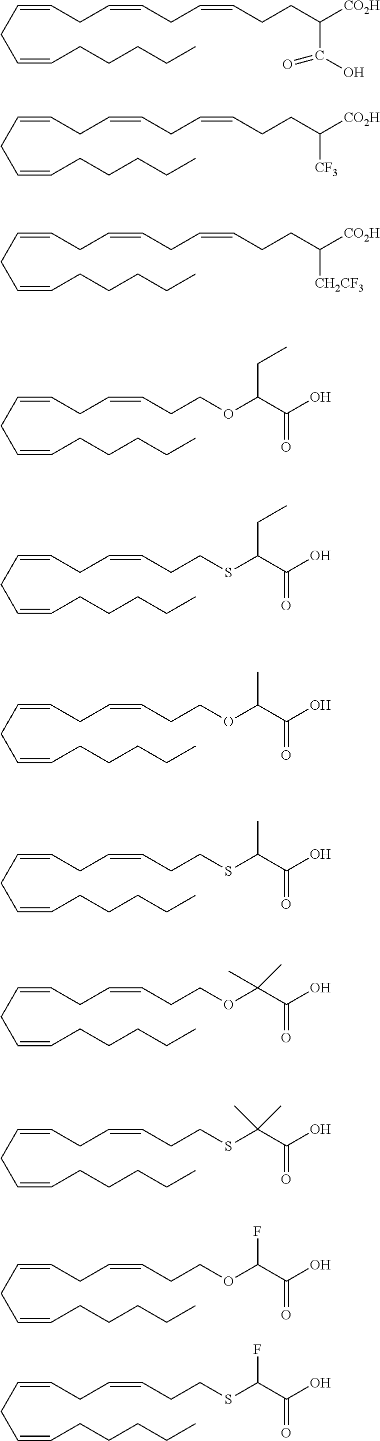

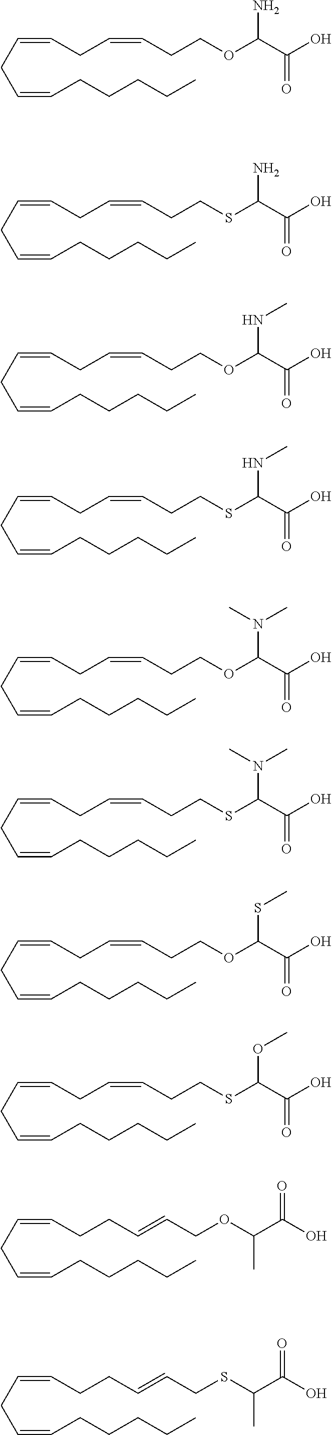

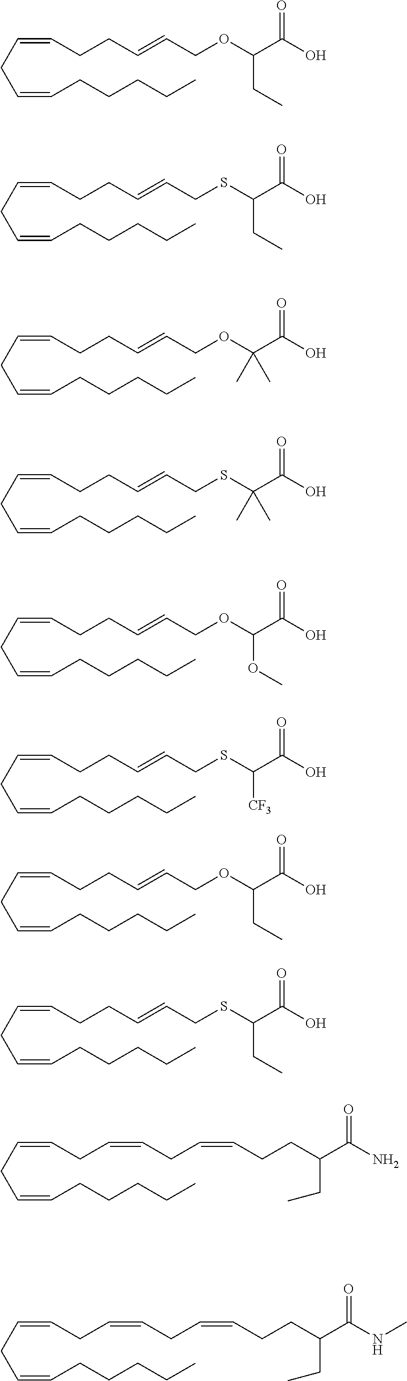

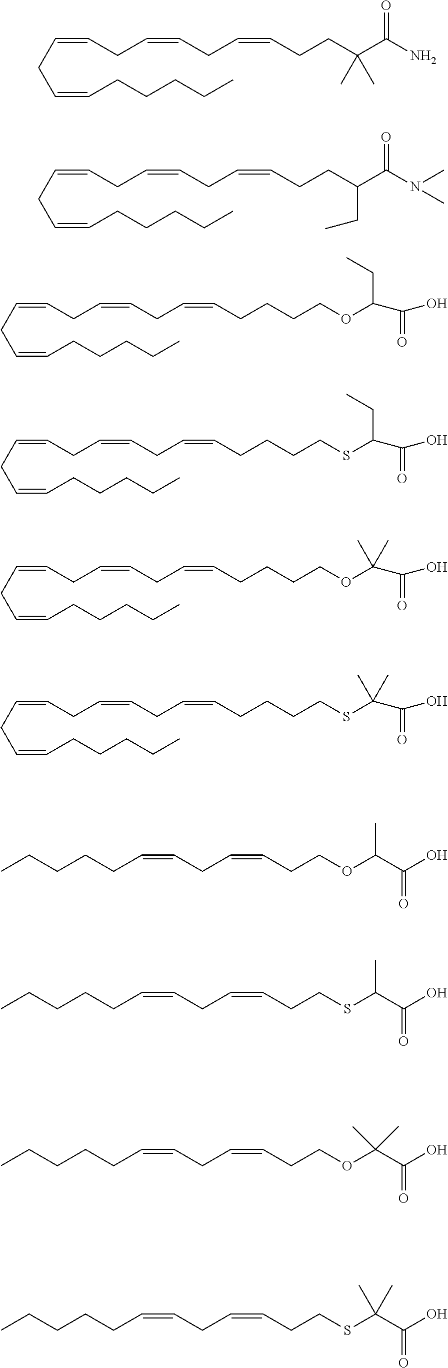

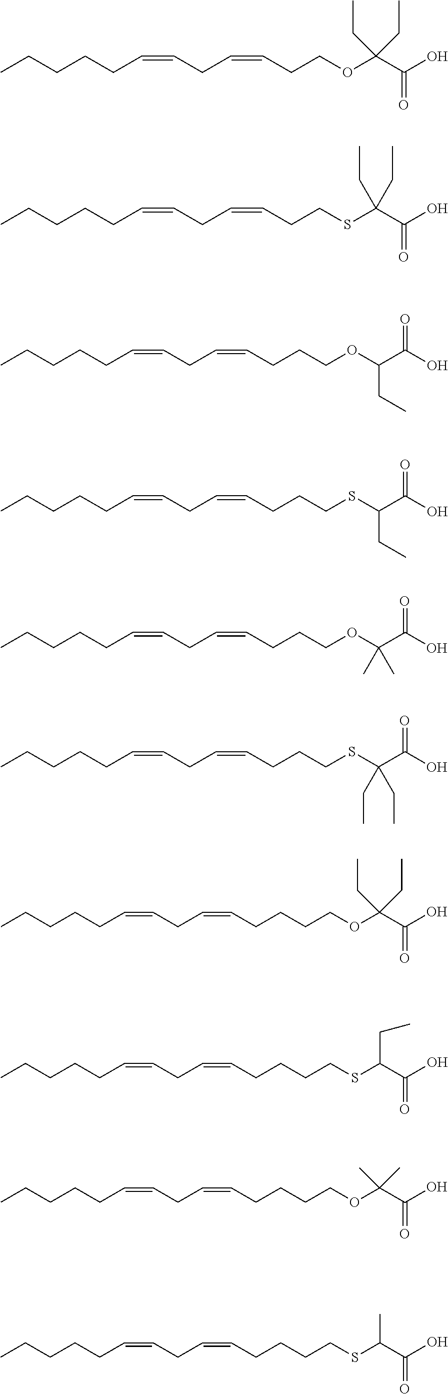

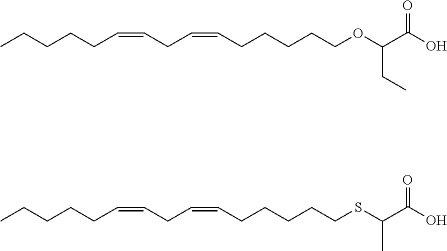 [SMILES: CCCCC/C=C\C/C=C\CCCCCOC(CC)C(=O)O ; CCCCC/C=C\C/C=C\CCCCCSC(C)C(=O)O]

Examples of suitable omega-3 lipid compounds for use in the invention include the following and their pharmaceutically acceptable salts:

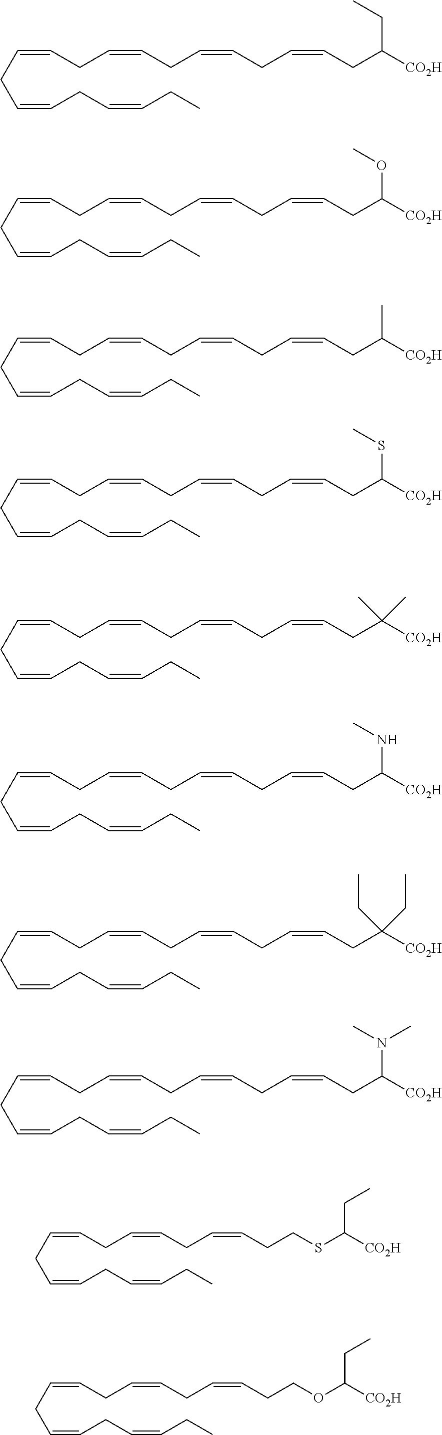

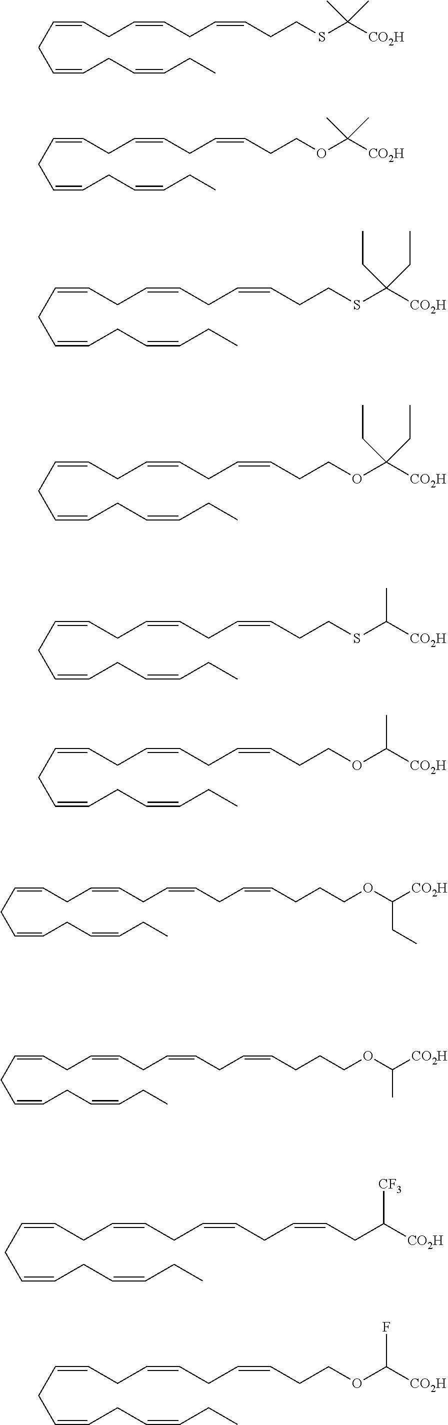

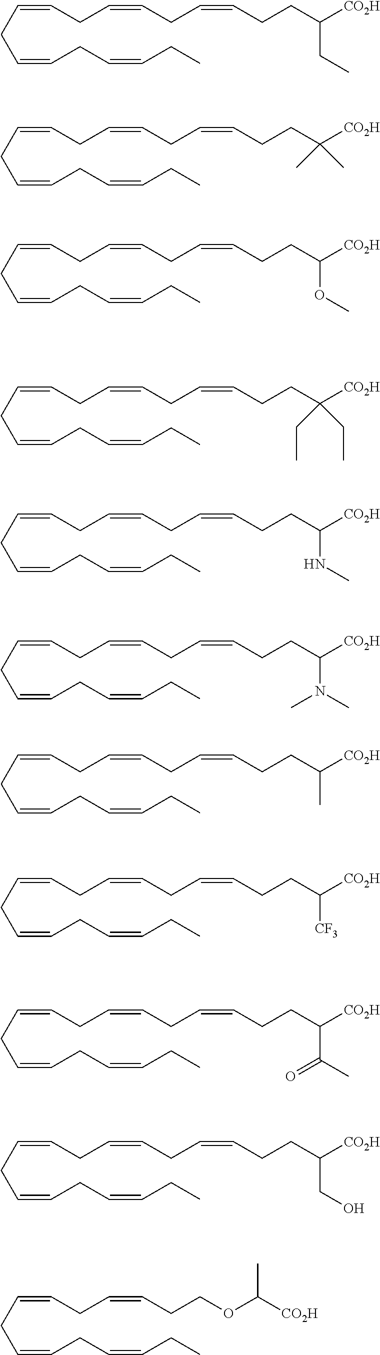

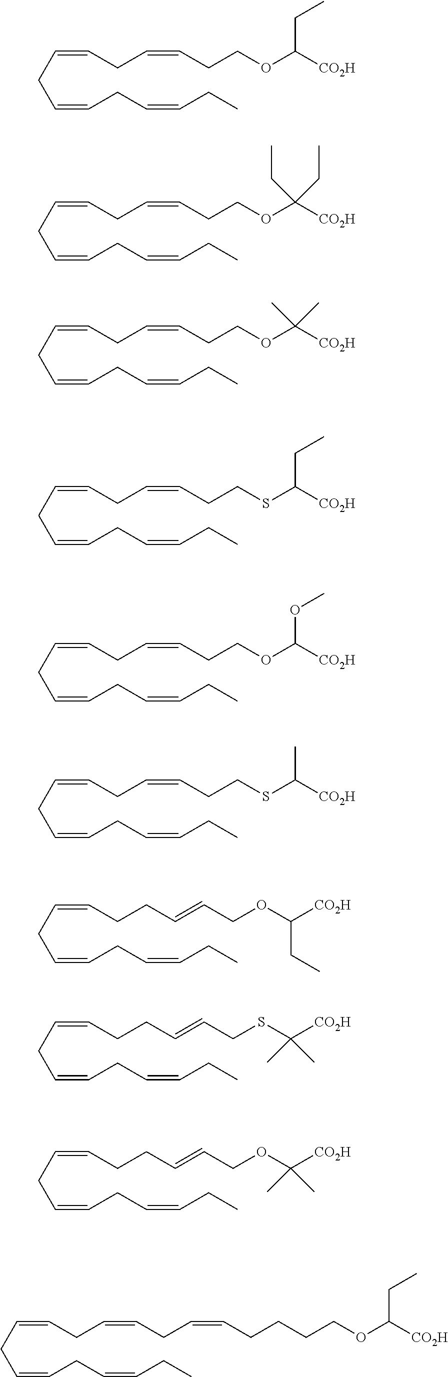

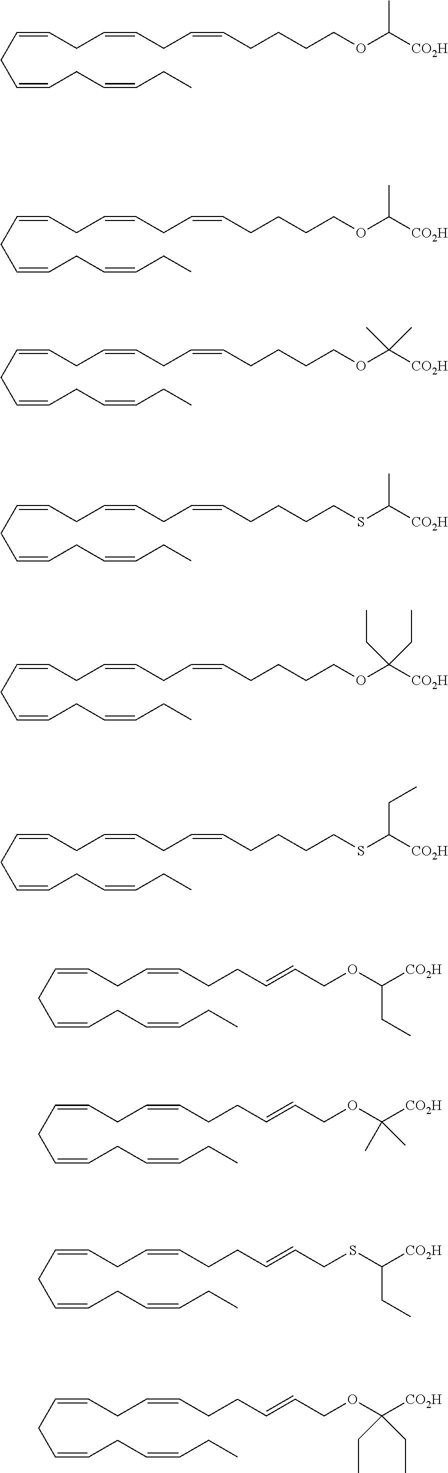

Examples of suitable compounds for use in the invention in which R¹ is an alkyl group include the following and their pharmaceutically acceptable salts:

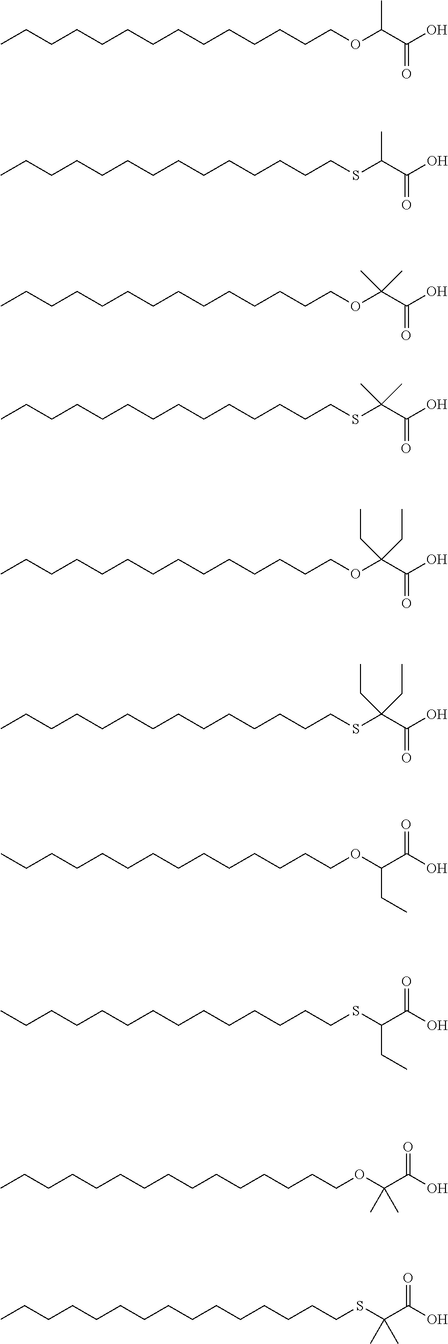

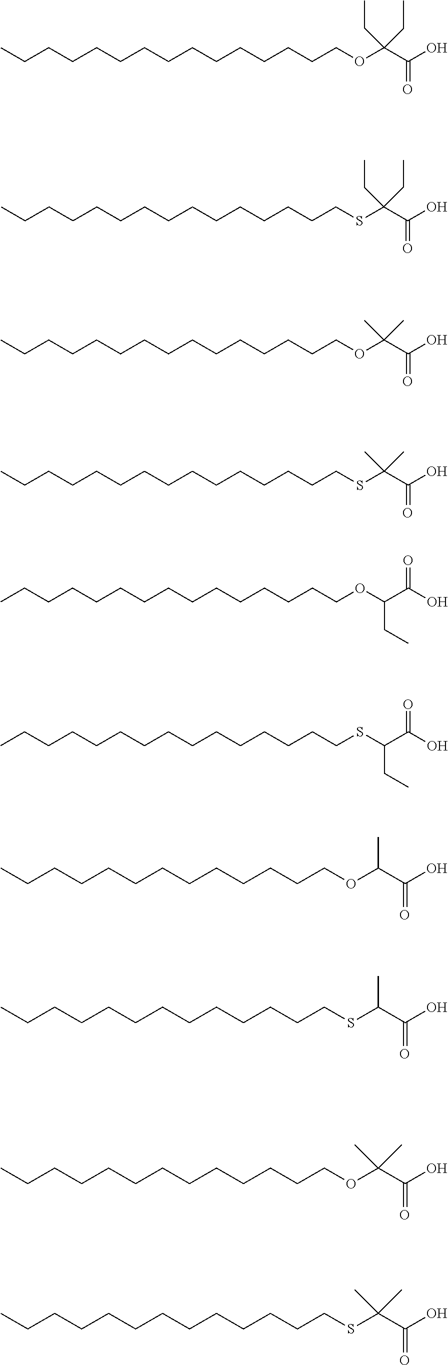

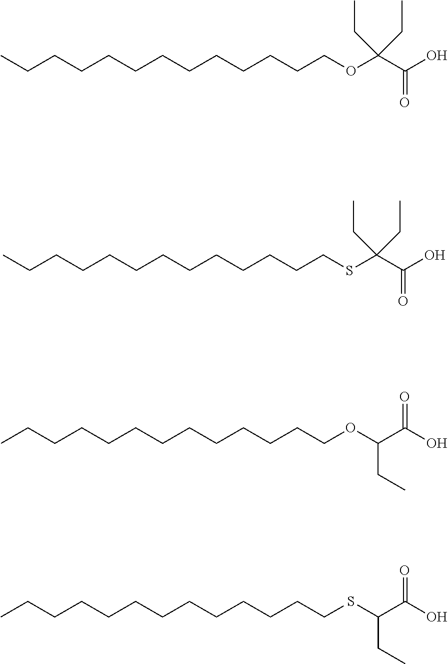 [SMILES: CCCCCCCCCCCCCOC(CC)(CC)C(=O)O ; CCCCCCCCCCCCCSC(CC)(CC)C(=O)O ; CCCCCCCCCCCCCOC(CC)C(=O)O ; CCCCCCCCCCCCCSC(CC)C(=O)O]

Compounds of the Invention

Certain of the omega-6 compounds described herein are novel and these form a further aspect of the invention. Thus, in a further aspect, the present invention provides an omega-6 lipid compound of formula (II), or a pharmaceutically acceptable salt thereof:

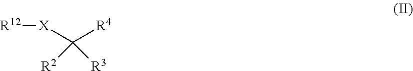

wherein
R¹² is a C₉ to C₂₂ alkenyl group having from 1 to 5 double bonds, preferably 2 to 5 double bonds, in which:
   the first double bond counting from the ω-end is at carbon 6; and
   where two or more double bonds are present, at least one pair of consecutive double bonds is interrupted by a single methylene group;
R² is selected from the group consisting of a halogen atom, a hydroxy group, an alkyl group, an alkoxy group, an alkylthio group, a carboxy group, an acyl group, an amino group, and an alkylamino group;
R³ is a hydrogen atom, or a group R²;
R⁴ is a carboxylic acid or a derivative thereof; and
X is methylene (—CH₂—), or an oxygen or sulfur atom.

Any of the omega-6 compounds described herein with reference to the medical use aspect of the invention represent preferred embodiments of the compounds of the invention. In formula (II), R², R³, R⁴ and X may thus correspond, respectively, to R², R³, R⁴ and X in any of the embodiments described above relating to the medical use of the lipid compounds. As will be understood, the group R¹² in formula (II) may correspond to any of the groups R¹ described herein in the case where R¹ is an alkenyl group and subject to the further requirements that the first double bond counting from the ω-end is at carbon 6 and that where two or more double bonds are present at least one pair of consecutive double bonds is interrupted by a single methylene group.

Preferred ω-6 lipid compounds according to the invention are those of formula (II) in which R¹² has 2 to 5 double bonds, e.g. 2 to 4 double bonds.

In certain embodiments, the compound of formula (II) is an omega-6 lipid compound wherein R¹² is a C₁₅ to C₂₀ alkene with 2 to 4 double bonds.

Particularly preferably, the ω-6 lipid compounds of formula (II) have 2 to 4 double bonds as described above and all of said double bonds are methylene-interrupted, i.e. successive double bonds in the alkenyl chain are separated only by —CH₂— groups, preferably by no more than one —CH₂— group.

Particularly preferably, where the ω-6 lipid compounds of formula (II) have 2 to 4 double bonds, these are all in the Z-configuration.

In an embodiment, where the ω-6 lipid compounds of formula (II) have 2 to 4 double bonds, the double bonds are methylene-interrupted and are all in the Z-configuration.

The omega-6 lipid compounds of formula (II) according to the invention may be provided in the form of a free carboxylic acid (—COOH), or a derivative thereof, such as a carboxylic ester, a carboxylic anhydride, a carboxamide, a monoglyceride, a diglyceride, a triglyceride or a phospholipid as described above.

The omega-6 lipid compounds of formula (II) according to the invention may further be provided in the form of a pharmaceutically acceptable salt such as a lithium, sodium, potassium, ammonium, meglumine, tris(hydroxymethyl)aminomethane, diethylamine, arginine, ethylenediamine, piperazine or chitosan salt.

In a further aspect the present invention provides an omega-6 lipid compound of formula (II), or a pharmaceutically acceptable salt thereof, for use as a medicament.

In a further aspect the present invention provides an omega-6 lipid compound of formula (II), or a pharmaceutically acceptable salt thereof, for the prevention and/or treatment of an ophthalmic disorder.

In a further aspect the present invention provides a pharmaceutical composition comprising an omega-6 lipid compound of formula (II), together with one or more pharmaceutically acceptable carriers, excipients or diluents.

A further aspect of the present invention relates to a lipid composition comprising an omega-6 lipid compound of formula (II). The lipid composition may comprise in the range of 60 to 100% by weight of the omega-6 lipid compounds of formula (II), all percentages by weight being based on the total weight of the lipid composition. For example, at least 60%, at least 70%, at least 80%, or at least 95% by weight of the lipid composition is comprised of omega-6 lipid compounds of formula (II). The lipid composition may further comprise a pharmaceutically acceptable antioxidant, e.g. tocopherol.

The invention further provides a lipid composition comprising an omega-6 lipid compound of formula (II) for use as a medicament.

The invention further provides a lipid composition comprising an omega-6 lipid compound of formula (II) for the treatment and/or prevention of an ophthalmic disorder as herein described.

Use of an omega-6 lipid compound of formula (II) in the manufacture of a medicament for use in the prevention and/or treatment of an ophthalmic disorder, in particular a retinal degenerative disorder or an ocular inflammatory disease, forms a further aspect of the invention.

A method of preventing and/or treating an ophthalmic disorder, in particular a retinal degenerative disorder or an ocular inflammatory disease, said method comprising the step of administering to a patient in need thereof (e.g. a human subject) a pharmaceutically effective amount of an omega-6 lipid compound of formula (II), or a pharmaceutically acceptable salt thereof, forms a yet further aspect of the invention.

Pharmaceutical Formulations and Methods of Administration

Any of the compounds herein described may be administered in the form of a pharmaceutical composition comprising said compound, together with one or more pharmaceutically acceptable carriers, excipients or diluents. Acceptable carriers, excipients and diluents for therapeutic use are well known in the art and can be selected with regard to the intended route of administration and standard pharmaceutical practice. Examples include binders, lubricants, suspending agents, coating agents, solubilising agents, preserving agents, wetting agents, emulsifiers, surfactants, sweeteners, colourants, flavouring agents, odorants, buffers, antioxidants, stabilising agents and/or salts.

The compounds described herein may be formulated with one or more conventional carriers and/or excipients according to techniques well known in the art. For example, these may be formulated in conventional oral administration forms, e.g. tablets, coated tablets, capsules, powders, granulates, solutions, dispersions, suspensions, syrups, emulsions, etc. using conventional excipients, e.g. solvents, diluents, binders, sweeteners, aromas, pH modifiers, viscosity modifiers, antioxidants, etc. Suitable excipients may include, for example, corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose or fatty substances such as hard fats or suitable mixtures thereof, etc.

It is envisaged that the compositions described herein will generally be administered by other conventional administration routes, e.g. topically or parenterally. Where parenteral administration is employed this may, for example, be by means of intravenous, subcutaneous, intramuscular or intraocular injection. Intravenous injection typically requires high dosages of the active to achieve efficacious drug levels within the eye and can be subject to physiological barriers to success due to the need for the active to cross the blood-retina barrier. Intraocular injection (intravitreal) is thus generally preferred. For this purpose, sterile solutions containing the active compound may be employed, such as an oil-in-water emulsion.

The use of topical administration forms, e.g. eye drops, lotions, creams, ointments, irrigants, gels, lenses, foams, sprays, tinctures, or pastes, is especially preferred since these permit delivery of the active compound directly to the eye and thus avoid side effects of systemic administration, e.g. adverse effects on the heart or liver. Such administration forms are especially advantageous due to their ease of administration and low attendant risk of infection (as can be the case with intravitreal injection, for example). Various types of carriers may be used for topical formulations, including both aqueous and non-aqueous carriers.

Any of the topical formulations described herein may further comprise at least one delivery agent that assists in the penetration of at least one surface of the eye. In certain embodiments, the delivery agent may assist in delivery of the active agent to the cornea and/or the retina of the eye. In order for a topical application to be effective in treatment of conditions at the back of the eye, the active compound needs to be able to penetrate the surface of the eye so that it can reach the posterior segment of the eye, i.e. the retina. The penetration rate should be sufficient to impart an effective dose. Pharmaceutically acceptable drug delivery agents include any of the agents disclosed in WO 2013/049621, the entire content of which is incorporated herein by reference. Examples of such agents include lecithin, D-α-tocopherol, polyethylene glycol 1000 succinate, surfactants such as Tweens and other similar polymeric delivery matrices.

Other delivery agents capable of targeting the active agent to the posterior segment of the eye include non-aqueous liquid vehicles comprising perfluorocarbons, semifluorinated alkanes, polysiloxanes or mixtures of these. Examples of such delivery agents are disclosed in U.S. Pat. No. 9,241,900, EP 2444063 and U.S. Pat. No. 5,874,469, the entire contents of which are incorporated herein by reference.

As would be understood, topical administration to the eye generally refers to localised administration to a surface of the eye, e.g. to any exterior surface of the eye normally accessible between the eyelids.

Solutions comprising the compounds described herein are particularly preferred due to the patient's ability to easily administer such compositions by means of instilling one to two drops of the solution into the affected eye(s). However, the compounds for use according to the invention may also be readily incorporated into other types of compositions, such as suspensions, emulsions, viscous or semi-viscous gels, or other types of semi-solid compositions. Suspensions or emulsions, such as oil-in-water emulsions, are preferred. The compositions may also include various other ingredients, such as buffers, preservatives, co-solvents, and/or viscosity enhancing agents. Where water is present, an appropriate buffer system (e.g., sodium phosphate, sodium acetate or sodium borate) may be added to prevent pH drift under storage conditions. Where a viscosity enhancing agent is present this typically enhances the viscosity of the ophthalmic/topical formulation to increase retention time of the solution on the eye. Viscosity enhancing agents include, among others, carbopol gels, dextran 40, dextran 70, gelatine, glycerin, polyoxyethylene-polyoxypropylene block copolymer, carboxymethylcellulose, hydroxyethyl cellulose, hydroxypropyl methylcellulose, polyethylene glycol, polysorbate 80, propylene glycol, polyvinyl alcohol and polyvinlylpyrrolodine (povidone). The desired amount of viscosity enhancing agent for use in the ophthalmic formulations can be determined by one skilled in the art.

Emulsions (e.g. microemulsions) comprising a polar phase (e.g. water), a non-polar phase (e.g. oil), and at least one surfactant represent a preferred delivery vehicle for the active compounds herein described. Oil-in-water emulsions, e.g. oil-in-water microemulsions, are particularly preferred. In microemulsions the oil phase droplets typically have a mean diameter of less than about 300 nm, e.g. between about 5 nm and about 200 nm. Such formulations are considered to be effective even in treating diseases of the posterior segment of the eye. Suitable microemulsions include those described in US 2014/0275263, the entire contents of which are incorporated herein by reference.

An oil-in-water emulsion (e.g. an oil-in-water microemulsion) comprising any of the lipid compounds herein described (e.g. a compound of formula (I) or a compound of formula (II)), or a pharmaceutically acceptable salt thereof, represents a further embodiment of the invention.

Cyclodextrins are useful excipients in eye drop formulations for a variety of lipophilic drugs and may be used in any of the formulations herein desrcibed. They facilitate eye drop formulations for drugs that otherwise might not be available for topical use, while improving absorption and stability and decreasing local irritation.

Lipid nanoparticles may be used as a drug delivery system for the compounds herein described in order to enhance their ocular bioavailability following topical administration. Lipid nanoparticles of mean diameter between about 50 and 400 nm are suitable for ocular administration. Their typical composition comprises physiological and biodegradable/biocompatible lipids which are suitable for the incorporation of lipophilic and hydrophilic drugs within the lipid matrix in large amounts. The matrix is stabilized with one or more surfactants in aqueous dispersion. Suitable lipids for lipid nanoparticle preparation include triglycerides, propylene glycol dicaproylcaprate, diglycerides, monoglycerides, glyceryl palmitostearate, aliphatic alcohols and fatty acids (E. B. Suoto et al, Current Eye Research, 2010, 35 (7), 537-552).

Ophthalmic products are typically packaged in multidose form. Preservatives are thus required to prevent microbial contamination during storage and use. Suitable preservatives include: benzalkonium chloride, thimerosal, chlorobutanol, methyl paraben, propyl paraben, phenylethyl alcohol, edetate disodium, sorbic acid, polyquaternium-1 or other agents known to those skilled in the art. Such preservatives are typically employed at a level of from 0.001 to 1.0% w/v.

The dosage required to achieve the desired activity of the compounds herein described will depend on various factors, such as the compound selected, its mode of administration, whether the treatment is therapeutic or prophylactic, and the nature and severity of the ocular disorder, etc. Typically, a physician will determine the actual dosage which will be most suitable for an individual subject. The specific dose level and frequency of dosage for any particular patient may be varied and will depend upon factors such as the activity of the specific compound employed, the metabolic stability and length of action of that compound, the age of the patient, the mode and time of administration, and the severity of the particular condition. The compound and/or the pharmaceutical composition may be administered in accordance with a regimen of from 1 to 10 times per day, such as once or twice per day. For oral, topical and parenteral administration to human patients, the daily dosage level of the agent may be in single or divided doses.

Suitable daily dosages of the compounds herein described are 0.1 mg to 1 g of said compound; 1 mg to 500 mg of said compound; 5 mg to 100 mg of said compound; 5 mg to 50 mg of said compound, or 10 mg to 50 mg of said compound, or 0.1 mg to 10 mg of said compound per day. By a “daily dosage” is meant the dosage per eye per 24 hours. Where provided in a form for topical administration (e.g. instillation) directly into the eye, a suitable amount of the active compound may be in the range of 0.001 to 95% (w/v) of the composition; a range from 0.001% to 50% (w/v) of the composition; a range from 0.005% to about 40% (w/v); a range from 0.01% to 35% (w/v), a range from 0.05% to 30% (w/v), a range from 0.1% to 25% (w/v); a range from 1% to 20% (w/v), a range from 1% to 10% (w/v), or a range from 1% to 5% (w/v). For example, when the compositions are dosed topically, they will generally be employed in a concentration range of from about 5 to about 10% w/v, with 1-2 drops administered per eye 1-4 times per day. A suitable daily dosage of the active compound for topical administration may be up to 100 mg per eye per day.

Clinical Conditions

The ophthalmic disorder to be treated or prevented by the compounds herein described may be any disease or disorder associated with the eye. It may be a disease of the anterior segment of the eye. Examples of these include cataract, corneal neovascularization, dry eye syndrome, glaucoma, keratitis and keratocornus. Alternatively, it may be a disease of the posterior segment of the eye which includes the vitreous humor, retina, retinal blood vessels, macula, choroid and optic nerve. Of particular interest are inflammatory, autoimmune, vascular and infectious diseases of the eye (e.g. those affecting the posterior segment of the eye). Examples of these include age-related macular degeneration (including dry-form AMD and wet-form AMD), diabetic retinopathy, diabetic macular edema, uveitis, retinitis pigmentosa, Stargardt's disease, retinal inflammation, ocular inflammation, corneal inflammation, retinal vascular leakage and endophthalmitis.

The compounds herein described are particularly suitable for the treatment or prevention of any ocular inflammatory disease (OD). Ocular inflammatory disease is a general term for inflammation affecting any part of the eye or surrounding tissue. Inflammation involving the eye can range from the familiar allergic conjunctivitis associated with hayfever to rare, potentially blinding conditions such as uveitis, scleritis, episcleritis, optic neuritis, keratitis, orbital pseudotumor, retinal vasculitis, and chronic conjunctivitis.

Many retinal degenerative disorders are inherited, meaning that they are due to a genetic mutation. There are many types of inherited retinal degenerations which may be treated according to the invention. Examples of these include retinitis pigmentosa, choroideremia (affects males), Leber congenital amaurosis, retinoschisis (juvenile), Stargardt's disease, Usher disease, and Bardet Biedl disease.

A number of ophthalmologic diseases have increasingly been recognised to result in part from impaired mitochondrial function, increased oxidative stress, and increased apoptosis. For example, primary mitochondrial diseases that are caused by mutations in either the nuclear genome or mitochondrial genome frequently involve clinically significant ophthalmologic disease that most commonly involves the optic nerve, retina, extraocular eye muscles, and eyelids. Amongst these diseases are the following, all of which may be treated or prevented in accordance with the invention:

Primary Inherited Mitochondrial Diseases:

Dominant Optic Atrophy (DOA). DOA is a genetic disease that primarily affects the retinal ganglion cells (RGC) and nerve fiber layer of the retina. The prevalence of DOA is estimated at 1 in 35,000 individuals in northern Europe. Visual acuity typically decreases over the first two decades of life to a mean of 20/80 to 20/120.

Leber Hereditary Optic Neuropathy (LHON). This is characterized by acute and painless central vision loss of both eyes in a sequential fashion over a period of days to months. LHON was the first maternally-inherited ophthalmologic disorder to be linked to a point mutation in mitochondrial DNA. LHON has a recognised disease prevalence estimated at 1 in 25,000 in England and other areas of Europe.

Pigmentary retinopathy and other ophthalmologic problems. Pigmentary retinopathy is a non-specific finding that may be found in several mitochondrial diseases. The best described primary mtDNA disease in which pigmentary retinopathy may be seen is Neurogenic weakness, Ataxia, and Retinitis Pigmentosa (NARP). Pigmentary retinopathy can also occur in a range of other mtDNA cytopathies including Leigh syndrome (a degenerative disorder involving the basal ganglia and brainstem), Mitochondrial Encephalomyopathy Lactic Acidosis and Stroke (MELAS), Myoclonic Epilepsy and Ragged Red Fibers (MERRF), LHON, Kearns-Sayre Syndrome (KSS), and mitochondrial myopathy

As a high energy demand organ, the eye is particularly susceptible to the consequences of mitochondrial damage. Mitochondria are a major site of oxidative stress generation and scavenging. In addition, mitochondria are the mediators of cellular apoptosis that is initiated by the release of cytochrome c from the mitochondrial intermembrane space, where it plays an integral role in energy generation within the respiratory chain. Oxidative damage that results over time from mtDNA instability leads to cumulative mitochondrial damage, which is recognized to be an important pathogenic factor in age-related ophthalmologic disorders such as diabetic retinopathy, age-related macular degeneration, and glaucoma. This understanding has unleashed a range of emerging therapeutic approaches for mitochondrial-based ophthalmologic disorders directed at optimizing mitochondrial function (Schrier and Falk, Curr. Opin. Ophthalmol. September 2011; 22(5): 325-331). In recent years, it has become evident that mitochondrial dysfunction, perhaps through alterations in oxidative stress balance, contribute to a wide range of common and complex ophthalmologic diseases of aging, such as diabetic retinopathy, AMD, and glaucoma. These are described in more detail below:

Age-related macular degeneration (AMD)—retinal degeneration, particularly including AMD, is responsible for a large proportion of blindness in the elderly population. Light appears to have a deleterious effect on retinal cells that already have compromised mitochondrial function. Wavelengths of light ranging from 400 to 760 nm appear to specifically affect tissues that are replete with mitochondria by reducing the activity of mitochondrial dehydrogenases and increasing the release of reactive oxygen species.

Diabetic retinopathy—this is the leading cause of blindness in young adults. The pathogenesis of diabetic retinopathy involves progressive dysfunction of retinal mitochondria in the setting of hyperglycemia, with mtDNA damage and accelerated apoptosis occurring in retinal capillary cells.

Glaucoma—this is the second-leading cause of blindness worldwide. It is an optic neuropathy that manifests with optic nerve cupping and atrophy similar to what is observed in primary mitochondrial optic neuropathies. Increasingly persuasive evidence suggests that glaucomatous tissue damage is initiated by elevated intraocular pressure and/or tissue hypoxia also involves oxidative stress. Experimental elevation of intraocular pressure induces oxidative stress in the retina. It also appears that mitochondrial oxidative stress may have an important role in glaucomatous neurodegeneration.

Uveitis—Intraocular inflammation, commonly referred to as uveitis, is a principal causative factor underlying blindness from retinal photoreceptor degeneration. Oxidative retinal damage in uveitis is caused by activated macrophages, which generate various cytotoxic agents, including inducible nitric oxide produced by inducible nitric oxide synthase, O2. and other reactive oxygen species (ROS).

Cataract—oxidative stress plays a significant role in cataractogenesis. The lens is highly susceptible to ROS, and mitochondria are located in the epithelium and superficial fiber cells. In these cell types, the mitochondria have been confirmed as the major source of ROS generation. Several in vitro studies have demonstrated that human lens cells are highly susceptible to oxidative insults, in which antioxidant activity is generally inversely proportional to cataract severity (see Jarrett et al., Ophthalmic Res. 2010; 44:179-190).

The compounds described herein may also be used to treat any ophthalmic diseases or disorders related to oxidative stress in any compartment of the eye, a dysregulation of the NF-κB signaling pathway or mitochondrial dysregulation and mtDNA damage such as: Stargardt's macular dystrophy, age related macular degeneration, retinal detachment, hemorrhagic retinopathy, retinitis pigmentosa, cone-rod dystrophy, Sorsby's fundus dystrophy, optic neuropathy, inflammatory retinal disease, diabetic retinopathy, diabetic maculopathy, retinal blood vessel occlusion, retinopathy of prematurity, ischemia reperfusion related retinal injury, proliferative vitreoretinopathy, retinal ischemia, retinal dystrophy, hereditary optic neuropathy, uveitis, any retinal injury, blepharitis, nya retinal disorder associated with Alzheimer's disease, any retinal disorder associated with multiple sclerosis, any retinal disorder associated with Parkinson's disease, any retinal disorder associated with a viral infection, any retinal disorder related to light overexposure, myopia, any retinal disorder associated with AIDS, macular edema, cataract, keroconjunctivitis sicca, Stevens-Johnson syndrome, Sjögrens syndrome, post-cataract surgery, dry eye, allergic conjunctivitis, neuropathic ocular pain, posterior capsular opacification and intraocular tumors.

Preparation of Compounds for use in the Invention

Certain compounds of formula (I) are known in the art, or can be prepared by methods known to those skilled in the art. For example, omega-3 lipid compounds and methods for their preparation are described in WO 2010/008299, WO 2008/053331, WO 2010/128401, WO 2008/142482 and WO 2012/059818, the entire contents of which are incorporated herein by reference.

The novel omega-6 lipid compounds of formula (II) may be prepared from readily available starting materials using synthetic methods known in the art, for example, using methods analogous to those described in WO 2010/008299, WO 2008/053331, WO 2010/128401, WO 2008/142482 and WO 2012/059818 (see above). Suitable starting materials include natural omega-6 fatty acids such as linoleic acid (LA) ((all-Z)-9,12-octadecadienoic acid), gamma-linolenic acid (GLA) ((all-Z)-6,9,12-octadecatrienoic acid), calendic acid (8E,10E,12Z-octadecatrienoic acid), eicosadienoic acid ((all-Z)-11,14-eicosadienoic acid), dihomo-gamma-linolenic acid (DGLA) ((all-Z)-8,11,14-eicosatrienoic acid), arachidonic acid (AA) ((all-Z)-5,8,11,14-eicosatetraenoic acid), docosadienoic acid ((all-Z)-13,16-docosadienoic acid), adrenic acid ((all-Z)-7,10,13,16-docosatetraenoic acid), and docosapentaenoic acid ((all-Z)-4,7,10,13,16-docosapentaenoic acid).

Compounds of the general formula (II) where X is —CH₂— may be prepared by Methods 1 to 4 as described below:

Method 1:

This method is suitable for preparing compounds of formula (II) where R³ is hydrogen and R² denotes a C₁-₆ alkyl group, a halogen atom, or an acyl group, and R⁴ is a group of the formula —COOR⁵ where R⁵ is hydrogen or an alkyl group:

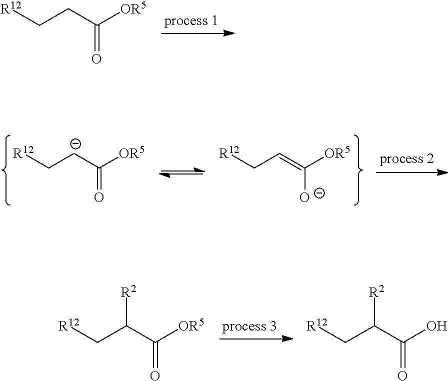

A long chain omega-6 polyunsaturated ester is reacted with a strong non-nucleophilic base (e.g. lithium diisopropylamine, potassium/sodium hexamethyldisilazide or KH/NaH/DMF) in a solvent such as tetrahydrofuran or diethyl ether at temperatures of −60 to −78° C., to provide the ester enolate (process 1). This ester enolate is reacted with an electrophilic reagent such as an alkylhalide (e.g. ethyliodine), an acylhalide (e.g. acetylchloride), a carboxylic anhydride (e.g. acetic anhydride) or an electrophilic halogenation reagent (e.g. N-fluorobenzene sulfonamide (NFSI), N-bromosuccinimide or iodine) to provide a mono-substituted derivative (process 2). The resulting ester is optionally further hydrolysed in a solvent such as methanol or ethanol to produce the carboxylic acid derivative by addition of a base such as lithium/sodium hydroxide in water at temperatures between 15 and 80° C. (process 3).

Method 2:

This method is suitable for preparing compounds of formula II where R³ is hydrogen, R² is hydroxy or an alkoxy group, and R⁴ is a group of the formula —COOR⁵ where R⁵ is hydrogen or an alkyl group:

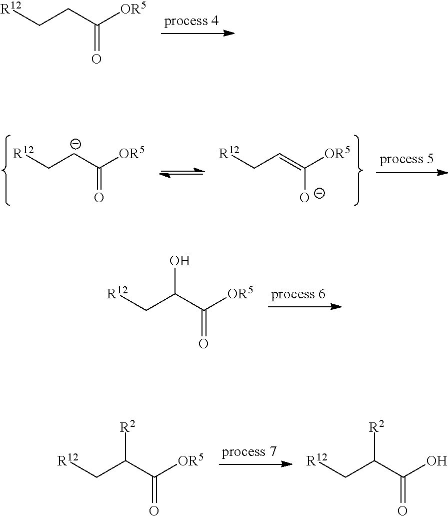

An omega-6 acid ester is reacted with a strong nucleophilic base such as lithium diisopropylamine or potassium/sodium hexamethyldisilazideane in a solvent such as tetrahydrofuran or diethyl ether at a temperature of −60 to −78° C. to provide an ester enolate (process 4). The ester enolate is reacted with an oxygen source such as dimethyldioxirane, 2-(phenylsulfonyl)-3-phenyloxaziridine, or molecular oxygen, optionally with additives such as trimethylphosphite or catalysts such as a Ni(II) complex, to provide an alpha-hydroxy ester (secondary alcohol) (process 5). Reaction of the secondary alcohol with a base such as sodium hydride in a solvent such as THF or DMF generates an alkoxide which is then reacted with an electrophilic reagent such as an alkyliodide (e.g. methyl iodide or ethyl iodide) (process 6). The ester thus produced is optionally hydrolysed in a solvent such as ethanol or methanol to the carboxylic acid derivative by addition of a base such as lithium/sodium hydroxide in water at a temperature between 15 and 80° C. (process 7).

Method 3:

The alpha hydroxyl esters produced in Method 2 above are useful intermediates for the introduction of other functional groups in the α-position. For example, the hydroxyl function can be activated by conversion to a halide or tosylate prior to reaction with different nucleophiles such as ammonia, amines, thiols, etc. The Mitsunobu reaction may also be used to convert the hydroxyl group into other functional groups.

Method 4:

Compounds represented by the general formula (II) where R³ is a hydrogen atom and R² denotes an alkyl, carboxyl, hydroxyl, or alkoxy group can be prepared by reacting a long chain polyunsaturated tosylate, mesylate or halide with a substituted dialkylmalonate. Hydrolysis and decarboxylation gives the desired alpha-substituted products.

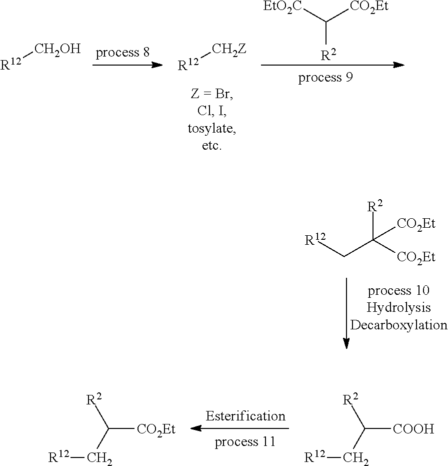

The long chain polyunsaturated tosylates used in Method 4 can be prepared from the corresponding long chain omega-6 polyunsaturated alcohol.

Compounds of general formula (II) where X is oxygen may be prepared by Methods 5 to 7 as detailed below wherein “LG” denotes a leaving group, suitably a halogen, or mesylate or tosylate group:

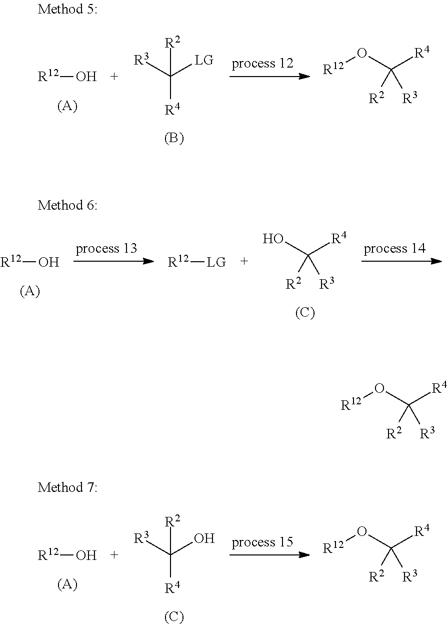

Alcohols of formula (A) used in Methods 5, 6 and 7 may be prepared directly from the carboxylic esters of, for example, naturally occurring omega-6 fatty acids by reduction with a reducing agent such as lithium aluminum hydride (LAH) or diisobutyl aluminum hydride (DIBAL-H) at -10° C. to 0° C. The alcohol can also be prepared by reduction of (all-Z)-pentadeca-3,6,9-trienal made by degradation of arachidonic acid as described, for example, by Corey et al. (Tetrahedron Letters, 1983, Vol. 24, 265-268). The alcohols of formula (A) may be prepared starting from a purified omega-6 fatty acid, but it is also possible to start with a natural fatty acid mixture containing the omega-6 fatty acid. Such mixtures can come from different algae. The omega-6 alcohols can also be made by standard synthetic methods such as acetylene chemistry followed by selective hydrogenation. Several such methods are described by S. Durand et al. (J. Chem. Soc., Perkin Trans. 1, 2000, 253-273). Viala et al. (J. Org. Chem, 1988, 53, 6121) have also developed several syntheses for PUFAs using either a 3-carbon or a 6-carbon homologating agent to build up the polyene system with Wittig reactions.

Compounds of formulae (B) and (C) can be prepared by standard processes known in the art. The leaving group (LG) present in the compounds of formula (B) may, for example, be mesylate, tosylate or a suitable halogen, such as bromine, chlorine or iodine. Other suitable leaving groups will be apparent to the skilled person.

In Method 5 the alcohols of formula (A) are reacted in a substitution reaction with a compound of formula (B) in the presence of a base such as an alkali metal hydroxide, for example NaOH in an appropriate solvent system. Suitable solvent systems include a two-phase mixture of toluene and water.

In Method 6 the alcohols of formula (A) can be converted using functional group interconversion using methods familiar to a person skilled in the art to produce compounds where the terminal hydroxy group has been transformed into a suitable leaving group (process 13). Suitable leaving groups include bromine, mesylate, and tosylate, or others that will be apparent to the skilled person. These compounds can be reacted further (process 14) in a substitution reaction with the appropriately substituted hydroxy acetic acid derivatives (compounds of formula (C)), in the presence of a base in an appropriate solvent system.

In Method 7 an alcohol of formula (A) can be reacted with an appropriately substituted hydroxy acetic acid derivative (compound of formula (C)), under classic or non-classic Mitsunobu conditions, using methods familiar to persons skilled in the art.

Compounds of general formula (II) where X is sulfur may be prepared by Method 8 or Method 9 as described below wherein “LG” denotes a leaving group, suitably a halogen, or mesylate or tosylate group:

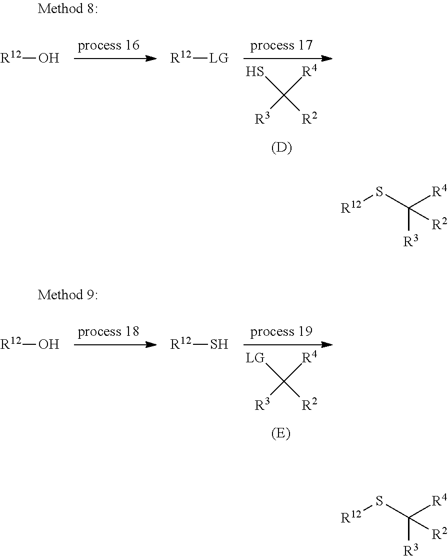

Compounds (D) and (E) are commercially available, or they are known in the literature, or they are prepared by standard processes known in the art. The leaving group (LG) present in the compounds of formula (E) may, for example, be mesylate, tosylate or a suitable halogen, such as bromine.

In Method 8 the starting alcohols R¹²—OH can be converted, using functional group interconversion, by methods familiar to persons skilled in the art (process 16), to produce compounds where the terminal hydroxyl group has been transformed into a suitable leaving group (LG). Suitable leaving groups include bromine, mesylate and tosylate. These compounds can be reacted further (process 17) in a substitution reaction with the appropriate substituted thiol acetic acid derivative (D), in the presence of base.

Using Method 9, the alcohols R¹²—OH can be converted to the corresponding thiols by methods familiar to persons skilled in the art (process 18). The thiols can then be reacted further (process 19) in a substitution reaction with compounds of formula (E) in the presence of base in an appropriate solvent system.

If the acid derivatives prepared in any of the methods herein described are carboxylic esters, hydrolysis can be performed to obtain the free fatty acids. An esterifying group such as a methyl or an ethyl group may be removed, for example, by alkaline hydrolysis using a base such as an alkali metal hydroxide, for example LiOH, NaOH or KOH, or by using an organic base, for example Et₃N together with an inorganic salt, for example LiCl in an appropriate solvent system. A tert-butyl group may be removed, for example, by treatment with an acid, for example an organic acid such as trifluoroacetic acid or formic acid in an appropriate solvent system. Suitable solvent systems may comprise dichloromethane.

Conversion of a compound of formula (II) in the form of a carboxylic acid to a corresponding salt can be performed by suitable methods known in the art, for example, by treating it with a suitable base in an appropriate solvent system. Removal of the solvent will give the resulting salt.

As will be understood, the preparation of compounds of formula (II) according to Methods 1 to 8 may in some cases result in mixtures of stereoisomers. If required, these isomers may be separated by means of chiral resolving agents and/or by chiral column chromatography using methods known to the person skilled in the art.

As described herein, the omega-6 lipid compounds of formula (II) can also be provided in the form of derivatives, e.g. as phospholipids, mono-, di- or tri-glycerides. Such derivatives can be prepared according to methods known in the art. Suitable methods include those described in Methods 10 to 13 below:

Method 10:

The compounds of formula (II) wherein R⁴ is a carboxylic acid derivative in the form of a phospholipid can be prepared via this process. Acylation of sn-glycero-3-phosphocholine (GPC) with an activated fatty acid, such as fatty acid imidazolides, is a standard procedure in phosphatidylcholine synthesis. It is usually carried out in the presence of DMSO anion with DMSO as a solvent (see e.g. Hermetter, Chemistry and Physics of lipids, (1981) 28, 111).

Sn-Glycero-3-phosphocholine as a cadmium (II) adduct can also be reacted with the imidazolide activated fatty acid in the presence of DBU (1,8-diazabicyclo[5.4.0]undec-7-ene) to prepare the phosphatidylcholine of the respective fatty acid (see, for example, PCT/GB2003/002582). Enzymatic transphosphatidylation can effect the transformation of phosphatidylcholine to phosphatidyletanolamine (see e.g. Wang et al, J. Am. Chem. Soc, (1993) 115, 10487).

Phospholipids may also be prepared by enzymatic esterification and transesterification of phospholipids or enzymatic transphosphatidylation of phospholipids (see e.g. Hosokawa, J. Am. Oil Chem. Soc. 1995, 1287, and Lilja-Hallberg, Biocatalysis, (1994) 195).

Method 11:

The compounds of formula (II) wherein R⁴ is a carboxylic acid derivative in the form of a triglyceride can be prepared through the following process. Excess of the fatty acid can be coupled to glycerol using dimethylaminopyridine (DMAP) and 2-(1H-benzotriazol-1-yl)-N,N,N′,N′-tetramethyluroniumhexafluorophosphate (HBTU).

Method 12:

The compounds of formula (II) wherein R⁴ is a carboxylic acid derivative in the form of a diglyceride can be prepared by reaction of the fatty acid (2 equivalents) with glycerol (1 equivalent) in the presence of 1,3-dicyclohexylcarbondiimide (DCC) and 4-dimethylaminopyridine (DMAP).

Method 13:

The compounds of formula (II) wherein R⁴ is a carboxylic acid derivative in the form of a monoglyceride can be prepared through the following process. Acylation of 1,2-0-isopropylidene-sn-glycerol with a fatty acid using DCC and DMAP in chloroform gives a monodienoylglycerol. Deprotection of the isopropylidene group can be effected by treating the protected glycerol with an acid (e.g. HCl, acetic acid, etc.) (see e.g. O'Brian, J. Org. Chem., (1996) 5914).

There are several synthetic methods for the preparation of monoglycerides with the fatty acid in the 2-position. One method involves esterification of the fatty acid with glycidol in the presence of 1-(3-dimethylaminopropyl)-3-ethylcarbodiimidehydrochloride (EDC) and 4-dimethylaminopyridine (DMAP) to produce a glycidyl derivative. Treatment of the glycidyl derivative with trifluoroacetic anhydride (TFAA) prior to trans-esterification gives rise to the monoglyceride (see e.g. Parkkari et al, Bioorg. Med. Chem. Lett. (2006) 2437).

It is also possible to use enzymatic processes (lipase reactions) for the transformation of a fatty acid to a mono-, di-, tri-glyceride. A 1,3-regiospecific lipase from the fungus Mucor miehei can, for example, be used to produce triglycerides or diglycerides from polyunsaturated fatty acids and glycerol. The non-regiospecific yeast lipase from Candida antartica is also highly efficient in generating triglycerides from polyunsaturated fatty acids. (see e.g. Haraldsson, Pharmazie, (2000), 3).

The invention will now be described in more detail by way of the following non-limiting Examples and with reference to the accompanying figures, in which:

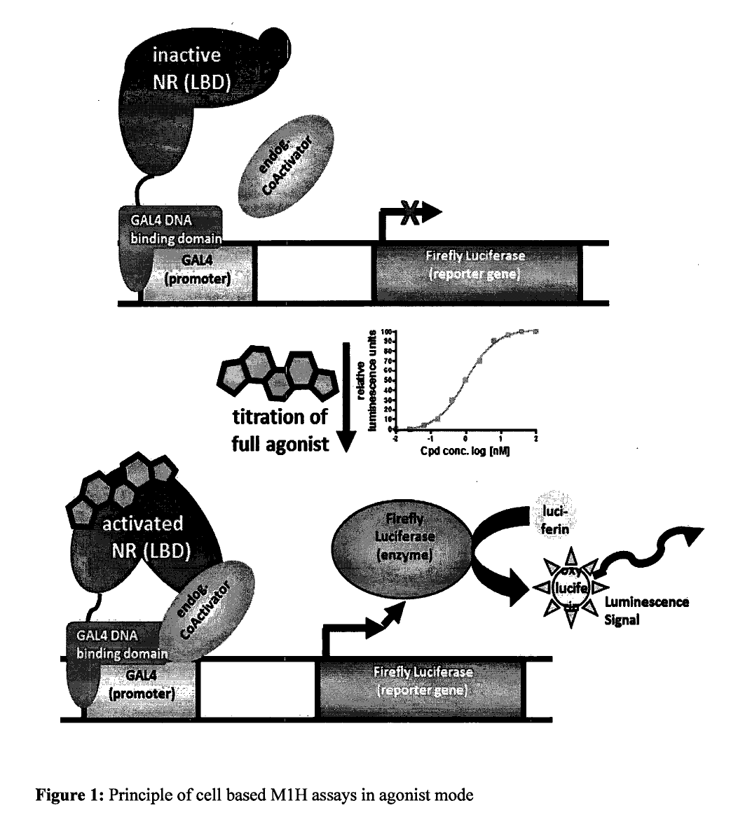

FIG. 1—illustrates the principle of cell based M1H assays in agonist mode used in Example 6;

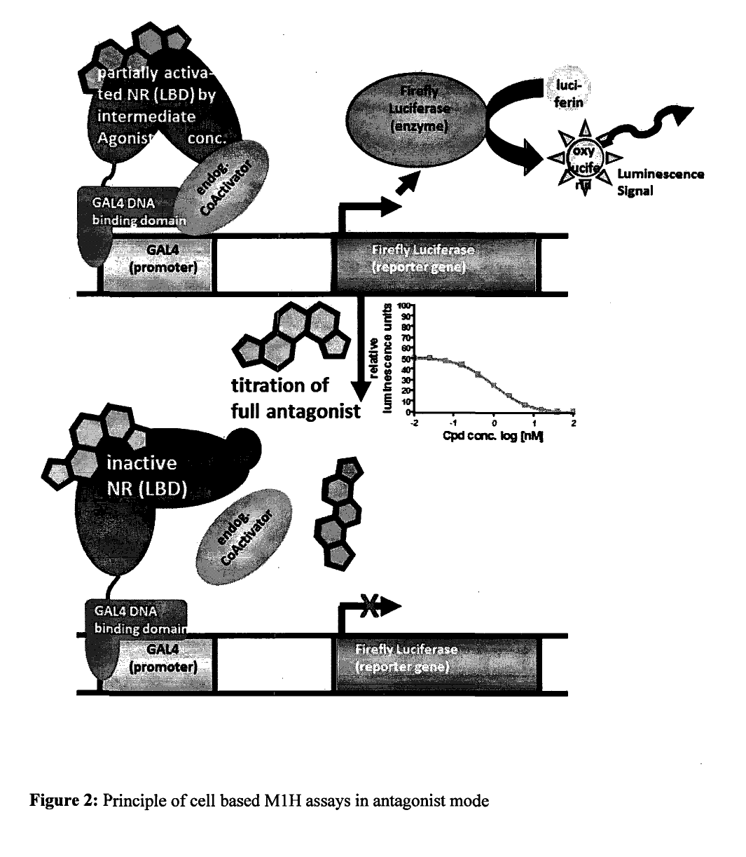

FIG. 2—illustrates the principle of cell based M1H assays in antagonist mode used in Example 6;

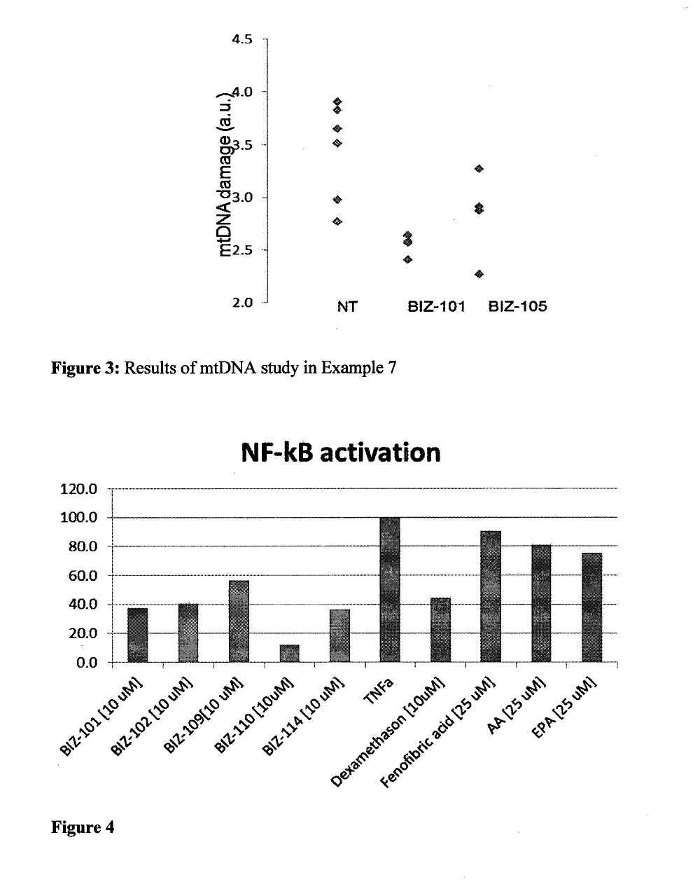

FIG. 3—shows the results of the mitochondrial DNA study in Example 7;

FIG. 4—shows the effects of compounds on the activity of the NF-κB pathway using the NF-κB reporter assay in Example 22;

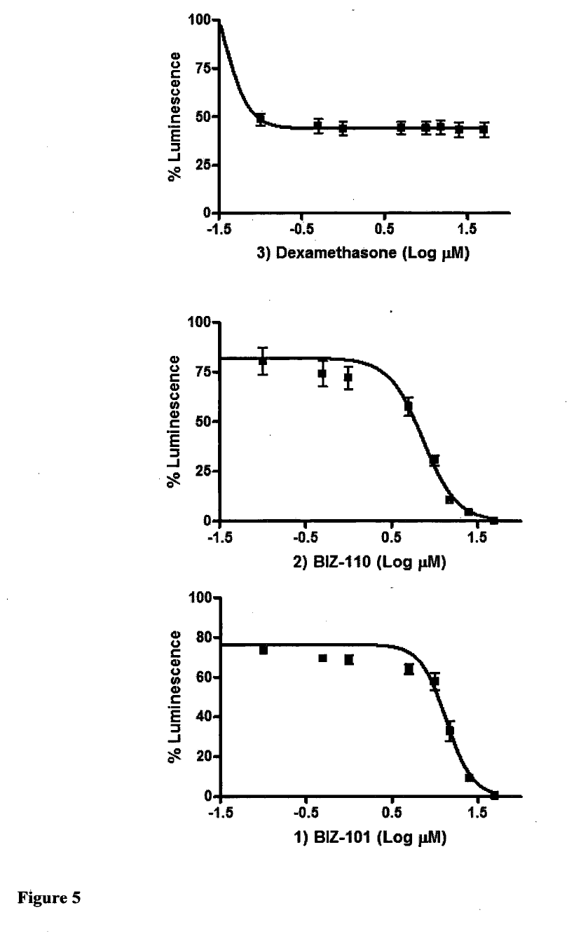

FIG. 5—shows the dose dependence of the inhibitory effect of dexamethasone, compound BIZ-110 and compound BIZ-101 on the NF-κB pathway;

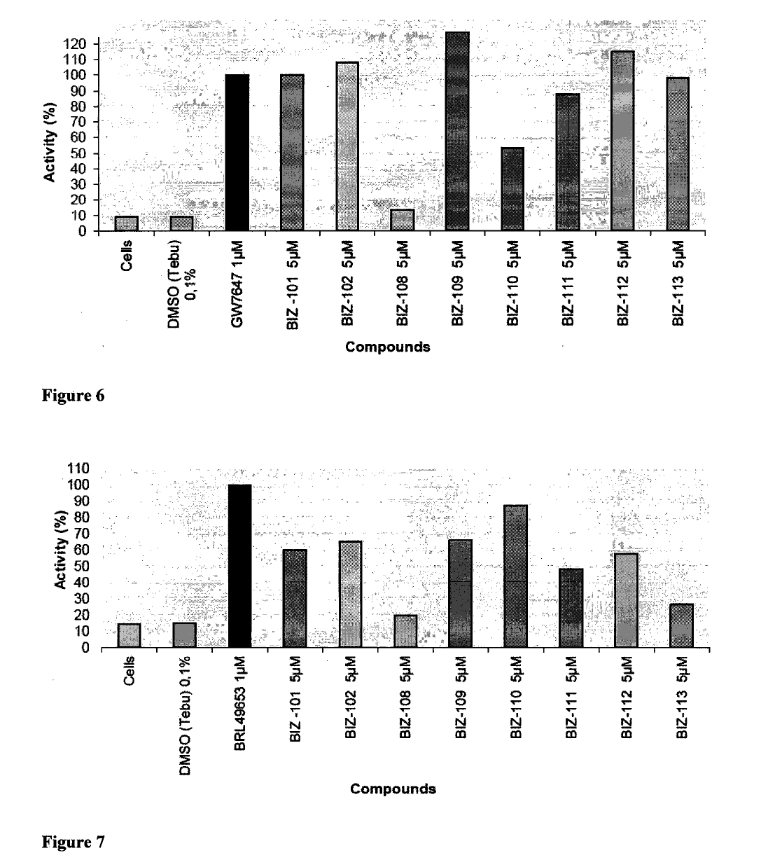

FIG. 6—shows the PPARα activation of compounds at 5 μM concentration;

FIG. 7—shows the PPARγ activation of compounds at 5 μM concentration;

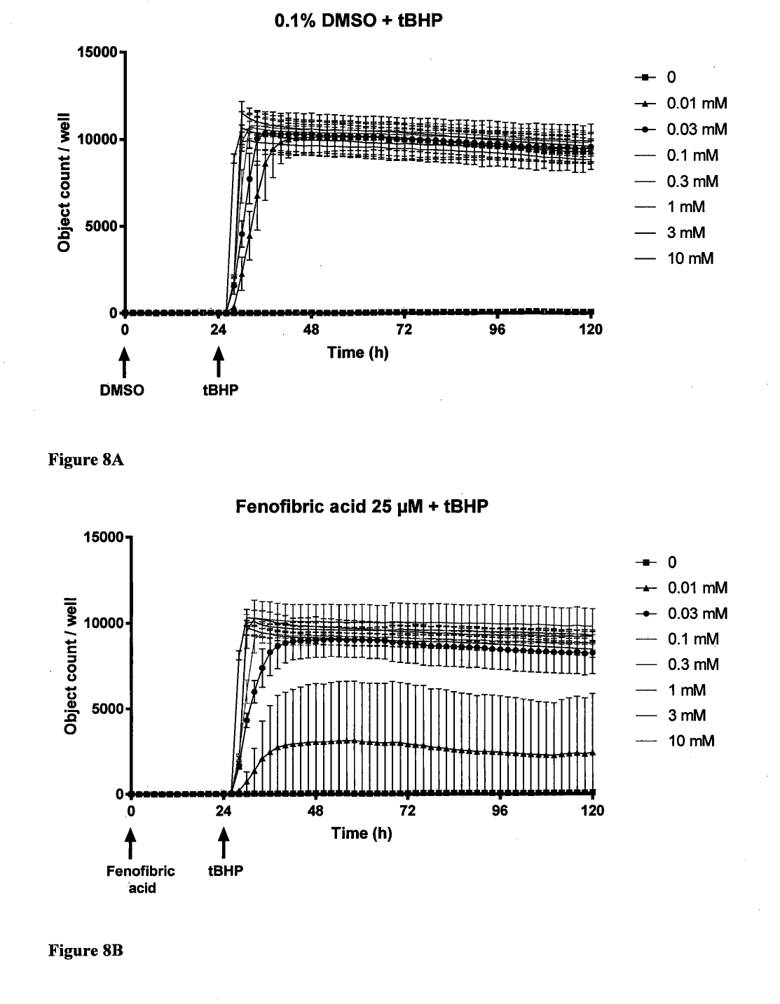

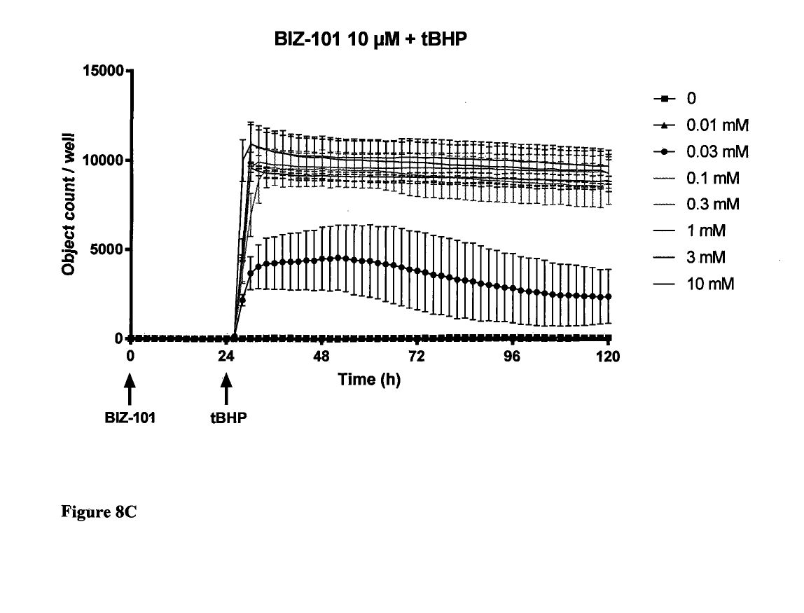

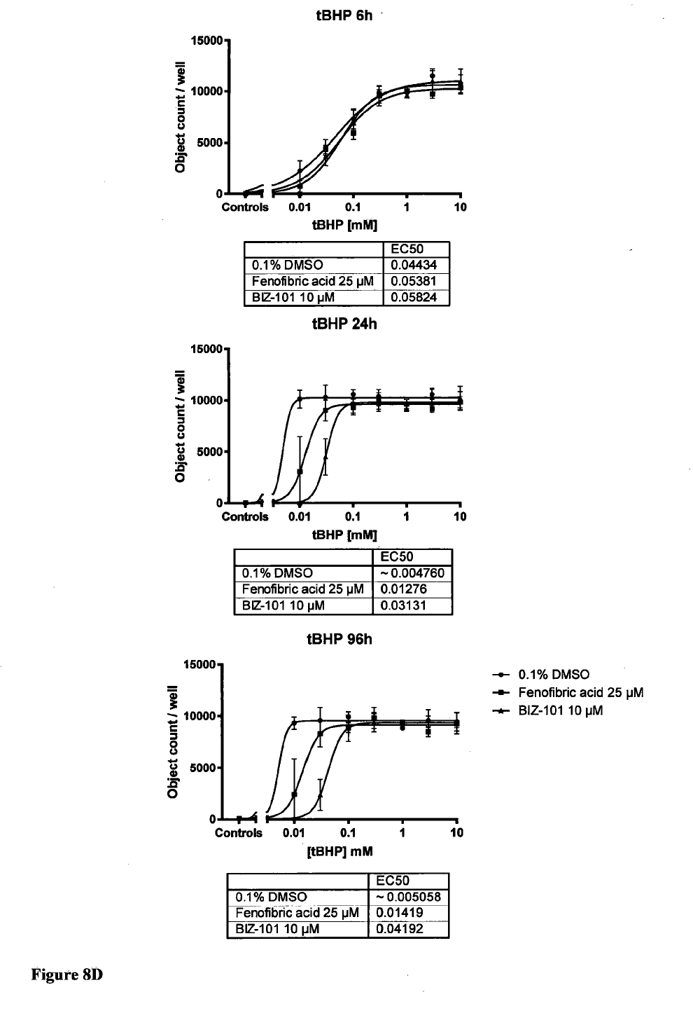

FIG. 8—shows kinetic monitoring of cytolysis in ARPE-19 cells treated with 0.1% DMSO (8A), Fenofibric acid (8B), or BIZ-101 (8C). FIG. 8D shows the dose response analysis of the tBHP cytotoxicity after 6, 24 and 96 hours; and

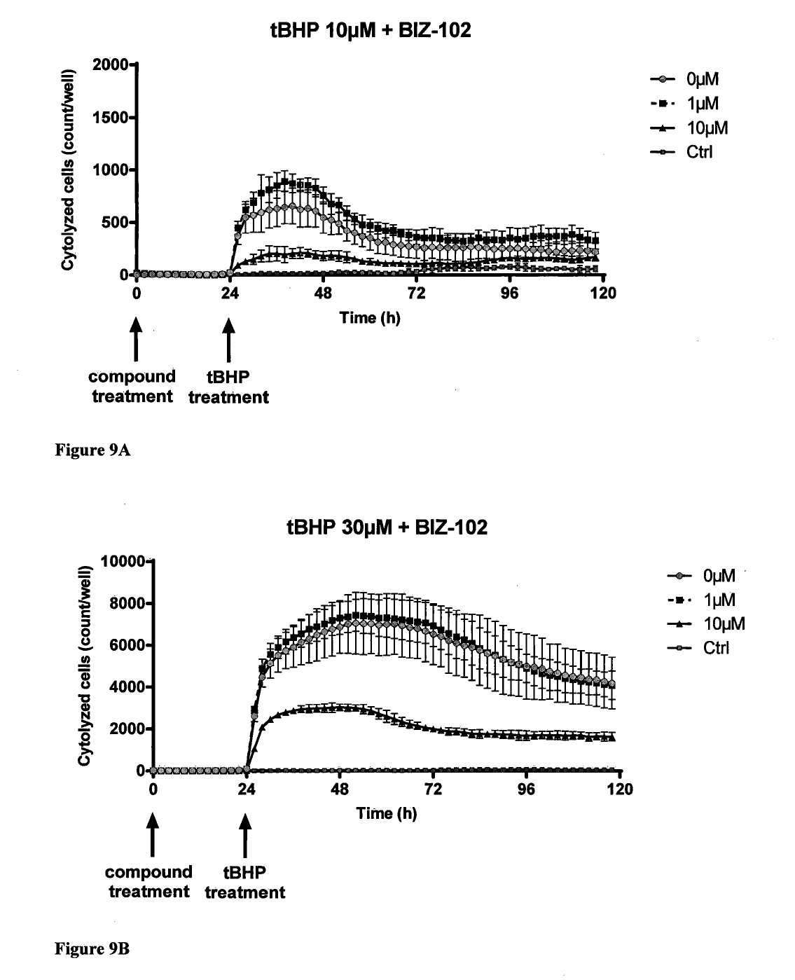

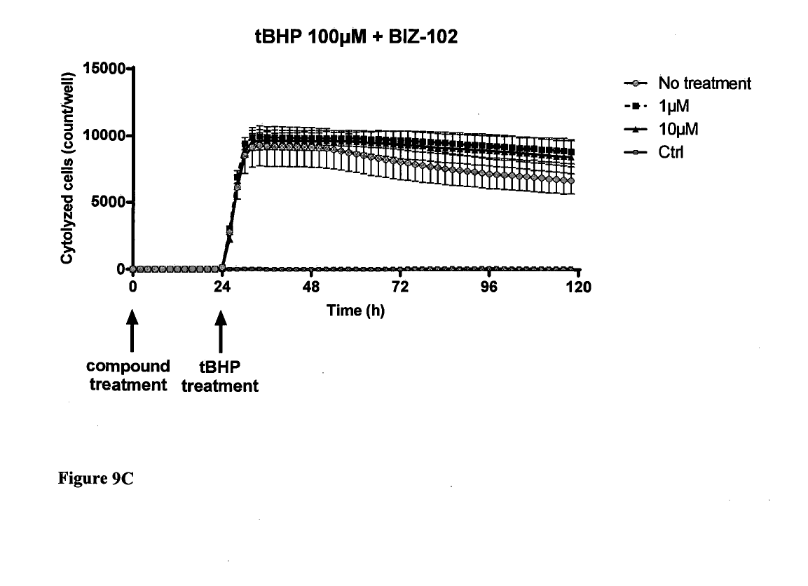

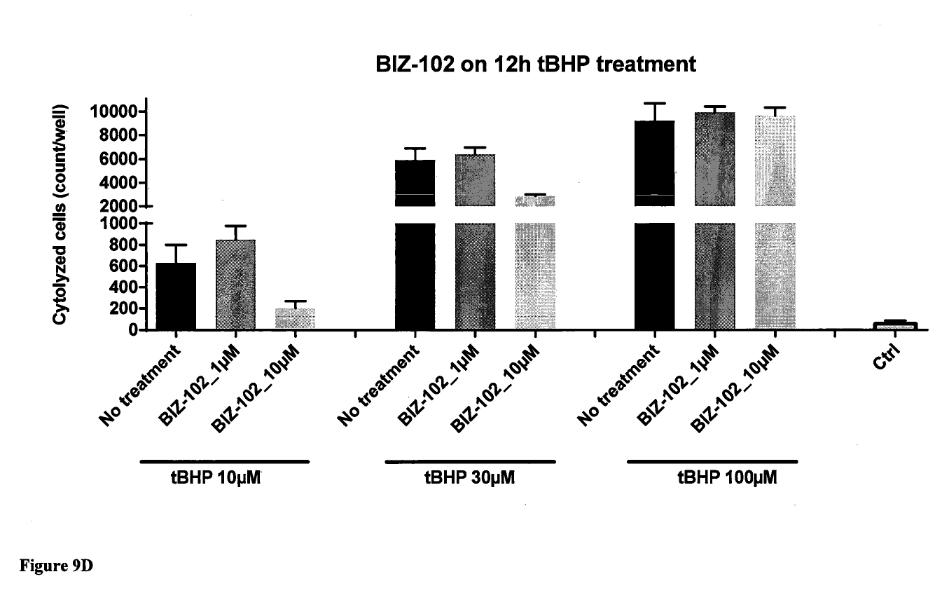

FIG. 9—shows kinetic monitoring of cytolysis in ARPE-19 cells treated with compound BIZ-102. Cytotoxicity is induced by 10 μM t-BHP (9A), 30 μM t-BHP (9B), and 100 μM t-BHP (9C). In FIG. 9D the effects of BIZ-102 12 hrs after addition of tBHP is shown.

The NMR-spectra were recorded in CDCl₃, with a Bruker Avance DPX 200 or DPX 300 or DPX 400 instrument. Mass spectra were recorded at 70 eV with a Fision VG Pro spectrometer. All reactions were performed under nitrogen or argon atmosphere.

### EXAMPLE 1

### Preparation of ethyl (all-Z) 2-ethyl-5,8,11,14-eicosatetraenoate

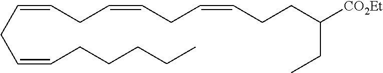 [SMILES: CCCCC/C=C\C/C=C\C/C=C\C/C=C\CCC(CC)C(=O)OCC]

Butyl lithium (0.96 ml, 1.54 mmol in 1.6 M in hexane) was added dropwise to a stirred solution of diisopropylamine (0.23 ml, 1.6 mmol) in dry THF (5 ml) under a nitrogen atmosphere at 0° C. The resulting solution was stirred at 0° C. for 20 minutes, cooled to −78° C. and stirred an additional 10 minutes before dropwise addition of ethyl (all-Z)-5,8,11,14-eicosatetraenoate (466 mg, 1.4 mmol) in dry THF (5 ml). The mixture was stirred at −78° C. for 10 minutes before addition of ethyl iodide (170 μl, 2.09 mmol). The mixture was allowed to warm to room temperature over 1 hour. The mixture was then poured into water, and extracted with heptane. The combined organic phase was washed with 1M HCl and then dried (Na₂SO₄). Filtration and evaporation under reduced pressure followed by flash chromatography (2% EtOAc in hexane) gave compound (1) as a clear oil (450 mg, 89% yield).

### EXAMPLE 2

### Preparation of (all-Z) 2-ethyl-5, 8,11,14-eicosatetraenoic acid (BIZ 102)

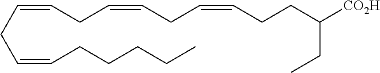 [SMILES: CCCCC/C=C\C/C=C\C/C=C\C/C=C\CCC(CC)C(=O)O]

Ethyl (all-Z) 2-ethyl-5,8,12,15-eicosatetraenoate produced in Example 1 (450 mg, 1.25 mmol) was dissolved in 30 ml ethanol and a solution of LiOH (420 mg) in water (10 ml) was added. The mixture was left stirring at 80° C. under an argon atmosphere for 18 hours. The mixture was cooled, then a 1M HCl solution (15 ml) was added and the mixture extracted with ether. The organic phase was washed with brine and dried (MgSO₄). Filtration and evaporation gave the acid as a light yellow oil (416 mg) in 100% yield.

δ_{H} (400 MHz, CDCl₃) δ0.89 (t, 3H , J=7.0,), 0.95 (t, 3H, J=7.4,), 1.16-1.45 (m, 6H), 1.45-1.83 (m, 4 H), 1.87-2.20 (m, 4H), 2.34 (tt, 1H, J=8.4, 5.5), 2.62-2.96 (m, 6H) 5.56-5.13 (m, 8H), δ_{C} (101 MHz, CDCl₃) 11.83, 14.23, 22.74, 25.21, 25.31, 25.77, 25.80, 27.38, 29.49, 31.68, 46.37, 127.72, 128.05, 128.32, 128.37, 128.72, 128.86, 129.17, 130.66, 180.83

### EXAMPLE 3

### Preparation of 2-ethyleicosa-(all-Z)-5,8,11,14,17-pentaenoic acid (α-ethyl EPA) (BIZ-101)

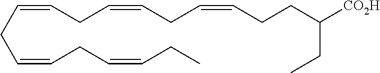 [SMILES: CC/C=C\C/C=C\C/C=C\C/C=C\C/C=C\CCC(CC)C(=O)O]

BIZ-101 was prepared based on the procedure described by Larsen et al., Biochemical Pharmacology, 1998, 405.

Butyl lithium (2.25 ml, 3.6 mmol in 1.6 M in hexane) was added dropwise to a stirred solution of diisopropylamine (594 μl, 4.2 mmol) in dry THF (5 ml) under a nitrogen atmosphere at −20° C. The resulting solution was stirred at −78° C. for 45 min before dropwise addition of ethyl (all-Z)-5,8,11,14,17-eicosapentaenoate (1.0g, 3.0 mmol) in dry THF (20 ml). The mixture was stirred at −78° C. for 30 minutes before addition of ethyl iodide (388 μl, 4.8 mmol). The mixture was stirred at 0° C. for 30 min before being poured into water (5 ml). The water phase was separated and extracted with hexane (210 ml). The combined organic phase was washed with 2M HCl (5 ml), water (25 ml) and then dried (MgSO₄). Filtration and evaporation under reduced pressure followed by flash chromatography (2% EtOAc in hexane) gave ethyl (all-Z)-2-ethyl-5,8,11,14,17-eicosapentaenoate as a clear oil (670 mg, 63% yield).

Ethyl (all-Z)-2-ethyl-5,8,11,14,17-eicosapentaenoate (670 mg, 1.9 mmol) was dissolved in a mixture of ethanol/THF (15 ml, 1:1) and a solution of LiOH (550 mg) in water (7.5 ml) was added. The mixture was left stirring at room temperature for 18 hours. Water and hexane were added and the organic phase was collected. The water phase was acidified with 5% HCl to pH 2 and extracted three times with hexane:ethylacetate (7:3). The organic phase was washed with water and brine and dried (MgSO₄). Filtration and evaporation gave the acid as a light yellow oil (429 mg) in 68% yield.

δ_{H} (200 MHz) δ0.93 (t, 3H, J=7.3), 0.96 (t, 3H, J=7.5), 1.39-1.85 (m, 4H), 1.95-2.19 (m, 4H), 2.22-2.42 (m, 1H), 2.68-2,95 (m, 8H), 5.21-5.52 (m, 10H), δ_{C}(50 MHz): δ12.4, 15.0, 21.2, 25.6, 25.7, 26.1, 26.2, 31.9, 46.8, 126.4, 127.2, 127.5, 127.6, 127.9, 128.0, 128.4, 131.3, 181.6

### EXAMPLE 4

### Preparation of 2-methyldocosa-(all-Z)-4,7,10,13,16,19-hexaenoic acid (α-methyl DHA) (BIZ-105)

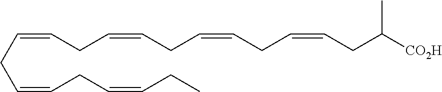 [SMILES: CC/C=C\C/C=C\C/C=C\C/C=C\C/C=C\C/C=C\CC(C)C(=O)O]

BIZ-105 was prepared based on the procedure described by Larsen et al., Lipids, Vol. 40, 2005.

Butyl lithium (1.12 ml, 1.7 mmol in 1.5 M in hexane) was added dropwise to a stirred solution of diisopropylamine (283 μl, 2.0 mmol) in dry THF (4.2 ml) under nitrogen atmosphere at −20° C. The resulting solution was stirred at −78° C. for 45 min before dropwise addition of ethyl (all-Z)-4,7,10,13,16,19-docosahexaenoate (500 mg, 1.4 mmol) in dry THF (8.4 ml). The mixture was stirred at −78° C. for 30 minutes before addition of methyl iodide (140 μl, 4.8 mmol). The mixture was stirred at 0° C. for 30 min before being poured into water (5 ml). The water phase was separated and extracted with hexane (210 ml). The combined organic phase was washed with 2M HCl (5 ml), water (25 ml) and then dried (MgSO₄). Filtration and evaporation under reduced pressure gave ethyl (all-Z)-2-methyl-4,7,10,13,16,19-docosahexaenoate as a clear oil (520 mg, 100% yield).

Ethyl (all-Z)-2-methyl-4,7,10,13,16,19-docosahexaenoate (520 mg, 1.4 mmol) was dissolved in a mixture of ethanol/THF (9 ml, 2:1) and a solution of LiOH (437 mg) in water (6 ml) was added. The mixture was left stirring at room temperature for 18 hours. Water and hexane were added and the organic phase was collected. The water phase was acidified with 5% HCl to pH 2 and extracted three times with hexane:ethylacetate (7:3). The organic phase was washed with water and brine and dried (MgSO₄). Filtration and evaporation gave the acid as a light yellow oil (390 mg) in 81% yield. δ_{H} (300 MHz): 0.96 (t, 3H, J=7.5 Hz), 1.18 (d, 3H, J=6.8 Hz), 2.06 (m, 2H), 2.20-2.30 (m, 1H), 2.35-2.55 (m, 2H), 2.75-2.95 (m, 10 H), 5.25-5.55 (m, 12 H); τ_{C} (75 MHz) 14.25, 16.34, 20.55, 25.53, 26.63, 30.90, 39.41, 126.32, 127.01, 127.87, 127.98, 128.08, 128.11, 128.23, 128.56, 130.26, 132.03, 128.27, 182.37; m/z (CI) 343 (M+1, 1.65%), 215, 93, (100), HRMS: found M+1 343.262563.

### EXAMPLE 5

### Preparation of 2-methyl-tetradecylthioacetic acid (a-methyl TTA) (BIZ-103)

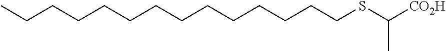 [SMILES: CCCCCCCCCCCCCCSC(C)C(=O)O]

BIZ-103 was prepared based on the procedure described in EP-A-0345038 and US 2004/0192908.

Potassium hydroxide (34.30 g, 0.611 mol), 2-mercapto propionic acid (31.2 g, 0.294 mol) and 1-bromotetradecane (50 ml, 0.184 mol) were added in that order to methanol (400 ml) and stirred overnight at room temperature. A concentrated hydrochloric acid solution (60 ml) dissolved in water (800 ml) was then added to the reaction mixture. Precipitation of 2-methyl-tetradecylthioacetic acid occurred. The mixture was stirred overnight at room temperature. The precipitate was then filtered, washed five times with water and dried. The product was recrystallized from methanol and isolated as white flakes by filtration (yield 90%). TLC gave only one spot with iodine vapor.

δ_{H} (400 MHz, CDCl₃): 0.89 (t, 3H, J=6.8 Hz), 1.2-1.3 (m, 20 H), 1.3-1.4 (m, 2 H), 1.46 (d, 3H, J=7.2 Hz), 1.5-1.7 (m, 2H), 2.6-2.7 (m, 2H), 3.41 (q, 1 H, J=7.2 Hz); δ_{C} (101 MHz, CDCl₃): 14.13, 16.90, 22.70, 28.88, 29.19, 29.21, 29.37, 29.50, 29.60, 29.66, 29.68, 29.70, 31.67, 31.93, 40.88, 179.27

### EXAMPLE 6

### PPARα Activity of Compounds

The compounds BIZ-102 (Example 2), BIZ-103 (Example 5) and BIZ-106 were tested at the human PPARalpha receptor in a cellular GAL4 Reporter gene assay. The assay was run in agonist and antagonist mode to detect agonistic as well as antagonistic activities of the tested compounds.

Materials and Methods:

Compounds Tested:

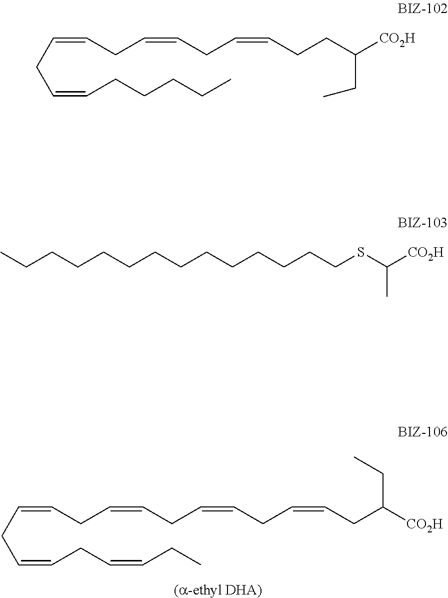 [SMILES: CCCCC/C=C\C/C=C\C/C=C\C/C=C\CCC(CC)C(=O)O ; CCCCCCCCCCCCCCSC(C)C(=O)O ; CC/C=C\C/C=C\C/C=C\C/C=C\C/C=C\C/C=C\CC(CC)C(=O)O]

Reference Compounds:

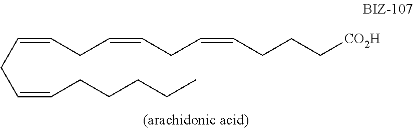 [SMILES: CCCCC/C=C\C/C=C\C/C=C\C/C=C\CCCC(=O)O]

GW7647 (a known PPARα agonist)

Fenofibric acid (a known PPARα agonist)

GAL4 Transactivation Assays:

FIG. 1 shows the principle of the cell based M1H assays in agonist mode. FIG. 2 shows the principle of the cell based M1H assays in antagonist mode.

Performance of the GAL4 cellular reporter assay:

Day 1: Cells were seeded in 96 well plates in plating medium (MEM with serum), incubation overnight at 37° C., 5% CO₂.

Day 2: Removal of plating medium

Addition of PEI-based transfection agent

Incubation for 4-6 hours at 37° C., 5% CO₂

Addition of assay medium (MEM with serum)

Addition of compounds (dilution series was generated in MEM with serum)

Incubation overnight at 37° C., 5% CO₂

Day 3: Removal of assay medium (16-20 hours after compound addition)

Addition of Passive Lysis Buffer (Promega)

15 mins incubation at room temperature

Addition of luciferase buffers and measurement in a dual-flash procedure

The assays were done in HEK293 cells (DSMZ ACC 305). The plasmids used in the GAL4 assay system were derivatives of Stratagene's M2H plasmids: the reporter plasmid pFR-Luc (containing a synthetic promoter with five tandem repeats of the yeast GAL4 binding sites that control expression of the Photinus pyralis (American firefly) luciferase gene), and pCMV-BD (for fusions of nuclear receptor ligand binding domains to the DNA-binding domain of the yeast protein GAL4). In order to improve experimental accuracy, a second reporter—Renilla reniformis luciferase, driven by a constitutive promoter—was included as an internal control. Using the control reporter (Renilla Luciferase) allowed corrections for variations in experimental handling, e.g. transfection efficacy, cell viability, pipetting errors, cell lysis efficiency and assay efficiency. For the antagonist mode experiments, the medium added after transfection contained intermediate concentrations of the reference compound GW7647 (2.5 nM).

Data Evaluation:

Primary read out of the assays was loaded into PhAST (Phenex Assay and Screening Tool) and checked for assay quality (generation of SB and Zprime values). These data were then loaded into the Analysis tool of PhAST to generate graphs and dose response curves. Within PhAST there are two measured and one calculated data layers:

LAYER1 (measured)—contains the activity values of the firefly luciferase activities and is a direct measure for modulation of the cofactor binding properties of the Nuclear Receptors by the tested compounds.

LAYER2 (measured)—contains the activity values of the renilla luciferase activities and is used as normalisation layer. As the renilla luciferase is expressed under control of the constitutively active CMV promoter, moderate well-to-well differences can be used to correct for variations in experimental handling.

LAYER3 (calculated)—is calculated according to the following equation:

1000 * Firefly luciferase value/Renilla luciferase value

These normalised values are, as well as LAYER1, a measure for the modulation of the cofactor binding properties of the nuclear receptors by the tested compounds.

Results and Discussion:

Results were obtained for the 10 compound concentration triplicate assays in direct Firefly and Renilla normalized measurement mode. Dose response curves were used to determine the EC₅₀ values for BIZ-102 and BIZ-103 as 350 nM and 154 nM. Compared with the reference compound GW7647 the tested compounds showed an efficacy of ˜150 and ˜180%. Compared with fenofibric acid, the efficacies of the tested compounds are ˜45 and ˜55%, respectively.

The EC₅₀ values for BIZ-106 and BIZ-107 were determined from dose response curves to be ˜5 μM and ˜26 μM, respectively. Compared with the reference compound GW7647 the tested compounds show an efficacy of ˜80 and ˜100%, respectively. Compared with fenofibric acid, the efficacies of the tested compounds are ˜25 and ˜30%, respectively.

All tested compounds showed agonistic effects at PPARα in antagonist mode. For evaluation of agonistic effects it is better to use agonist mode data as they are only influenced by the test compound. The table below shows the obtained potencies and efficacies in detail.

| Compound | | | Efficacy vs. | Efficacy vs. |
|---|---|---|---|---|
| tested | EC₅₀ value | | GW7647 | Fenofibric Acid |
| BIZ-102 | 350 | nM | 150% | 45% |
| BIZ-103 | 154 | nM | 180% | 55% |
| BIZ-106 | 5 | μM | 80% | 25% |
| BIZ-107 | 26 | μM | 100% | 30% |

It can be seen that BIZ-102 and BIZ-103 activated PPARα at nanomolar concentrations and their efficacy was significantly better than the reference compound GW7647. Although their efficacy is not as high as fenofibric acid, this compound is not active on PPARα at nanomolar concentrations (EC₅₀ fenofibric acid measured as 10-18 μM).

### EXAMPLE 7

### PPARβ, γ and δ Activity of Compounds

The compounds BIZ-102 and BIZ-103 were tested at the human PPARbeta/delta and PPARgamma receptors using the same cellular GAL4 Reporter gene assays as in Example 6. The assays were run in agonist mode to detect agonistic activities of the tested compounds. Both tested compounds showed weak agonistic effects at PPARbeta/delta and PPARgamma.

The table below shows the obtained potencies and efficacies in detail.

| | PPARbeta/delta | | PPARgamma | |
|---|---|---|---|---|
| | | efficacy vs. | | efficacy vs. |
| | EC₅₀ value | GW501516 | EC₅₀ value | Rosiglitazone |
| BIZ-102 | 4.3 μM | 20% | 1.5 μM | 23% |
| BIZ-103 | 8.9 μM | 18% | 1.6 μM | 20% |

### EXAMPLE 8

### Effect on Mitochondrial DNA (mtDNA)

Mitochondrial DNA damage is a useful biomarker to evaluate the potential therapeutic effect of the compounds in relation to the treatment of retinal degenerative diseases.

Method:

In this study, neuroblastoma cells were cultivated at near confluency (50-75%) in DMEM/F12/10% serum high glucose (20 mM) supplied with 10 μM BIZ-101 or BIZ-105 for 24 hours prior to analyses. These conditions readily induce mtDNA damage that is representative of that accumulating during aging as the result of age-associated oxidative stress.

For DNA damage analyses, cells were washed, and DNA isolated using Qiagen Blood&Tissue DNA isolation kit. DNA damage level was analyzed by a RT-qPCR method based on the ability of DNA lesions to inhibit restriction enzyme cleavage, as described previously (http://www.ncbi.nlm.nih.gov/pubmed/25631007) using primers 5′-aaactgctcgccagaacact-3′ and 5′-catgggctacaccttgacct-3′ (sense and antisense, respectively). Briefly, genomic DNA (6 ng) was treated with 1 U TaqI for 15 min at 65° C. DNA damage frequency was calculated as 2exp-(ctTaqI-ctnt), where ctTaqI and ctnt represent CT values of TaqI-treated and non-treated genomic DNA, respectively.

Results:

The results from the experiment demonstrated reduced mtDNA damage in in vitro cultured neuroblastoma cells upon BIZ-101 administration and a slightly reduced level of mtDNA damage by BIZ-105 (see FIG. 3; NT=no treatment). The results indicate at least the potential for BIZ-101 to be used for treatment of retinal degenerative diseases like AMD.

### EXAMPLE 9

### Preparation of 2-ethyl (4Z,7Z,10Z,13Z,16Z,19Z)-docosa-4,11,10,13,16,19-hexaenoic acid (BIZ 106)

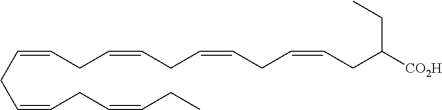 [SMILES: CC/C=C\C/C=C\C/C=C\C/C=C\C/C=C\C/C=C\CC(CC)C(=O)O]

Butyl lithium (0.96 ml, 1.54 mmol in 1.6 M in hexane) was added dropwise to a stirred solution of diisopropylamine (0.23 ml, 1.6 mmol) in dry THF (5 ml) under a nitrogen atmosphere at 0° C. The resulting solution was stirred at 0° C. for 20 minutes, cooled to −78° C. and stirred an additional 10 minutes before dropwise addition of ethyl ethyl (4Z,7Z,10Z,13Z,16Z,19Z)-docosa-4,11,10,13,16,19-hexaenoate (499 mg, 1.4 mmol) in dry THF (5 ml). The mixture was stirred at −78° C. for 10 minutes before addition of ethyl iodide (0.16 ml, 2.09 mmol). The mixture was allowed to warm to room temperature over 1 hour. The mixture was then poured into water, and extracted with heptane. The combined organic phase was washed with 1M HCl and then dried (Na₂SO₄). Filtration and evaporation under reduced pressure followed by filtration through a silica plug (hexane:EtOAC 98:2) gave the ethyl ester (330 mg):

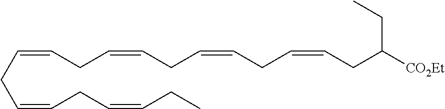 [SMILES: CC/C=C\C/C=C\C/C=C\C/C=C\C/C=C\C/C=C\CC(CC)C(=O)OCC]

The ester (330 mg, 0.9 mmol) was dissolved in 20 ml ethanol and a solution of LiOH (320 mg) in water (10 ml) was added. The mixture was left stirring at 80° C. under an argon atmosphere for 18 hours. The mixture was cooled, then a 1M HCl solution (15 ml) was added and the mixture extracted with ether and dried (MgSO₄). Filtration, evaporation followed by flash chromatography on silica gel (98:2 to 9:1 hexaene/ethylacetate) gave the acid as a light yellow oil (200 mg).

δ_{H} (400 MHz): 0.97 (m, J=7.4, 6H), 1.55-1.75(m, 2H), 2.06 (m, 2H), 2.25-2.50 (m, 2H), 2.75-2.95 (m, 10H), 5.25-5.50 (m, 12 H);

δ_{C} (100 MHz) 11.9, 14.4, 20.7, 24.8, 25.7, 25.79, 25.80, 29.5, 47.1, 126.6, 127.2, 128.0, 128.2, 128.25, 128.29, 128.38, 128.41, 128.42, 128.7, 130.2, 132.2, 181.1

### EXAMPLE 10

### Preparation of (5Z,8Z,11Z,14Z)-icosa-5,8,11,14-tetraenol

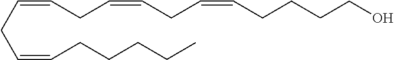 [SMILES: CCCCC/C=C\C/C=C\C/C=C\C/C=C\CCCCO]

A solution of arachidonic acid ethyl ester (3.0 g, 9,0 mmol) in MTBE (15 ml) was added dropwise to a suspension of lithium aluminium hydride (0.68 g, 18 mmol) in MTBE (6 ml) at 0° C. The solution was stirred for an additional 1 hour at 0° C. Water and HCl (2M) were added and the mixture was extracted with diethylether. The combined ether extract was washed with brine and dried (MgSO₄). Evaporation under reduced pressure followed by dry flash on silica gel (50:50 diethyleter/EtOAC) gave the pure alcohol (2.9 g) as an oil.

δ_{H} (400 MHz): 0.86 (t, 3H, J=6.9 Hz), 1.2-1.5 (m, 9H), 1.5-1.6 (m, 2H), 1.95-2.15 (m, 2H), 2.75-2.85 (m, 6H), 3.62 (t, 2 H, J=6.4), 5.2-5.5 (m, 8 H);

δ_{C} (100 MHz) 14.0 (2C), 22.5, 25.6 (2C), 25.7, 26.9, 27.2, 29.3, 31.5, 32.3, 62.8, 127.5, 127.9, 128.0 (2C), 128.3, 128.5, 129.8, 130.4

### EXAMPLE 11

### Preparation of 2-((5Z,8Z,11Z,14Z)-icosa-5,8,11,14-tetraen-1-yloxy) butanoic acid (BIZ 114)

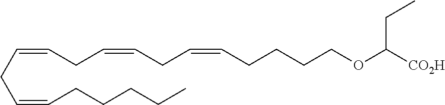 [SMILES: CCCCC/C=C\C/C=C\C/C=C\C/C=C\CCCCOC(CC)C(=O)O]

An aqueous solution of sodium hydroxide (50%, w/w, 3.5 ml) was added to a stirred solution of tetrabutylammonium bromide (133 mg, 0.41 mmol), t-butyl 2-bromobutyrate (930 mg, 4.1 mmol) and (5Z,8Z,11Z,14Z)-icosa-5,8,11,14-tetraenol (566 mg, 1.8 mmol) in toluene (5 ml) at room temperature. The resulting mixture was heated to 30-40° C. and stirred for 3 hours. After cooling to room temperature, a saturated ammonium chloride (NH₄Cl) solution was added and the organic phase was separated. The aqueous phase was extracted with hexane (325 ml). The combined organic layers were washed with NH₄Cl solution, brine and dried (MgSO₄). Filtration and evaporation under reduced pressure followed by flash chromatography on silica gel (98:2 to 95:5 hexaene/ethylacetate) gave the t-butylester as a light yellow oil:

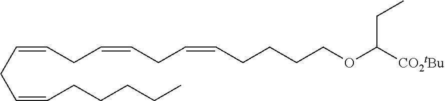 [SMILES: CCCCC/C=C\C/C=C\C/C=C\C/C=C\CCCCOC(CC)C(=O)OC(C)(C)C]

Tert-butyl 2-((5Z,8Z,11Z,14Z)-icosa-5,8,11,14-tetraen-1-yloxy)butanoate was dissolved in formic acid (95%, 6 ml) and stirred at room temperature under nitrogen atmosphere for 2.5 hours. The mixture was concentrated under vacuum and the residue purified by flash chromatography on silica gel (95:5 to 9:1 hexane/EtOAc containing 1% formic acid). Evaporation under reduced pressure gave the fatty acid (87 mg, 9%) as a light yellow oil.

δ_{H} (400 MHz): 0.85 (t, 3H, J=6.9 Hz), 0.97 (t, 3H, J=7.4), 1.2-1.35 (m, 6H), 1.35-1.5 (m, 2H), 1.55-1.65 (m, 2H), 1.7-1.8 (m, 2H), 2.0-2.1 (m, 4H), 2.75-2.85 (m, 6H), 2.3-2.4 (m, 1H), 3.55-3.65 (m, 1H), 3.75-3.85 (m, 1H), 5.2-5.5 (m, 8H) δ_{C} (100 MHz) 9.5, 14.1, 22.6, 25.5, 25.7, 25.9, 26.9, 27.1, 29.2, 29.3, 31.5, 79.5, 127.4, 127.8, 128.0 (2C), 128.2, 128.4, 129.7, 130.4, 178.1.

### EXAMPLE 12

### Preparation of (3Z,6Z,9Z)-pentadeca-3,6,9-tetrien-1-ol

The alcohol was prepared from an algea oil containing arachidonic acid (40%) from Huatai Biopharm

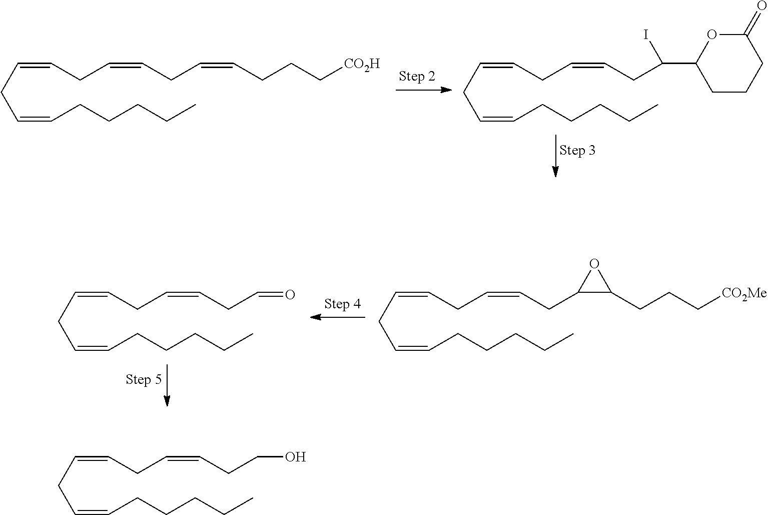 [SMILES: CCCCC/C=C\C/C=C\C/C=C\C/C=C\CCCC(=O)O ; CCCCC/C=C\C/C=C\C/C=C\CC(I)C1CCCC(=O)O1 ; CCCCC/C=C\C/C=C\C/C=C\CC=O ; CCCCC/C=C\C/C=C\C/C=C\CC1OC1CCCC(=O)OC ; CCCCC/C=C\C/C=C\C/C=C\CCO]

Step 1: Hydrolysis of Algea Oil

Algea oil (20 g) dissolved in ethanol (100 ml) was added to a stirred solution of NaOH (16 g) in water (100 ml). The mixture was heated to 60° C. for 2 hrs and left stirring overnight at room temperature. Acetone (200 ml) was added to the mixture and the resulting slurry was filtrated. The filter cake was washed with acetone (2150 ml) and the filtrate evaporated under reduced pressure. Acidification with HCl solution (5%) and extraction with a mixture of hexane:EtOAc (1:1) gave a crude arachidonic acid product (10.5 g) as an oil. The oil was dissolved in hexane and filtered through a silica plug. Evaporation of solvents under reduced pressure yielded the arachidonic acid in a mixture (approx. 40%) of a light yellow oil (5.7 g) which was used without further purification.

Step 2: Iodolactonisation

The crude arachidonic acid produced in step 1 was dissolved in ethanol (35 ml, 80%) and added to a saturated aqueous solution of NaHCO₃ (15 ml). An ethanoic solution (80 ml, 95%) of iodine (2.59 g) was added dropwise under vigorous stirring within an hour. Additional iodine (2g) was added after 1.5 hrs reaction time. The mixture was left stirring overnight and Na₂S₂O₃ was added until the solution was colorless. The mixture was extracted with hexane (3100 ml) and the combined organic layer washed with water, brine and dried (MgSO₄). Filtration and evaporation gave the crude iodolactone (5g).

Step 3: Epoxidation

The crude iodolactone produced in step 3 was dissolved in methanol (100 ml), added to K₂CO₃ (2.7 g) and stirred for 4 hrs. Water was added to the mixture. The mixture was extracted with diethylether and the combined organic layers were washed with a saturated NH₄Cl solution, dried (MgSO₄), filtrated and evaporated under reduced pressure to give the crude epoxide (4 g).

Step 4: Cleavage of epoxide [Procedure from Holmeide & Skattebøl, Journal Chemical Society, Perkin Transactions 1, 2000, 2271]

A solution of crude epoxide (2.6 g) in formic acid (50 ml) and acetic anhydride (5 ml) was stirred at room temperature overnight. Volatile compounds were evaporated under reduced pressure, the residue was dissolved in methanol (65 ml) and K₂CO₃ (1.6 g) was added. After stirring for 3 hrs at ambient temperature water was added and the product extracted with ether. The extract was washed with water and the ether evaporated. The residue was dissolved in methanol (60 ml), cooled to 0° C. and a solution of sodium periodate (2.6 g) in water (20 ml) was added. The mixture was stirred for 1.5 hrs, diluted with water and the product extracted with hexane. The extract was washed with water, dried (MgSO₄) and the solvents evaporated under reduced pressure giving the crude aldehyde (1.9 g).

Step 5: Reduction of Aldehyde

An ice-cooled solution of crude aldehyde (1.9 g) in methanol (100 ml) was added to a solution of NaBH₄ (1.1 g) in methanol (30 ml). The reaction was stirred for 30 minutes before extraction with hexane (3150 ml). The extract was washed with a saturated aqueous NH₄Cl solution and water. The extract was filtered through a plug of silica using a mixture of hexane:EtOAc (95:5). The filtrate was concentrated under reduced pressure to give the alcohol (910 mg) as a colorless oil.

δ_{H} (400 MHz): 0.86 (t, J=6.9, 3H), 1.25-1.35 (m, 6H), 1.40 (s, 1H), 2.03 (q, J=6.8, 2H), 2.30-2.38 (m, 2H), 2.76-2.86 (m, 4H), 6.36 (bt, J=6.3, 2H), 5.23-5.43 (m, 5H), 5.49-5.57 (m, 1H)

δ_{C} (100 MHz): 14.1, 22.6, 25.6, 25.7, 27.2, 29.3, 30.8, 31.5, 62.2, 125.6, 127.5, 127.7, 128.7, 130.5, 131.2.

### EXAMPLE 13

### Preparation of 2-((3Z,6Z,9Z)-pentadeca-3,6,9-tetrien-1-yloxy) butanoic acid (BIZ 111)

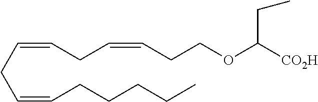 [SMILES: CCCCC/C=C\C/C=C\C/C=C\CCOC(CC)C(=O)O]

An aqueous solution of sodium hydroxide (50%, w/w, 2 ml) was added to a stirred solution of tetrabutylammonium bromide (131 mg, 0.41 mmol), t-butyl 2-bromobutyrate (923 mg, 4.1 mmol) and (3Z,6Z,9Z)-pentadeca-3,6,9-trienol as prepared in Example 12 (400 mg, 1.8 mmol) in toluene (5 ml) at 30° C. The resulting mixture was stirred for 2 hours at 30° C. After cooling to room temperature, a saturated ammonium chloride (NH₄Cl) solution was added and the organic phase was separated. The aqueous phase was extracted with hexane (3×25 ml). The combined organic layers were washed with NH₄Cl solution, brine and dried (MgSO₄). Filtration and evaporation under reduced pressure followed by flash chromatography on silica gel (98:2 to 9:1 hexaene/ethylacetate) afforded the ester (160 mg) containing small amounts of t-butyl-2-bromobutyrate and a pure ester (200 mg) in addition to recovery of the (3Z,6Z,9Z)-pentadeca-3,6,9-trienol (100 mg):

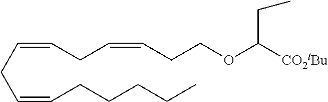 [SMILES: CCCCC/C=C\C/C=C\C/C=C\CCOC(CC)C(=O)OC(C)(C)C]

The pure fraction of the ester (200 mg) was dissolved in formic acid (95%, 3.0 mL). The reaction mixture was left stirring at room temperature for 2 hours. The mixture was concentrated under vacuum, dissolved in hexane (60 ml) and extracted with a saturated NaCO₃ solution. The aqueous phase was acidified using HCl solution and extracted with EtOAc (3×25 ml). The combined organic layers were washed with brine and dried (MgSO₄). Filtration and evaporation under reduced pressure followed by filtration through a short plug of silica (9:1 Hexane/EtOAc+1% formic acid) afforded the title compound (130 mg) as a light yellow oil.

δ_{H} (400 MHz): 0.86 (t, 3H, J=6.9 Hz), 0.98 (t, 3H, J=7.4), 1.20-1.40 (m, 6H), 1.70-1.90 (m, 2H), 2.03 (q, J=6.8, 2H), 2.35-2.45 (m, 2H), 2.75-2.85 (m, 4H), 3.45-3.50 (m, 1H), 3.55-3.65 (m, 1H), 3.83 (dd, J=6.7, J=4.9) 5.2-5.5 (m, 6 H)

δ_{C} (100 MHz) 9.3, 14.0, 22.5, 25.6, 25.3, 25.7, 27.2, 27.9, 29.3, 31.5, 70.3, 77.3, 125.4, 127.4, 127.6, 128.7, 130.50, 130.51, 176.9

### EXAMPLE 14

### Preparation of (2E,6Z,9Z,12Z)-Pentadeca-2,6,9,12-tetraen-1-ol

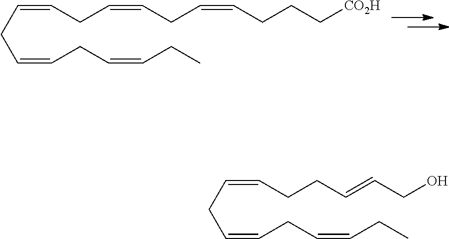 [SMILES: CC/C=C\C/C=C\C/C=C\C/C=C\C/C=C\CCCC(=O)O ; CC/C=C\C/C=C\C/C=C\CC/C=C/CO]

(2E,6Z,9Z,12Z)-Penradeca-2,6,9,12-tetraen-1-ol was prepared from eicosapentaenoic acid as described in the literature (see Flock et al., Acta Chemica Scandinavica, 1999, 53, 436 -Compound 24).

δ_{H} (400 MHz): 0.95 (t, J=7.5, 3H), 1.3 (bs,1H), 2.0-2.2 (m, 6H), 2.7-2.9 (m, 4H), 4.06 (bs, 2H), 5.20-5.45 (m, 6H), 5.6-5.7 (m, 2H)

δ_{C} (100 MHz): 14.2, 20.5, 25.5, 25.6, 26.8, 32.1, 63.7, 127.0, 128.0, 128.35, 128.41, 129.1, 129.4, 132.0, 132.5

### EXAMPLE 15

### Preparation of 2-((2E,6Z,9Z,12Z)-pentadeca-2,6,9,12-tetraen-1-yloxy) butanoic acid (BIZ 112)

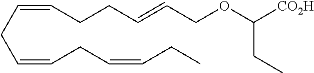 [SMILES: CC/C=C\C/C=C\C/C=C\CC/C=C/COC(CC)C(=O)O]

To a stirred solution of (2E,6Z,9Z,12Z)-Pentadeca-2,6,9,12-tetraen-1-ol (240 mg, 1.1 mmol), t-butyl-2-bromobutyrate (585 mg, 2.6 mmol) and tetrabutylammoniumbromide (71 mg, 0.22 mmol) in toluene (2.5 mL) at room temperature was added an aqueous solution of NaOH (1 ml, 50% w/w). The mixture was stirred at room temperature for 2 hours before addition of a saturated NH₄Cl solution. The organic phase was separated and the aqueous phase was extracted with hexane (3×25 ml). The combined organic phases were washed with brine water and dried (MgSO₄). Filtration and evaporation under reduced pressure followed by flash chromatography on silica (hexane/EtOAc, 95:5) afforded the ester (190 mg):

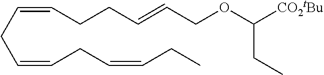 [SMILES: CC/C=C\C/C=C\C/C=C\CC/C=C/COC(CC)C(=O)OC(C)(C)C]

The ester (190 mg) was dissolved in formic acid (95%, 3.0 mL). The reaction mixture was left stirring at room temperature for 2 hours. The mixture was concentrated under vacuum and the residue purified by flash chromatography on silica gel (short coloumn) (95:5 to 9:1 hexane/EtOAc +1% formic acid) to give the title compound (160 mg) as a light yellow oil.

δ_{H} (400 MHz) 0.92-1.0 (dt, J=7.5 J=7.6, 6H), 1.70-1.85 (m, 2H), 2.0-2.2 (m, 6H), 2.75-2.85 (m, 4H), 3.85-3.95 (m, 2H), 4.08 (dd, J=6.1 J=11.7, 1H), 5.2-5.4 (m, 6H), 5.50-5.60 (m, 1H), 5.65-5.75 (m,1H)

δ_{C} (100 MHz) 9.4, 14.2, 20.5, 25.5, 25.6,25.7, 26.6, 32.2, 71.3, 78.2, 125.7, 127.0, 127.9, 128.4, 128.4, 129.0, 132.0, 135.3, 177.57

### EXAMPLE 16

### Preparation of of 2-((5Z,8Z,11Z,14Z,17Z)-icosa-5,8,11,14,17-pentaen-1-yloxy) butanoic acid [corresponding to Example 1 in WO 2010/128401] (BIZ 110)

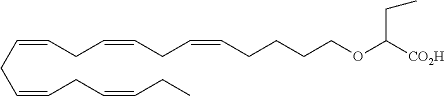 [SMILES: CC/C=C\C/C=C\C/C=C\C/C=C\C/C=C\CCCCOC(CC)C(=O)O]

To a stirred solution of (5Z,7Z,11Z,14Z,17Z)-icosa-5,7,11,14,17-icosa-1-ol (500 mg, 1.7 mmol), t-butyl-2-bromobutyrate (758 mg, 3.4 mmol) and tetrabutylammoniumbromide (110 mg, 0.34 mmol) in toluene (5 mL) at 30° C. was added an aqueous solution of NaOH (1.7 ml, 50% w/w). The mixture was stirred at 40-45° C. for 2.5 hours before additional amounts of t-butyl bromobutyrate (800 mg, 3.5 mmol) and NaOH (0.8 ml, 50% w/w) were added. The mixture was stirred for an additional 1.5 hours at 40-45° C. and cooled to room temperature before addition of a saturated NH₄Cl solution. The organic phase was separated and the aqueous phase was extracted with hexane (3×25 ml). The combined organic phases were washed with NH₄Cl, brine water and dried (MgSO₄). Filtration and evaporation under reduced pressure followed by flash chromatography on silica (hexane/EtOAc, 97:3) afforded the ester (310 mg) containing small amounts of t-butyl-2-bromobutyrate and a pure ester (124 mg):

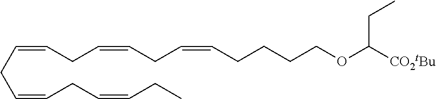 [SMILES: CC/C=C\C/C=C\C/C=C\C/C=C\C/C=C\CCCCOC(CC)C(=O)OC(C)(C)C]

δ_{H} (400 MHz) 0.91-0.98 (dt, J=7.4 J=7.5, 6H), 1.35-1.5 (m, 11 H), 1.55-1.75 (m, 4H), 2.0-2.1 (m, 4H), 2.75-2.85 (m, 8H), 3.25-3.35 (dt, J=6.6 and J=6.6, 1H), 3.55-3.65 (m, 2H), 3.75-3.85 (m, 1H) 5.2-5. 4 (m, 10H)

δ_{C} (100 MHz) 9.7, 14.3, 20.53, 25.51, 25.6, 26.2, 27.0, 28.1, 29.4, 70.2, 80.8, 81.0, 127.0, 127.86, 127.87, 127.9, 128.1, 128.2, 128.45, 128.52, 130.1, 132.0, 172.3

The tert-butylester (310 mg) was dissolved in formic acid (95%, 5.0 mL). The reaction mixture was left stirring at room temperature for 4 hours. The mixture was concentrated under vacuum and the residue purified by flash chromatography on silica gel (95:5 to 0:100 hexane/EtOAc) to give the title compound (120 mg) as a light yellow oil.

δ_{H} (400 MHz) 0.92-0.98 (dt, J=7.4 J=7.5, 6H), 1.35-1.50 (m, 2 H), 1.55-1.70 (m, 2H), 1.70-1.90 (m, 2H), 2.0-2.15 (m, 4H), 2.75-2.85 (m, 8H), 3.41-3.48 (m, 1H), 3.54-3.62 (m, 2H), 3.82 (bt, 1H) 5.2-5. 4 (m, 10H)

δ_{C} (100 MHz) 9.2, 14.3, 20.5, 25.4, 25.5, 25.6, 26.0, 26.9, 29.3, 70.8, 79.7, 127.0, 127.9, 128.05, 128.10, 128.19, 128.23, 128.3, 128.6, 129.7, 132.0, 177.6

### EXAMPLE 17

### Preparation of 2-((4Z,7Z,10Z,13Z,16Z,19Z)-docosa-4,7,10,13,16,19-hexaen-1-yloxy) butanoic acid [corresponding to Example 15 in WO 2010/128401] (BIZ 115)

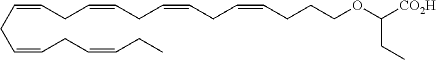 [SMILES: CC/C=C\C/C=C\C/C=C\C/C=C\C/C=C\C/C=C\CCCOC(CC)C(=O)O]

To a stirred solution of (4Z,7Z,10Z,13Z,16Z,19Z)-docosa-4,7,10,13,16,19-hexaen-1-ol (835 mg, 2.6 mmol) in toluene (7 mL) at room temperature, was added tetrabutylammonium bromide (193 mg, 0.6 mmol, 0.23 equiv.) and t-butyl-2-brombutyrate (1.34 g, 6.0 mmol, 2.3 equiv.). The reaction mixture was added to aq. 50% NaOH (2.45 mL). After 30 mins the mixture was added to aq. 50% NaOH (0.35 mL) dropwise. The reaction mixture was then heated to 30° C. for 3 hours. After cooling to room temperature, a saturated ammonium chloride (NH₄Cl) solution was added and the organic phase was separated. The aqueous phase was extracted with hexane. The combined organic phases were washed with NH₄Cl, then brine and dried (MgSO₄). Filtration and evaporation under reduced pressure followed by flash chromatography on silica (hexane/EtOAc, 98:2) afforded tert-butyl-2-((4Z,7Z,10Z,13Z,16Z,19Z)-docosa-4,7,10,13,16,19-hexaen-1-yloxy)butanoate (552 mg) containing impurities of t-butyl-2-brombutyrate:

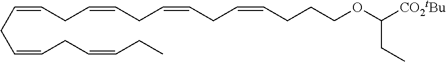 [SMILES: CC/C=C\C/C=C\C/C=C\C/C=C\C/C=C\C/C=C\CCCOC(CC)C(=O)OC(C)(C)C]

The tert-butylester (552 mg) was dissolved in formic acid (95%, 6.0 mL). The reaction mixture was left stirring at room temperature for 3 hours. The mixture was concentrated under vacuum and the residue purified by flash chromatography on silica gel (9:1 hexane/EtOAc containing 1% formic acid). Flash chromatography (hexane/EtOAc, 9:1, acidified with formic acid) eluted a mixture of product and the hydrolysed bromide. After evaporation the crude oil (291 mg) was dissolved in MTBE-ether (50 mL) and washed with sat. NaHCO₃ (3×25 mL), sat. NH₄Cl (25 mL) and brine (25 mL) and dried (MgSO₄). Filtration and evaporation gave the pure acid as a colourless oil (202 mg) in 19% yield.

δ_{H} (400 MHz) 0.9-1.0 (m, 6H), 1.6-1.9 (m, 4H), 2.0-2.1 (m, 2H), 2.1-2.2 (m, 2H), 2.7-2.9 (m, 10H), 2.35-3.45 (m, 1H), 3.55-3.65 (m, 1H), 3.75-3.85 (m, 1H) 5.2-5.54 (m, 12H)

δ_{C} (100 MHz) 9.5, 14.3, 20.5, 23.6, 25.4, 25.5, 25.8, 29.4, 70.1, 79.6, 126.9, 127.7, 127.96, 127.97, 128.0, 128.07, 128.13, 128.2, 128.4, 128.5, 129.2, 132.0, 177.5.

### EXAMPLE 18

### Preparation of (3Z,6Z,9Z,12Z)-pentadeca-(3,6,9,12)-tetraen-1-thiol

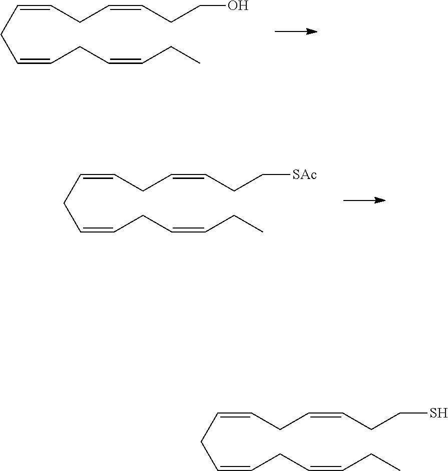

Diisopropyl azodicarboxylate (DIAD) (1.97 ml, 10.01 mmol) was added to a stirred solution of triphenylphosphine (2.75 g, 10.50 mmol) in THF (30 ml) at 0° C., and the mixture was stirred at this temperature for 30 mins. A solution of (3Z,6Z,9Z,12Z)-pentadeca-(3,6,9,12)-tetraen-1-ol (Flock et al., Acta chemical scandinavica, 1999, 53, 436) (1.8 g, 9.1 mmol) and thioacetic acid (715 μl, 10.01 mmol) in THF (10 ml) was added dropwise over 20 mins. The mixture was stirred for 1 hour at 0° C. and for an additional hour at ambient temperature. The mixture was concentrated and purified by flash chromatography on silica gel (95:5 hexane:EtOAc) to give thioacetic ester (1.2 g, 47%) as an oil.

The thioester (710 mg, 2.6 mmol) was dissolved in methanol (30 ml) and added to K₂CO₃ (1.06 g, 7.65 mmol). The mixture was stirred at room temperature for 2 hours before addition of 1M HCl, water and diethylether. The organic phase was separated and the aqueous phase extracted with diethylether (3×30 ml). The combined organic layers were washed with brine and dried (MgSO₄). Filtration and evaporation gave the thiol as an oil (520 mg, 85% yield).

### EXAMPLE 19

### Preparation of 2-((3Z,6Z,9Z,12Z)-pentadeca-3,6,9,12-tetraenylthio)butanoic acid [corresponding to Example 9 in U.S. Pat. No. 8,759,558] (BIZ 109)

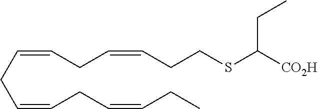 [SMILES: CC/C=C\C/C=C\C/C=C\C/C=C\CCSC(CC)C(=O)O]

An ice-cooled solution of (3Z,6Z,9Z,12Z)-pentadeca-(3,6,9,12)-tetraen-1-thiol as prepared in Example 18 (480 mg, 2.03 mmol) in dry dimethylformamide (DMF) (10 ml) was added to NaH (89 mg, 60% in mineral oil). The mixture was stirred at 0° C. for an additional 10 minutes before addition of ethyl bromobutyrate (330 μl, 2.3 mmol). The mixture was stirred at room temperature for 40 minutes, then the mixture was poured into a saturated NH₄Cl solution and extracted with hexane. The extract was washed with a saturated NH₄Cl solution, water and dried (MgSO₄). Filtration, evaporation under reduced pressure and purification by flash chromatography (hexane:EtOAC 98:2) afforded the ethyl ester (450 mg, 70%) as an oil:

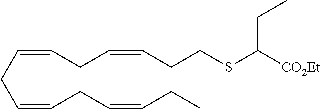 [SMILES: CC/C=C\C/C=C\C/C=C\C/C=C\CCSC(CC)C(=O)OCC]

The ester (270 mg, 0.85 mmol) was dissolved in ethanol (10 ml) and added to a solution of LiOH (267 mg, 6.4 mmol) in water (10 ml). The mixture was heated at 45° C. for 3 hours, cooled, added to water and 1M HCl until pH=2. The mixture was extracted with heptane (3×30 ml) and the extract was washed with brine, water and dried (MgSO₄). Filtration and evaporation followed by purification by filtration through a short plug of silica gel (hexane:EtOAc 9:1) and concentration under reduced pressure afforded the acid (80 mg).

δ_{H} (400 MHz) 0.95 (t, J=7.5, 3H), 1.02 (t, J=7.4, 3H), 1.65-1.75 (m, 1H), 1.85-1.95 (m, 1H), 2.06 (quintett, J=7.3, 2H), 2.30-2.40 (m, 2H), 2.60-2.75 (m, 2H), 2.75-2.85 (m, 6H), 3.18 (t, J=7.5, 1H), 5.20-5.45 (m, 8H).

δ_{C} (100 MHz) 11.9, 14.3, 20.6, 24.48, 25.53, 25.6, 25.7, 27.1, 31.3, 48.2, 127.0, 127.5, 127.8, 127.9, 128.4, 128.6, 129.7, 132.0, 178.7

### EXAMPLE 20

### Preparation of 2-methyl-2-((3Z,6Z,9Z,12Z)-pentadeca-3,6,9,12-tetraenylthio)-propanoic acid (BIZ 113)

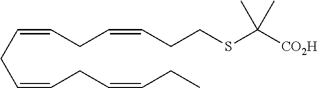 [SMILES: CC/C=C\C/C=C\C/C=C\C/C=C\CCSC(C)(C)C(=O)O]

An ice-cooled solution of (3Z,6Z,9Z,12Z)-pentadeca-(3,6,9,12)-tetraen-1-thiol (520 mg, 2.2 mmol) in dry dimethylformamide (DMF) (10 ml) was added to NaH (97 mg, 60% in mineral oil). The mixture was stirred at 0° C. for an additional 10 minutes before addition of 2-bromoisobutyrate (392 μl, 2.6 mmol). The mixture was stirred at room temperature for 40 minutes, then the mixture was poured into a saturated NH₄Cl solution and extracted with hexane. The extract was washed with a saturated NH₄Cl solution, water and dried (MgSO₄). Filtration, evaporation under reduced pressure and purification by flash chromatography (hexane:EtOAC 98:2) afforded the ester (270 mg) as an oil:

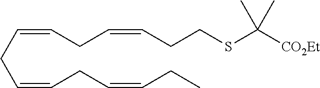 [SMILES: CC/C=C\C/C=C\C/C=C\C/C=C\CCSC(C)(C)C(=O)OCC]

The ester (270 mg) was dissolved in ethanol (10 ml) and added to a solution of LiOH (270 mg, 6.4 mmol) in water (10 ml). The mixture was heated at 65° C. for 4 hours, cooled, added to water and 1M HCl until pH=2. The mixture was extracted with hexane (3×30 ml) and the extract was washed with brine, water and dried (MgSO₄). Filtration and evaporation followed by purification by filtration through a short plug of silica gel (hexane:EtOAc 9:1) and concentration under reduced pressure afforded the acid (200 mg).

δ_{H} (400 MHz) 0.95 (t, J=7.5, 3H), 1.51 (s, 6H), 2.06 (quintet, J=7.3, 2H), 2.32 (quartet, J=7.0, 2H), 2.68 (t, J=7.3, 2H), 2.75-2.85 (m, 6H), 5.20-5-45 (m, 8H)

δ_{C} (100 MHz) 14.3, 20.5, 25.4, 25.5, 25.6, 25.7, 26.9, 27.7, 46.6, 127.0, 127.6, 127.8, 127.8, 127.9, 128.4, 128.6, 129.6, 132.0, 180.3

### EXAMPLE 21

### 3-oxa-(6Z,9Z,12Z,15Z,18Z)-heneicosa-(6,9,12,15,18)-pentaenoic acid (BIZ 108)

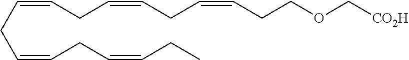 [SMILES: CC/C=C\C/C=C\C/C=C\C/C=C\C/C=C\CCOCC(=O)O]

3 -oxa-(6Z,9Z,12Z,15Z,18Z)-heneicosa-(6,9,12,15,18)-pentaenoic acid was prepared as described in the literature (see Flock et al., Acta Chemica Scandinavica, 1999, 53, 436 -Compound 21b)

δ_{H} (400 MHz) 0.95 (t, J=7.5, 3H), 2.05 (quintet, J=7.4, 2H), 2.40 (q, J=6.8, 2H), 2.7-2.9 (m, 8H), 3.56 (t, J=6.8, 2H), 4.14 (s, 2H), 5.20-5.60 (m, 10 H)

δ_{C} (100 MHz) 14.2, 20.5, 25.5, 25.6, 25.6, 25.7, 27.7, 67.7, 71.4, 125.2, 127.0, 127.8, 127.9, 128.0, 128.26, 128.31, 128.5, 130.4, 132.0, 175.0

### EXAMPLE 22

### Measuring the Effects of Compounds on the Activity of NF-κB Pathway using NF-κB Reporter Assay

Background: Corticosteroids like dexamethasone have for many years been used for treatment of a broad spectrum of inflammatory conditions of the eye due to their potent anti-inflammatory effect. However, steroids may cause severe side effects like cataract and raised intraocular pressure after prolonged use, which limits their therapeutic use in chronic diseases. Corticosteroids exert their anti-inflammatory effects through influencing multiple signal transduction pathways. Inhibition of the NF-κB pathway is central in their anti-inflammatory effect.

NF-κB (Nuclear Factor-Kappa B, NF-κB) is a heterodimeric protein composed of different combinations of members of the Rel family of transcription factors. The NF-κB/Rel family of transcription factors (p50, p65, c-Rel, etc.) are involved in stress, immune, and inflammatory responses. In unstimulated cells, the NF-κB dimers are sequestered in the cytoplasm by inhibitory IkB proteins. Proinflammatory cytokines such as TNF-α, LPS, growth factors, and antigen receptors activate I B kinase (IKK), which phosphorylates the IkB proteins. Phosphorylation of IkB leads to its degradation, freeing NF-κB complexes to translocate to the nucleus, bind to NF-κB DNA response elements, and induce the transcription of the target genes.

Description of assay: The NF-κB reporter (luc)-HEK293 cell line is designed for monitoring the nuclear factor Kappa B (NF-κB) signal transduction pathways. It contains a firefly luciferase gene driven by four copies of NF-κB response element located upstream of the minimal TATA promoter. After activation by pro-inflammatory cytokines or stimulants of lymphokine receptors, endogenous NF-κB transcription factors bind to the DNA response elements, inducing transcription of the luciferase reporter gene.

Cell Culture: NF-κB Reporter-HEK293 cells were cultured in MEM medium with 10% FBS, 1% non-essential amino acids, 1 mM Na-pyruvate, 1% Penn-strep, and 100 μg/ml of Hygromycin B.

Assay Conditions: To perform the NF-κB luciferase reporter assay, NF-κB Reporter-HEK293 cells were seeded at 40,000 cells per well into white clear-bottom 96-well microplate in 45 μl of growth medium without Hygromycin B. Cells were incubated at 37° C. and 5% CO₂ overnight to allow them to recover and reattach. The following day a series of dilutions of compounds were made in assay medium (MEM medium with 0.5% FBS, 1% non-essential amino acids, 1 mM Na-pyruvate, 1% Penn-strep). The medium was removed from the wells and 45 μl of diluted compound was added to the cells. Assay medium with DMSO was added to the untreated control wells and cell-free control wells. The final concentration of DMSO was 0.25%. The cells were incubated overnight at 37° C. in CO2 incubator. The next day 5 μl of assay medium with TNFα was added to the wells. The final concentration of TNFα was 5 ng/ml. Cells were treated for 5 hours at 37° C. in a CO2 incubator.

After treatment, cells were then lysed and a luciferase assay was performed using the ONE-Step luciferase assay system. In brief, 50 μl of One-Step Luciferase reagent was added per well and the plate rocked at room temperature for 30 minutes. Luminescence was measured using a luminometer (BioTek Synergy™ 2 microplate reader).

Data Analysis: Reporter assays were performed in triplicate at each concentration. The luminescence intensity data were analyzed using the computer software, Graphpad Prism. In the absence of the compound, the luminescence intensity (Lt) in each data set was defined as 100%. In the absence of cells, the luminescence intensity (L_{b}) in each data set was defined as 0%. The percent luminescence in the presence of each compound was calculated according to the following equation: % Luminescence=(L−L_{b})/(Lₜ−L_{b}), where L=the luminescence intensity in the presence of the compound, L_{b}=the luminescence intensity in the absence of cells, and Lₜ=the luminescence intensity in the absence of the compound. The values of % luminescence versus a series of compound concentrations were then plotted using non-linear regression analysis of a Sigmoidal dose-response curve generated with the equation Y=B+(T−B)/1+10^{((LogEC50-X)×Hill Slope)}, where Y=percent luminescence, B=minimum percent luminescence, T=maximum percent luminescence, X=logarithm of compound and Hill Slope=slope factor or Hill coefficient. The IC50 value was determined by the concentration causing a half-maximal percent activity.

Results: The results from the NF-κB assay clearly show that the tested compounds at 10 μM concentration have a surprisingly potent inhibitory effect of the TNF-α activated NF-κB pathway as shown in FIG. 4. The compounds are more potent inhibitors of the NF-κB pathway than dexamethasone.

The inhibitory effect of the tested compounds BIZ-110 and BIZ-101 on the Nf-κB pathway is dose dependent as seen in FIG. 5. In fact the Nf-κB pathway can be completely inhibited at high concentration using these two compounds which is not the case with dexamethasone.

### EXAMPLE 23

### Screening of PPAR Activity

To screen the human PPARα, PPARδ and PPARγ ligand activity of the compounds a stable reporter cell line was used (HeLa cell line). The stable reporter cell line express respectively a chimeric protein containing the ligand binding domain (LBD) of human PPARα, human PPARδ and human PPARγ fused to the yeast transactivator GAL4 DNA binding domain (DBD). The luciferase (Luc) reporter gene is driven by a pentamer of the GAL4 recognition sequence in front of a β-globin promoter. The use of GAL4-PPARα, GAL4-PPARδ and GAL4-PPARγ chimeric receptors allows for elimination of background activity from endogenous receptors and quantification of relative activity across the three PPAR subtypes with the same reporter gene. The PPAR selectivity of the samples is determined by comparison to known drug references (GW7647) for PPARα, L-165041 for PPARδ and BRL49653 for PPARγ and a negative control (0.1% DMSO). Luciferase activity was measured by a luminometer and luciferase activity was expressed as relative light units (RLU).

Day 1: Seed 96—well plate with PPAR cells. Cell appearance was checked using optical microscopy. Cells were cultivated at confluency (80-100%).

Day 2: The test articles dissolved in DMSO were added to the cells. The controls (positive controls and the solvent control) were included in each individual plate. The cells were incubated for an additional 24 hrs after addition of test articles before analysis.

Day 3: To determine the PPAR subtype activity of the tested compounds, the percentage of PPAR ligand activity was calculated for each tested compound as follows:

Percentage of PPARα activity for the tested compound in 5 μM concentration=(RLUX_{comp}×100)/RLU_{Gw7647} where RLU_{GW7647} is the luminescence measured from PPARα cells incubated with 1 μM GW7647 and expressed as Relative Light Units. The activity of 1 μM GW7647 is set to 100%

Percentage of PPARγ activity for the tested compound in 5 μM concentration=(RLUX_{comp}×100)/RLU_{L165041} where RLU_{L165041} is the luminescence measured from PPARδ cells incubated with 1 μM L and expressed as Relative Light Units. The activity of 1 μM L165041 is set to 100%

Percentage of PPARγ activity for the tested compound in 5 μM concentration=(RLUX_{comp}×100)/RLU_{BRL49653} where RLU_{BRL49653} is the luminescence measured from PPARγ cells incubated with 1 μM L and expressed as Relative Light Units. The activity of 1 μM BRL49653 is set to 100%

Results: The compounds tested in this assay were: BIZ-101, BIZ-102, BIZ-108, BIZ-109, BIZ-111, BIZ-112 and BIZ-113. The results showed that the tested compounds had no activity on PPARδ (results are not included). However, all compounds except BIZ-108 had potent activity on PPARα (FIG. 6) with response at the same level as GW7647. The results also show that most of the compounds tested activated PPARγ except for the unsubstituted derivative BIZ-108 (FIG. 7). The PPARγ activation is more potent in this stable reporter HeLA cell line than in the transient transfected HEK293 cell line described in Examples 6 and 7. Several differences in the assays can help to explain these results. BIZ-108 is unsubstituted in the α-position and was only added for comparison purposes. The results clearly show that a potent PPARα and PPARγ activation requires one or two substituents in the α-position of fatty acid derivatives.

### EXAMPLE 24

### Assay for Measuring Effects of Compound BIZ-101 During Conditions of Oxidative Stress in a Retinal Pigment Epithelial Cell Line (ARPE-19)

In order to evaluate the potential of BIZ-101 to protective the eye from oxidative stress damage the compound was tested in a cell based assay. Oxidative stress was induced in an ARPE-19 cell line using tert-butyl hydroperoxide at different concentrations. The cellular viability was kinetically monitored and measured by analysing the plasma membrane integrity based on the incorporation of a non-permeant and fluorescent DNA intercalating agent that selectively stain cytolytic cells with comprised plasma membranes.

Procedure: The ARPE-19 cell line was seeded and cultured in DMEM-F12 medium+10% SVF for 24 hrs in a 96 well plate. Cells were pre-treated or not with either fenofibric acid (25 μM) or compound BIZ-101 (10 μM) during this 24 hr period. After 24 hrs, the different concentrations of t-butyl hydroperoxide were added in the presence of a fluorescent DNA intercalating agent for the following monitoring. Live content time-lapse imaging was performed with a sampling rate of 1 image every 2 hr over a 96 hrs period. The number of fluorescent/cytolytic cells were counted and reported during the experiment. The treatment conditions were tested in one experiment session in a triplicate format.

Handling and solubilisation of compounds: Fenofibric acid was used at 25 μM and was ordered from Sigma Aldrich. The day of the pre-treatment, a 25 mM stock solution of fenofibric acid was prepared in DMSO and diluted in the complete culture medium at 25 μM. 100 μL of this solution or 100 μL of complete culture medium+0.1% DMSO replaced the 100 μL of the complete culture medium already present in the well. The day of the treatment with t-butyl hydroperoxide, a 2 times concentrated solution of fenofibric acid was freshly diluted in the complete culture medium and 50 μL of this solution or 50 μL of complete culture medium+0.2% DMSO were added to the 100 μL of the culture medium already present in the well.

Compound BIZ-101 was tested at a final concentration of 10 _{μ}M. A 10 mM predilution was prepared in DMSO from a 10 mM stock solution. The 10 mM solution of BIZ 101 was diluted in the complete culture medium at 10 μM. 100 μL of this solution or 100 μL of the complete culture medium+0.1% DMSO replaced the 100 μL of the complete culture medium already present in the well. The day of the treatment with t-butyl hydroperoxide, a 2 times concentrated solution of BIZ 101 was freshly diluted in the complete culture medium and 50 μL of this solution or 50 μL of complete culture medium+0.2% DMSO were added to the 100 μL of the culture medium already present in the well.

Preparation of the tert-butyl hydroperoxide [tBHP] solution: 0, 0.01 mM, 0.03 mM, 0.1 mM, 0.3 mM, 1 mM, 3 mM and 10mM. A 4 times concentrated solution of tBHP was freshly diluted in the complete culture medium for each of the concentrations to be tested. 50 μL of the 4 times concentrated solution was added to the 150 μL of the cultured medium.

Results: The addition of tBHP induced a rapid, severe and dose dependent cytotoxic effect on the ARPE-19 cells as shown in FIG. 8A. A dose-dependent effect could be observed and 24 hrs after addition of tBHP the complete cytolysis of ARPE-19 cells was induced even with the lowest concentration of tBHP (FIGS. 8A and 8D).

Pre-treatment with either fenofibric acid or BIZ-101 slightly decreased the dose dependent cytotoxicity of tBHP 6 hrs after addition of tBHP. The protective effects of fenofibric acid and BIZ-101 were more efficient 24 hrs after addition of t-BHP, and remained stable until the end of the monitoring time (FIG. 8B, 8C, 8D).

The study clearly shows that BIZ-101 has a protective effect on retinal pigment epithelial cells (ARPE-19 cells) during conditions of oxidative stress. The effect is much more potent than the effect seen for fenofibric acid which is known to have therapeutic effects in treatment of diabetic retinopathy.

### EXAMPLE 25

### Assay for Measuring Effects of Compound BIZ-102 During Conditions of Oxidative Stress in a Retinal Pigment Epithelial Cell Line (ARPE-19)

In order to evaluate the potential of BIZ-102 to protect the eye from oxidative stress damage the compound was tested in a cell based assay similar to Example 24 with the following changes. The oxidative stress in the ARPE-19 cell line was induced by using 3 different concentrations of tBHP: 10 μM, 30 μM and 100 μM and BIZ-102 was tested in 2 different concentrations: 1 μM and 10 μM.

Procedure: The ARPE-19 cell line was seeded and cultured in DMEM-F12 medium+10% SVF for 24 hrs in a 96 well plate. Cells were pre-treated or not with compound BIZ-102 (1 μM and 10 μM) during this 24 hr period. After 24 hrs, the different concentrations of t-butyl hydroperoxide were added in the presence of a fluorescent DNA intercalating agent for the following monitoring. Live content time-lapse imaging was performed with a sampling rate of 1 image every 2 hr over a 96 hrs period. The number of fluorescent/cytolytic cells were counted and reported during the experiment. The treatment conditions were tested in one experiment session in a triplicate format.

Compound BIZ-102 was tested at a final concentration of 1 and 10 μM. A 10 mM pre-dilution was prepared in DMSO from a 20 mM stock solution. The day of the pre-treatment with BIZ-102 and the day of the treatment with t-BHP, a 1000 times concentrated solution of BIZ-102 for each concentration to be tested was prepared in DMSO. The day of the pre-treatment, a one time concentrated solution for each concentration to be tested was prepared by diluting the 1000 times concentrated solution in complete culture medium. 100 μL of those solutions replaced the 100 μL of the culture medium already present in the well. The day of the treatment with t-BHP, for each concentration to be tested, a 2 times concentrated solution was freshly diluted in the complete culture medium and 50 μL of this solution was added on the 100 μL of the complete culture medium already present in the well.

Results: The results show that BIZ-102 at the highest concentration (10 μM) exhibited a significant inhibition of the cytotoxicity induced by both 10 μM and 30 μM of t-BHP but not for the highest concentration of t-BHP (FIG. 9A, 9B, 9C). The protective effect remained stable until the end of the monitoring time. In FIG. 9D the effects of BIZ-102 12 hrs after addition of tBHP is shown. The study clearly shows that BIZ-102 has a protective effect on retinal pigment epithelial cells (ARPE-19 cells) during conditions of oxidative stress.

1. A lipid compound of formula (I), or a pharmaceutically acceptable salt thereof, for the prevention and/or treatment of an ophthalmic disorder: 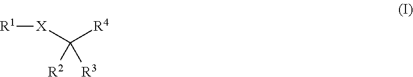
wherein
R¹ is either a C₉ to C₂₂ alkyl group, or a C₉ to C₂₂ alkenyl group having from 1 to 6 double bonds;
R² is selected from the group consisting of a halogen atom, a hydroxy group, an alkyl group, an alkoxy group, an alkylthio group, a carboxy group, an acyl group, an amino group, and an alkylamino group;
R³ is a hydrogen atom, or a group R²;
R⁴ is a carboxylic acid or a derivative thereof; and
X is methylene (—CH₂—), or an oxygen or sulfur atom.

2. A lipid compound for use as claimed in claim 1, wherein said ophthalmic disorder is selected from: cataracts, corneal neovascularization, dry eye syndrome, glaucoma, keratocornus, dry eye disease, and Sjøgren's syndrome.

3. A lipid compound for use as claimed in claim 1, wherein said ophthalmic disorder is an ocular inflammatory disease (MD).

4. A lipid compound for use as claimed in claim 3, wherein said ocular inflammatory disease is allergic conjunctivitis, hayfever, uveitis, scleritis, episcleritis, endophthalmitis, optic neuritis, keratitis, orbital pseudotumor, retinal vasculitis, or chronic conjunctivitis.

5. A lipid compound for use as claimed in claim 4, wherein uveitis is anterior uveitis, intermediate uveitis, posterior uveitis or panuveitis uveitis.

6. A lipid compound for use as claimed in claim 1, wherein said ophthalmic disorder is an inflammatory, autoimmune, vascular and/or infectious disease of the posterior segment of the eye.

7. A lipid compound for use as claimed in claim 1, wherein said ophthalmic disorder is a retinal degenerative disease.

8. A lipid compound for use as claimed in claim 7, wherein said disorder is age-related macular degeneration (including dry-form AMD and wet-form AMD), diabetic retinopathy, diabetic macular edema, retinitis pigmentosa, Stargardt's disease, Usher syndrome, or Bardet-Biedl syndrome.

9. A lipid compound for use as claimed in claim 1, wherein said ophthalmic disorder is diabetic retinopathy or age-related macular degeneration (AMD).

10. A lipid compound for use as claimed in any one of the preceding claims, wherein in formula (I), X is —CH₂—.

11. A lipid compound for use as claimed in any one of claims 1 to 9, wherein in formula (I), X is an oxygen or sulphur atom.

12. A lipid compound for use as claimed in any one of the preceding claims, wherein in formula (I), R² and/or R³ is an alkyl group, preferably a straight-chained or branched C₁₋₆ alkyl (e.g. C₁₋₃ alkyl), e.g. methyl or ethyl.

13. A lipid compound for use as claimed in any one of the preceding claims, wherein in formula (I), R³ is a hydrogen atom.

14. A lipid compound for use as claimed in any one of the preceding claims, wherein in formula (I), R⁴ is a derivative of a carboxylic acid selected from a carboxylic ester, a carboxylic anhydride, a carboxamide, a monoglyceride, a diglyceride, a triglyceride, and a phospholipid, preferably wherein R⁴ is a derivative of a carboxylic acid which is a carboxylic ester.

15. A lipid compound for use as claimed in any one of the preceding claims, wherein R¹ is a C₁₀ to C₂₂ alkyl group, preferably a C₁₂ to C₂₀ alkyl group, more preferably a C₁₂ to C₁₆ alkyl group, e.g. a C₁₄ alkyl group.

16. A lipid compound for use as claimed in claim 15, wherein X is an oxygen or sulphur atom.

17. A lipid compound for use as claimed in any one of claims 1 to 14, wherein in formula (I), R¹ is a C₉ to C₂₂ alkenyl group having from 1 to 6 double bonds, preferably a straight-chained C₉ to C₂₂ alkenyl group.

18. A lipid compound for use as claimed in claim 17, wherein R¹ is a C₁₀ to C₂₂ alkenyl group, more preferably a C₁₂ to C₁₉ alkenyl group, e.g. a C₁₉ alkenyl group, more preferably a C₁₄ to C₁₈ alkenyl group.

19. A lipid compound for use as claimed in claim 18, wherein R¹ is a C₁₅ or C₁₇ alkenyl group.

20. A lipid compound for use as claimed in any one of claims 17 to 19, wherein R¹ is a straight-chained ω-3 C₉ to C₂₂ alkenyl group having from 1 to 6 double bonds.

21. A lipid compound for use as claimed in any one of claims 17 to 19, wherein R¹ is a straight-chained ω-6 C₉ to C₂₂ alkenyl group having from 1 to 5 double bonds.

22. A lipid compound for use as claimed in any one of claims 17 to 21, wherein at least two double bonds are present in group R¹, preferably wherein at least one pair of successive double bonds is interrupted by no more than one methylene group, e.g. wherein each pair of consecutive double bonds is interrupted by no more than one methylene group.

23. A lipid compound for use as claimed in any one of claims 17 to 22, wherein all double bonds present in group R¹ are either in the E- or Z-configuration, preferably wherein all double bonds are in the Z-configuration.

24. A lipid compound for use as claimed in any one of claims 17 to 23, wherein in formula (I), X is —CH₂— and R¹ is a C₉ to C₂₂ alkenyl having 2 to 6 double bonds, e.g. a C₁₉, C₁₇, or C₁₅ alkenyl having 6, 5, 4 or 3 double bonds.

25. A lipid compound for use as claimed in any one of claims 17 to 23, wherein in formula (I), X is S or O and R¹ is a C₉ to C₂₂ alkenyl having 2 to 6 double bonds, e.g. a C₂₂, C₂₀, C₁₈, or C₁₅ alkenyl having 6, 5, 4 or 3 double bonds.

26. A lipid compound for use as claimed in any one of claims 15 to 25, wherein said compound is derived from an ω-3 PUFA or an ω-6 PUFA, e.g. derived from omega-3 docosahexaenoic acid (omega-3 DHA), omega-3 eicosapentaenoic acid (omega-3 EPA), omega-3 docosapentaenoic acid (omega-3 DPA), omega-3 α-linoleic acid (omega-3 ALA), omega-6 arachidonic acid (omega-6 AA), omega-6 docosapentaenoic acid (omega-6 DPA), omega-6 dihomo-y-linolenic acid (omega-6 DGLA), or omega-6 linoleic acid (omega-6 LA).

27. A lipid compound for use as claimed in any one of claims 1 to 9, wherein said compound is selected from any of the following, or their pharmaceutically acceptable salts: 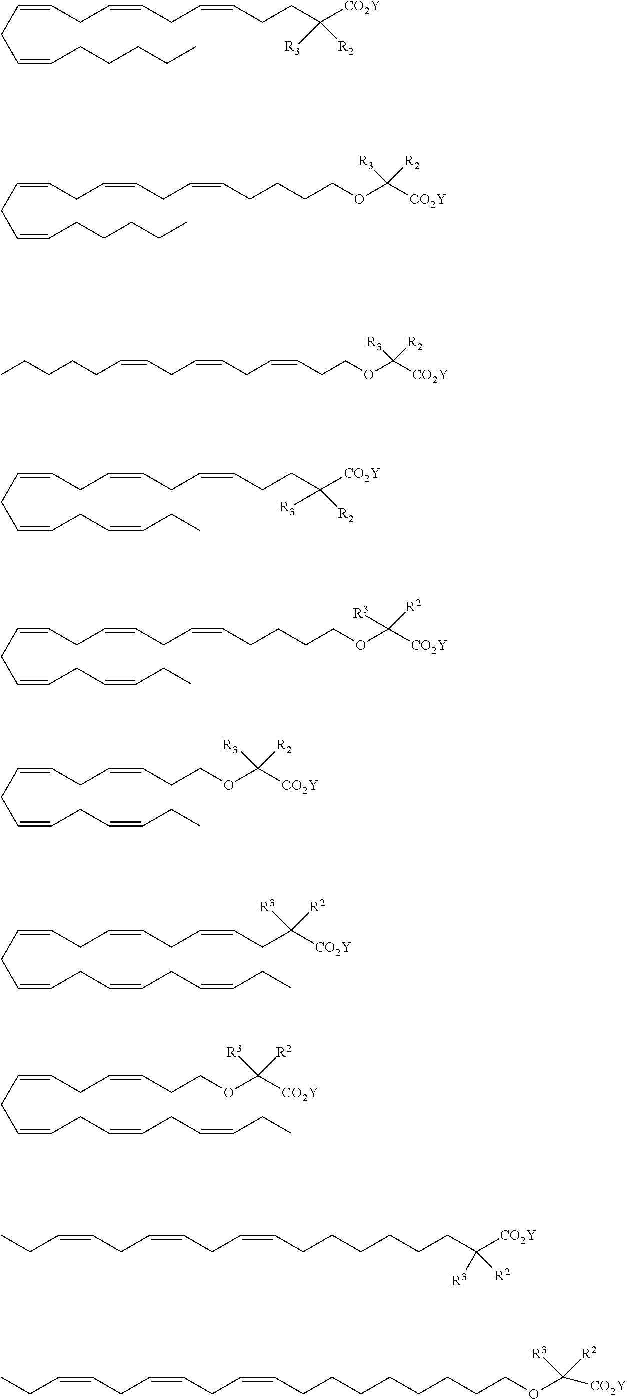
in which R² and R³ are as defined in any one of claims 1, 12 and 13; and
Y is either hydrogen or an alkyl group, e.g. C₁₋₆ alkyl.

28. A lipid compound for use as claimed in any one of claims 1 to 9, wherein said compound is: 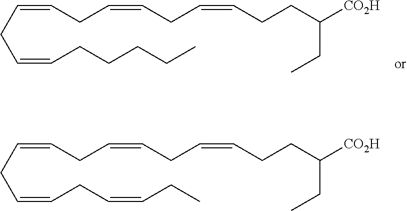 [SMILES: CCCCC/C=C\C/C=C\C/C=C\C/C=C\CCC(CC)C(=O)O ; CC/C=C\C/C=C\C/C=C\C/C=C\C/C=C\CCC(CC)C(=O)O]
or a pharmaceutically acceptable salt, or ester (e.g. C₁₋₆ alkyl ester) thereof.

29. A lipid compound for use as claimed in any one of claims 1 to 9, wherein said compound is: 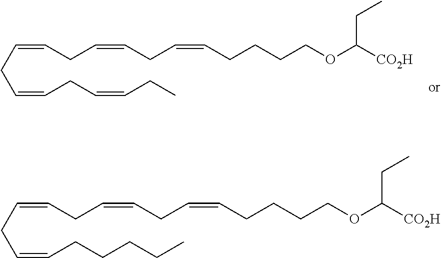 [SMILES: CC/C=C\C/C=C\C/C=C\C/C=C\C/C=C\CCCCOC(CC)C(=O)O ; CCCCC/C=C\C/C=C\C/C=C\C/C=C\CCCCOC(CC)C(=O)O]
or a pharmaceutically acceptable salt, or ester (e.g. C₁₋₆ alkyl ester) thereof.

30. Use of a lipid compound of formula (I), or a pharmaceutically acceptable salt thereof, as defined in any one of claims 1 and 10 to 29 in the manufacture of a medicament for use in the prevention and/or treatment of an ophthalmic disorder as defined in any one or claims 1 to 9.

31. An ophthalmic composition for topical administration to at least one surface of the eye, said composition comprising a lipid compound of formula (I), or a pharmaceutically acceptable salt thereof, as defined in any one of claims 1 and 10 to 29 together with at least one pharmaceutically acceptable carrier or excipient adapted for topical administration.

32. An ophthalmic composition as claimed in claim 31 which is provided in the form of eye drops or a gel.

33. An ophthalmic composition as claimed in claim 31 or claim 32 which is provided in the form of an emulsion.

34. A method of preventing and/or treating an ophthalmic disorder as defined in any one of claims 1 to 9, said method comprising the step of administering to a patient in need thereof (e.g. a human subject) a pharmaceutically effective amount of a lipid compound of formula (I), or a pharmaceutically acceptable salt thereof, as defined in any one of claims 1 and 10 to 29.

35. An omega-6 lipid compound of formula (II), or a pharmaceutically acceptable salt thereof: 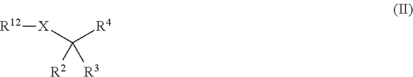
wherein
R¹² is a C₉ to C₂₂ alkenyl group having from 1 to 5 double bonds (e.g. 2 to 5 double bonds) in which:
the first double bond counting from the ω-end is at carbon 6; and
where two or more double bonds are present, at least one pair of consecutive double bonds is interrupted by a single methylene group;
R² is selected from the group consisting of a halogen atom, a hydroxy group, an alkyl group, an alkoxy group, an alkylthio group, a carboxy group, an acyl group, an amino group, and an alkylamino group, preferably R² is an alkyl group;
R³ is a hydrogen atom, or a group R²;
R⁴ is a carboxylic acid or a derivative thereof selected from a carboxylic ester, a carboxylic anhydride, a carboxamide, a monoglyceride, a diglyceride, a triglyceride, and a phospholipid; and
X is methylene (—CH₂—), or an oxygen or sulfur atom.

36. An omega-6 lipid compound as claimed in claim 35, wherein in formula (II), X is —CH₂—.

37. An omega-6 lipid compound as claimed in claim 35, wherein in formula (II), X is an oxygen or sulphur atom.

38. An omega-6 lipid compound as claimed in any one of claims 35 to 37, wherein in formula (II), R² and/or R³ is an alkyl group, preferably an unsubstituted, straight-chained or branched C₁₋₆ alkyl (e.g. C₁₋₃ alkyl), e.g. methyl or ethyl.

39. An omega-6 lipid compound as claimed in any one of claims 35 to 38, wherein in formula (II), R³ is a hydrogen atom.

40. An omega-6 lipid compound as claimed in any one of claims 35 to 39, wherein in formula (II), R⁴ is a derivative of a carboxylic acid which is a carboxylic ester.

41. An omega-6 lipid compound as claimed in any one of claims 35 to 40, wherein in formula (II), R¹ is a straight-chained C₉ to C₂₂ alkenyl group.

42. An omega-6 lipid compound as claimed in claim 41, wherein R¹ is a C₁₀ to C₂₂ alkenyl group, more preferably a C₁₂ to C₁₉ alkenyl group, e.g. a C₁₉ alkenyl group, more preferably a C₁₄ to C₁₈ alkenyl group.

43. An omega-6 lipid compound as claimed in claim 42, wherein R¹ is a C₁₅ or C₁₇ alkenyl group.

44. An omega-6 lipid compound as claimed in any one of claims 35 to 43, wherein at least two double bonds are present in group R¹, preferably wherein at least one pair of successive double bonds is interrupted by no more than one methylene group, e.g. wherein each pair of consecutive double bonds is interrupted by no more than one methylene group.

45. An omega-6 lipid compound as claimed in any one of claims 35 to 44, wherein all double bonds present in group R¹ are either in the E- or Z-configuration, e.g. wherein all double bonds are in the Z-configuration.

46. An omega-6 lipid compound as claimed in any one of claims 35 to 45, wherein said compound is derived from an ω-6 PUFA, e.g. derived from (all-Z)-5,8,11,14-icosatetraenoic acid, (all-Z)-4,7,10,13,16-docosapentaenoic acid, (all-Z)-8,11,14-eicosatrienoic acid, or (all-Z)-9,12-octadecadienoic acid.

47. An omega-6 lipid compound as claimed in claim 35 which is selected from any of the following, or their pharmaceutically acceptable salts: 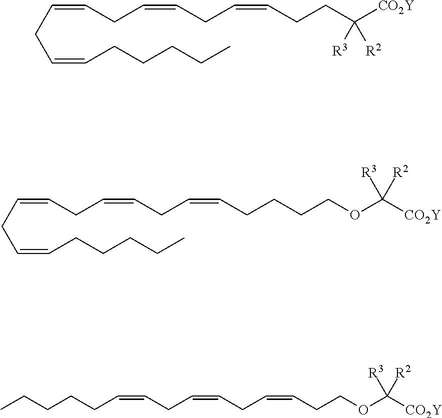
in which R² and R³ are as defined in any one of claims 35, 38 and 39; and
Y is either hydrogen or an alkyl group, e.g. C₁₋₆ alkyl.

48. An omega-6 lipid compound as claimed in claim 35 which is selected from the following compounds and their pharmaceutically acceptable salts:

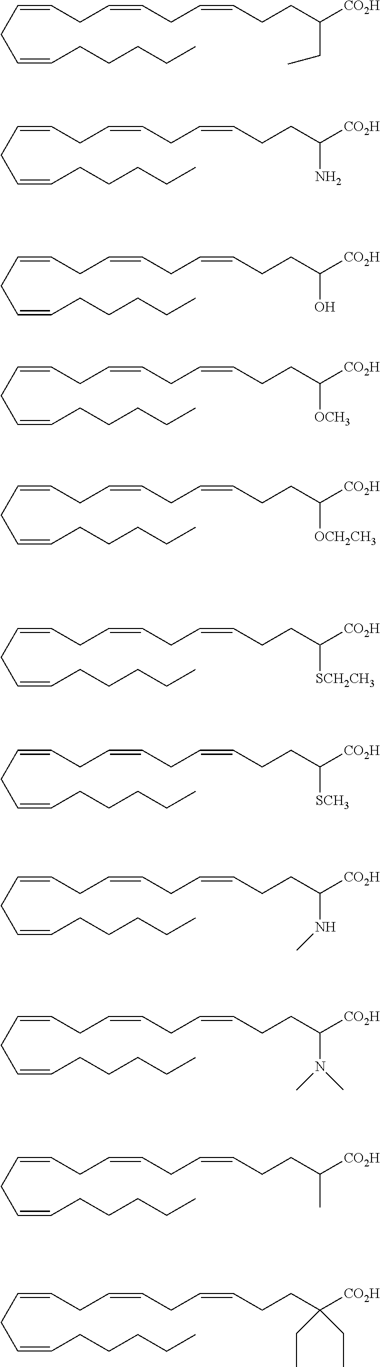

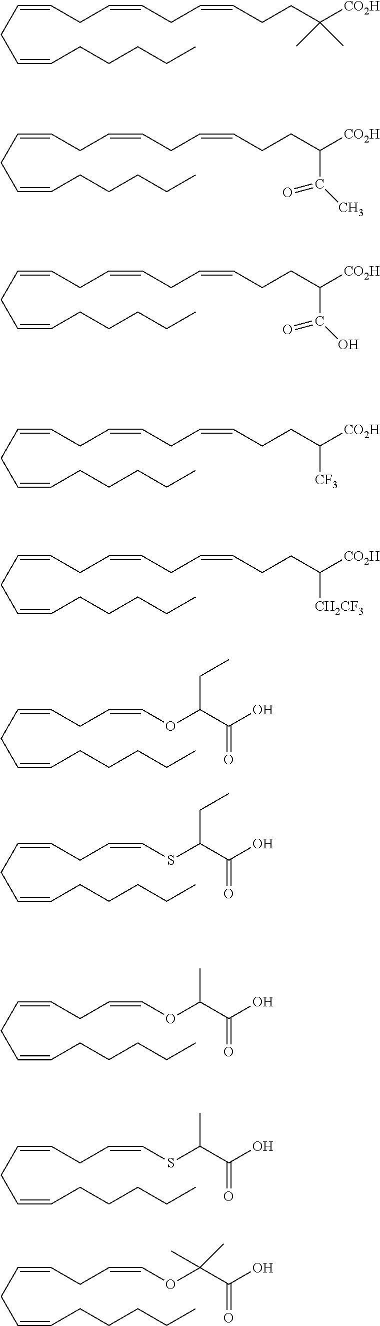

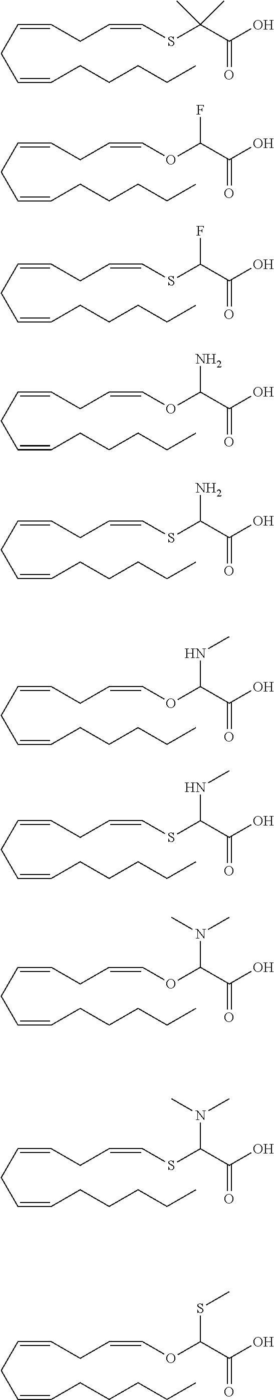

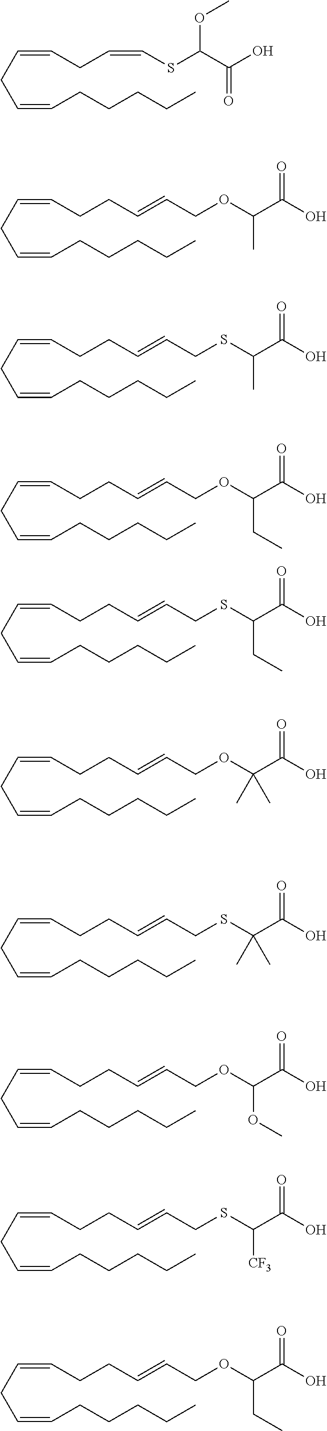

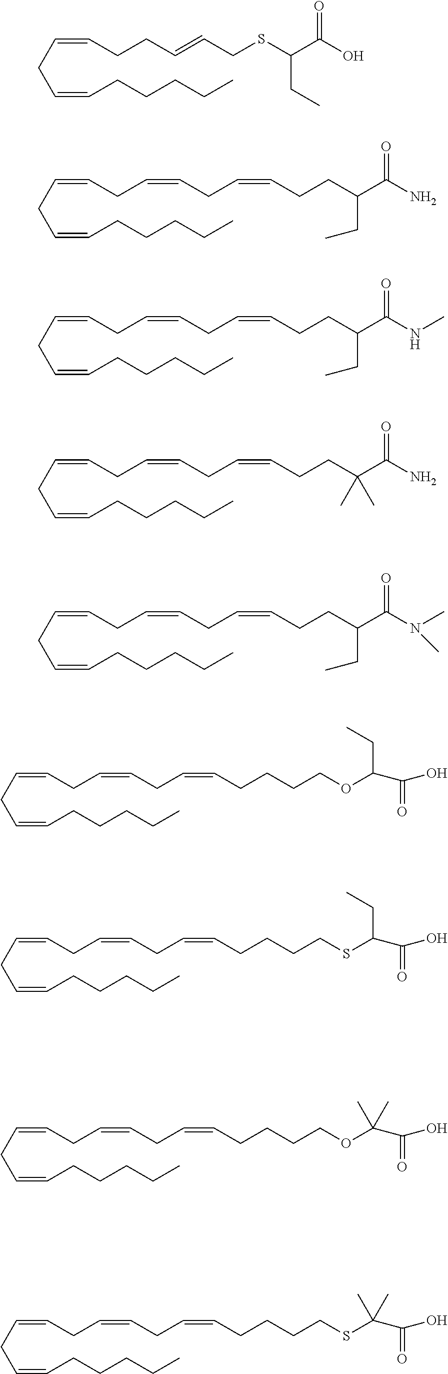

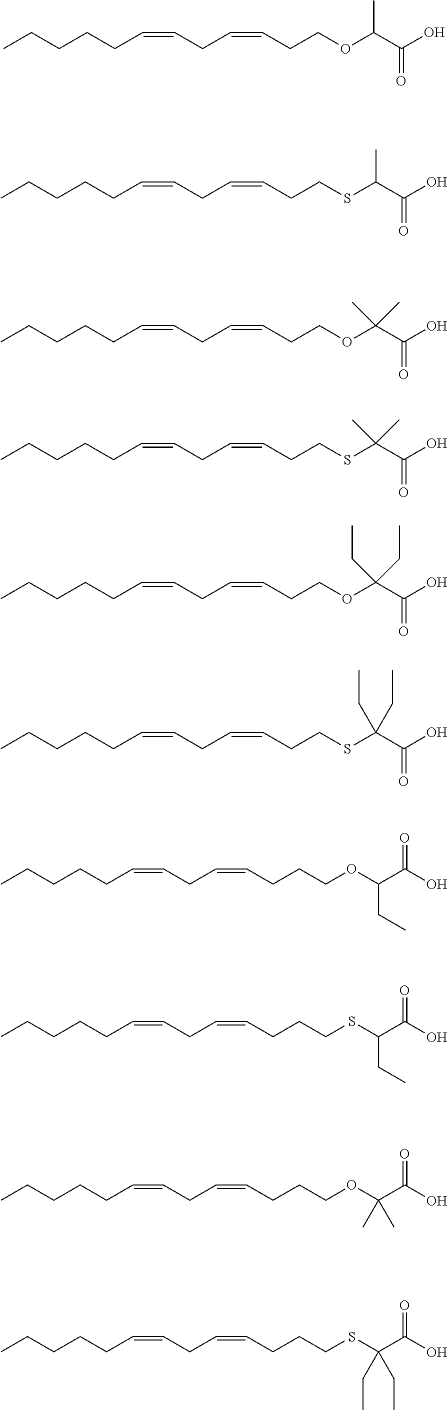

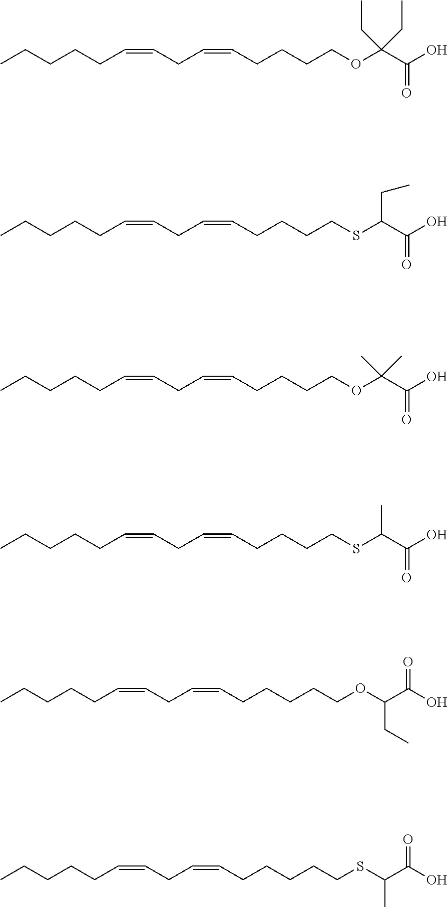 [SMILES: CCCCC/C=C\C/C=C\CCCCOC(CC)(CC)C(=O)O ; CCCCC/C=C\C/C=C\CCCCSC(CC)C(=O)O ; CCCCC/C=C\C/C=C\CCCCOC(C)(C)C(=O)O ; CCCCC/C=C\C/C=C\CCCCSC(C)C(=O)O ; CCCCC/C=C\C/C=C\CCCCCOC(CC)C(=O)O ; CCCCC/C=C\C/C=C\CCCCCSC(C)C(=O)O]

49. An omega-6 lipid compound as claimed in claim 35 which is 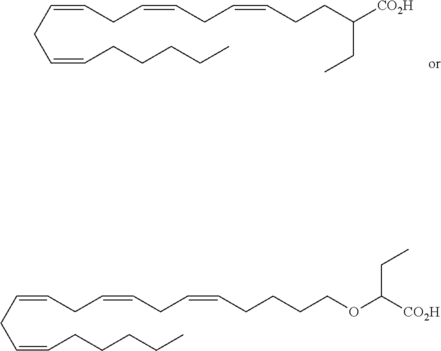 [SMILES: CCCCC/C=C\C/C=C\C/C=C\C/C=C\CCC(CC)C(=O)O ; CCCCC/C=C\C/C=C\C/C=C\C/C=C\CCCCOC(CC)C(=O)O]
or a pharmaceutically acceptable salt, or ester (e.g. a C₁₋₆ alkyl ester) thereof.

50. An omega-6 lipid compound as claimed in any one of claims 35 to 49 for use as a medicament.

51. A pharmaceutical composition comprising an omega-6 lipid compound as claimed in any one of claims 35 to 49, together with one or more pharmaceutically acceptable carriers, excipients or diluents.

52. An omega-6 lipid compound as claimed in any one of claims 35 to 49 for use in the prevention and/or treatment of an ophthalmic disorder.

53. An omega-6 lipid compound for use as claimed in claim 52, wherein said ophthalmic disorder is as defined in any one of claims 2 to 9.

54. A lipid composition comprising an omega-6 lipid compound of formula (II), or a pharmaceutically acceptable salt thereof, as claimed in any one of claims 35 to 49.

55. A lipid composition as claimed in claim 54 for use as a medicament, e.g. for use in the prevention and/or treatment of an ophthalmic disorder.

56. A lipid composition for use as claimed in claim 55, wherein said ophthalmic disorder is as defined in any one of claims 2 to 9.

57. Use of an omega-6 lipid compound of formula (II), or a pharmaceutically acceptable salt thereof, as claimed in any one of claims 35 to 49 in the manufacture of a medicament for use in the prevention and/or treatment of an ophthalmic disorder, in particular a retinal degenerative disorder or ocular inflammatory disease.

58. A method of preventing and/or treating an ophthalmic disorder, in particular a retinal degenerative disorder or ocular inflammatory disease, said method comprising the step of administering to a patient in need thereof (e.g. a human subject) a pharmaceutically effective amount of an omega-6 lipid compound of formula (II), or a pharmaceutically acceptable salt thereof, as claimed in any one of claims 35 to 49.

59. An oil-in-water emulsion, e.g. an oil-in-water microemulsion, comprising an omega-6 lipid compound of formula (II), or a pharmaceutically acceptable salt thereof, as claimed in any one of claims 35 to 49.
